(19) United States
(12) Patent Application Publication
(10) Pub. No.: US 2013/0303826 A1
(43) Pub. Date: Nov. 14, 2013

(54) PROGNOSTIC SIGNATURE FOR ORAL SQUAMOUS CELL CARCINOMA

(71) Applicants: Igor Jurisica, Toronto (CA); Suzanne Kamel-Reid, Toronto (CA); David Levi Waldron, Jamaica Plain, MA (US); Patricia Reis, Botucatu (BR)

(72) Inventors: Igor Jurisica, Toronto (CA); Suzanne Kamel-Reid, Toronto (CA); David Levi Waldron, Jamaica Plain, MA (US); Patricia Reis, Botucatu (BR)

(73) Assignee: University Health Network, Toronto (CA)

(21) Appl. No.: 13/979,072

(22) PCT Filed: Jan. 11, 2012
(86) PCT No.: PCT/CA2012/000030

§ 371 Date: Jul. 10, 2013

Related U.S. Application Data
(60) Provisional application No. 61/431,512, filed on Jan. 11, 2011.

(51) Int. Cl. C12Q 1/68; A61N 5/00; G01N 33/68

(52) U.S. Cl. CPC C12Q 1/6886; G01N 33/6893; A61N 5/00
USPC 600/1; 435/6.12; 506/9; 435/6.11; 435/7.1

(57) ABSTRACT

The present disclosure describes methods and compositions for diagnosing or predicting likelihood of a OSCC recurrence in a subject having undergone OSCC resection comprising: a) determining an expression level of one or more biomarkers selected from Table 4, 5 and/or 7, optionally MMP1, COL4A1, THBS2 and/or P4HA2 in a test sample from the subject, the one or more biomarkers comprising at least one of THBS2 and P4HA2, and b) comparing the expression level of the one or more biomarkers with a control, wherein a difference or a similarity in the expression level of the one or more biomarkers between the test sample and the control is used to diagnose or predict the likelihood of OSCC recurrence in the subject In particular, the present disclosure describes methods and compositions using a four-gene biomarker signature that can predict recurrence of oral squamous cell carcinoma in subjects that have histologically normal surgical resection margins.

### FIELD

The disclosure relates to methods, compositions and kits for diagnosing or predicting a likelihood of Oral Squamous Cell Carcinomas (OSCC) recurrence in a subject and specifically to biomarkers, the expression of which are useful for diagnosing or predicting a likelihood of OSCC recurrence.

### INTRODUCTION

Oral Squamous Cell Carcinoma (OSCC) is a major cause of cancer death worldwide, which is mainly due to disease recurrence leading to treatment failure and patient death.

OSCC accounts for 24% of all head and neck cancers (1). Currently available protocols for treatment of OSCCs include surgery, radiotherapy and chemotherapy. Complete surgical resection is the most important prognostic factor (2), since failure to completely remove a primary tumor is the main cause of patient death. Accuracy of the resection is based on the histological status of the margins, as determined by microscopic evaluation of frozen sections. Presence of epithelial dysplasia or tumor cells in the surgical resection margins is associated with a significant risk (66%) of local recurrence (3). However, even with histologically normal surgical margins, 10-30% of OSCC patients will still have local recurrence (4), which may lead to treatment failure and patient death.

Since histological status of surgical resection margins alone is not an independent predictor of local recurrence (5), histologically normal margins may harbor underlying genetic changes, which increase the risk of recurrence (6, 7). The prior art discloses candidate-gene approach studies that have identified genetic alterations in surgical resection margins in head and neck squamous cell carcinoma (HNSCC) from different disease sites, e.g., oral cavity, pharynx/hypopharynx, larynx (6-16). Genetic alterations identified in HNSCC included over-expression of elF4E (6, 9), TP53 (7, 11) and CDKN2A/P16 proteins (7). Other alterations reported included promoter hypermethylation of CDKN2A/P16 (13) and TP53 mutations (12, 16). In addition, promoter hypermethylation of CDKN2A, CCNAI and DCC was associated with decreased time to head and neck cancer recurrence (10).

Combined over-expression of COL4A1, encoding collagen type IV al chain and LAMC2, encoding laminin-γ2 chain, has been reported to distinguish OSCC from clinically normal oral tissues from individuals without head and neck cancer or preneoplastic oral lesions (28), and another study has reported differential expression between OSCC and normal mucosa, including MMP1, PLAU, MAGE-D4, GNA12, IFITM3 and NMU, regardless of aetiological factors (50).

### SUMMARY

Demonstrated herein is a molecular analysis of histologically normal surgical resection margins and their corresponding tumors to identify biomarkers involved in OSCC recurrence. A global gene expression analysis of histologically normal margins and their corresponding oral squamous cell carcinomas (OSCC) was performed, in conjunction with meta-analysis of public data, to identify 138 genes reliably up-regulated in OSCC (Table 4). A 4-gene signature optimized for prognostic value up-regulated in a subset of histologically normal was identified, and assessed for its clinical relevance and ability to predict recurrence in an independent cohort of patients with OSCC. In the independent validation cohort, all three gene subsets of this signature were also found to have some predictive value as were three of the four single genes and all but one of the two gene combinations (Table 8).

Accordingly, an aspect of the disclosure includes a method of diagnosing or predicting a likelihood of OSCC recurrence in a subject comprising:
a) determining an expression level of one or more biomarkers selected from Table 4 in a test sample from the subject, and
b) comparing the expression level of the one or more biomarkers with a control, wherein a difference or a similarity in the expression level of the one or more biomarkers between the test sample and the control is used to diagnose or predict the likelihood of OSCC recurrence in the subject.

Another aspect of the disclosure includes a method of diagnosing or predicting a likelihood of OSCC recurrence in a subject comprising:
a) determining an expression level of one or more biomarkers selected from MMP1, COL4A1, THBS2, and P4HA2, and optionally at least one of PXDN and/or PMEPA1, in a test sample from the subject, the one or more biomarkers comprising at least one of THBS2 and P4HA2, and
b) comparing the expression level of the one or more biomarkers with a control, wherein a difference or a similarity in the expression level of the one or more biomarkers between the test sample and the control is used to diagnose or predict the likelihood of OSCC recurrence in the subject.

In an embodiment, an increase the expression level of the one or more biomarkers between the test sample and the control is indicative or predictive of an increased likelihood of OSCC recurrence in the subject.

In another aspect, the disclosure includes a method of predicting a recurrence of OSCC in a subject comprising:
a) determining a subject biomarker expression profile from a test sample of the subject;
b) providing one or more biomarker reference expression profiles associated with OSCC recurrence and/or associated with survival without OSCC recurrence, wherein the subject biomarker expression profile and the biomarker reference expression profile(s) have a plurality of values, each value representing an expression level of a biomarker selected from the biomarkers in Table 4;
c) identifying the biomarker reference profile most similar to the subject biomarker expression profile,
   wherein the subject is predicted to have an increased likelihood of recurrence if the subject biomarker expression profile is most similar to the biomarker reference expression profile associated with OSCC recurrence and is predicted to have an decreased likelihood of recurrence if the subject biomarker expression profile is most similar to the biomarker reference expression profile associated with survival without OSCC recurrence.

In an embodiment, the biomarker expression profile comprises values for the expression level of at least 2 biomarkers.

In another aspect, the disclosure includes a method of predicting a recurrence of OSCC in a subject comprising:
a) determining a subject biomarker expression profile from a test sample of the subject;
b) providing one or more biomarker reference expression profiles associated with OSCC recurrence and/or associated with survival without OSCC recurrence, wherein the subject biomarker expression profile and the biomarker reference expression profile(s) have a plurality of values, each value representing an expression level of a biomarker selected from the biomarkers MMP1, COL4A1, THBS2, and P4HA2, and optionally at least one of PXDN and/or PMEPA1;
c) identifying the biomarker reference profile most similar to the subject biomarker expression profile,
   wherein the subject is predicted to have an increased likelihood of recurrence if the subject biomarker expression profile is most similar to the biomarker reference expression profile associated with OSCC recurrence and is predicted to have an decreased likelihood of recurrence if the subject biomarker expression profile is most similar to the biomarker reference expression profile associated with survival without OSCC recurrence.

In an embodiment, the method comprises obtaining a test sample from the subject for determining an expression level of the biomarkers.

In an embodiment, the method comprises calculating a risk score for comparison to the control. In another embodiment, the risk score calculation comprises summing a weighted expression level for one or more biomarkers, optionally wherein the weighted expression level comprises multiplying the relative expression level by a coefficient. In an embodiment, the coefficient is the coefficient in Table 6.

In yet another aspect, the disclosure includes a method of treating a subject in need thereof comprising:
a) obtaining a test sample from the subject;
b) predicting the likelihood of recurrence of OSCC in a subject according to any method described herein; and
c) administering to the subject predicted to have an increased likelihood of OSCC recurrence a treatment suitable for OSCC to increase survival without recurrence.

In a further aspect still, the disclosure provides a composition comprising at least two biomarker specific reagents that can detect or be used to determine the expression level of a biomarker selected from Table 4, optionally a biomarker selected from THBS2, P4HA2, COL4A1 and MMP1, and optionally at least one of PXDN or PMEPA1, wherein at least one biomarker is THBS2 or P4HA2.

In an embodiment, the composition comprises a plurality of isolated polynucleotides, such as at least two isolated polynucleotides, each isolated polynucleotide hybridizing to:
a) a RNA product of a biomarker selected from Table 4; and/or MMP1, COL4A1, THBS2, P4HA2, PXDN and/or PMEPA1,; and
b) a nucleic acid complementary to a),
wherein the composition is used to measure the level of RNA expression of the selected biomarkers.

In a further aspect, the disclosure includes an array comprising, for each of a plurality of biomarkers selected from Table 4, for example MMP1, COL4A1, THBS2, and P4HA2, and optionally PXDN and PMEPA1; one or more polynucleotide probes complementary and hybridizable to an expression product of the biomarker.

In yet another aspect, the disclosure includes a kit for predicting a likelihood of OSCC recurrence in a subject, comprising at least one biomarker specific agent that can detect or be used to determine the expression level of a biomarker selected from Table 4 such as THBS2, P4HA2, COL4A1 and MMP1; and a kit control.

In an embodiment, at least one of the biomarkers is THBS2 or P4HA2.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be described in relation to the drawings in which:

FIG. 1 is a protein-protein interaction network of 138 genes. I2D version 1.72 was used to identify protein interactions for the 138 genes shown in the heatmap. The resulting network was visualized using NAViGaTOR 2.1.14 (http://ophid.utoronto.ca/navigator). The shading of nodes corresponds to Gene Ontology biological function, as described in the legend. Highlighted squares represent the four genes in the signature of OSCC recurrence.

FIG. 2 is a heatmap of 138 genes up-regulated in OSCC. Expression values for each row (gene) are scaled to z-scores for visualization. Margins and tumors annotated with darker shading above the heatmap are from patients who experienced recurrence.

FIG. 3 is a heatmap of validation data and Kaplan-Meier plot of disease recurrence. (A) Unsupervised hierarchical clustering of the quantitative real-time PCR (validation data) showing the maximum expression levels of MMP1, P4HA2, THBS2 and COL4A1 in margins from patients with and without recurrence and with a follow-up time ≧12 months. Margins annotated with darker grey (labeled “Margin.recur”) above the heatmap are from patients who experienced recurrence. Margins from patients with locally recurrent tumors show increased expression levels of the four-gene signature compared to patients who did not recur. (B) Kaplan-Meier plot of quantitative real-time PCR data for patients in the validation set. Patients are assigned to high or low-risk based on their four-gene signature risk score. As seen in the Kaplan-Meier plot, patients with over-expression of the 4-gene signature are at high risk for disease recurrence; all patients who experienced recurrence in the validation set are in the high risk group, suggesting that over-expression of this signature was highly predictive of recurrence in the validation set. (C) Heatmap of validation data from unsupervised hierarchical clustering of the quantitative real-time PCR.

FIG. 4 is a bootstrap validation of four-gene signature risk score in training and validation sets. Density lines represent the distribution of hazard ratios observed in 1,000 re-samplings of a single margin, randomly chosen, from each patient.

FIG. 5 is a Bioanalyzer assessment of RNA integrity. Representative examples of RNA integrity results after Bioanalyzer assessment of paired fresh-frozen (upper) and FFPE (lower) samples. The fresh-frozen sample shown in the upper panel had an RIN=8.7 and the FFPE sample shown in the lower panel had a RIN=2.3.

FIG. 6 is a Correlation of results obtained from Nanostring analysis of paired fresh-frozen and FFPE tissues. Scatter plot matrix (left panel, A) for the normalized mRNA transcript quantification values obtained by Nanostring analysis of 19 fresh-frozen vs. FFPE sample pairs (n=38 samples). In this analysis, the pair-wise Pearson product-moment correlation coefficient was 0.90 (p<0.0001). The right panel (B) shows a heatmap analysis for the Pearson correlation of absolute mRNA transcript abundance as determined by Nanostring, for all pair-wise combinations of samples. These results show a good-high correlation between absolute mRNA transcript quantification data in fresh-frozen vs. FFPE tissues using Nanostring analysis. Fresh-frozen and FFPE tissues are interspersed, and all technical replicates are adjacent in all cases. Gene expression patterns are highly consistent among the large majority of samples.

FIG. 7 is a Correlation of results obtained from RQ-PCR analysis of paired fresh-frozen and FFPE tissues. Scatter plot matrix (left panel, A) showing normalized gene expression data obtained by RQ-PCR analysis of the 19 fresh-frozen vs. FFPE sample pairs (n=38 samples). The pair-wise Pearson product-moment correlation coefficient was 0.50 (p<0.0001). The right panel (B) shows a heatmap analysis for the Pearson correlation of gene expression abundance as determined by RQ-PCR, for all pair-wise combinations of samples. A low-moderate correlation is observed between mRNA transcript quantification data in fresh-frozen vs. FFPE tissues, and tissues tend to cluster according to storage method.

FIG. 8 is a Correlation between data obtained from Nanostring and RQ-PCR analysis on fresh-frozen and FFPE tissues. Scatter-plot matrices examining the correlation between Nanostring and RQ-PCR data in fresh-frozen (A) and FFPE (B) samples. Scatter plot matrices show normalized quantification values. The pair-wise Pearson product-moment correlation coefficient for Nanostring vs. RQ-PCR data in fresh-frozen samples was r=0.78 (p<0.0001); this same analysis revealed a lower correlation coefficient in FFPE samples (r=0.59) (p<0.0001). A corresponding heatmap for the Pearson correlation of gene expression abundance in fresh-frozen (FF) and FFPE samples using Nanostring vs. RQ-PCR is shown to the right of each scatter plot (C and D respectively). These results show a good correlation between Nanostring and RQ-PCR in fresh-frozen samples, and a lower correlation between data obtained using these two different technologies, when using clinical, archival, FFPE tissues. Table 1 lists the patient clinical data for the training set, in which 89 samples (histologically normal margins, OSCC and adjacent normal oral tissues) from 23 patients were used for oligonucleotide microarray analysis.

FIG. 9 demonstrates smoothed dependence of recurrence hazard on the four-gene risk score, calculated using the smoothCoxph function of the phenoTest R package (v1.2.0). Solid line gives log hazard ratio, and dashed lines indicate the 80% confidence interval.

FIG. 10 demonstrates smoothed dependence of recurrence hazard on each element of the four-gene risk score, calculated using the smoothCoxph function of the phenoTest R package (v1.2.0). Solid line gives log hazard ratio, and dashed lines indicate the 80% confidence interval. From left to right, then top to bottom: A) COL4A1, B) MMP1, C) P4HA2, and D) THBS2.

Table 1 lists the patient clinical data for the training set, in which 89 samples (histologically normal margins, OSCC and adjacent normal oral tissues) from 23 patients were used for oligonucleotide microarray analysis.

Table 2 lists the patient clinical data for the validation set, in which 136 samples (histologically normal margins, OSCC and adjacent normal oral tissues) from an independent cohort of 30 patients were used for quantitative RT-PCR (qRT-PCR) validation analysis.

Table 3 lists the four genes of the four-gene biomarker signature, the control gene, GAPDH, and the primer sequences used to validate the four-gene signature by qRT-PCR.

Table 4 lists 138 up-regulated genes in OSCC after data mining of the meta-analysis of public datasets and the in-house microarray experiment described in Example 1 below. For each gene, the raw p-value for univariate association with recurrence is given (logrank test), as well as false discovery rate (Benjamini Hochberg correction). Genes with false discovery rate (FDR) less than 0.3 may be valuable for prediction of recurrence. Several genes were subsequently tested for their ability to predict recurrence. The reduction from whole-genome to these 138 genes was not based on recurrence, so this validated the hypothesis that over-expressed genes can be used to predict recurrence based on expression levels in surgical margins.

Table 5 lists a subset of genes identified by Gene Ontology (GO) enrichment analysis of the 138 up-regulated genes.

Table 6 lists the coefficients of the linear risk score for z-score normalized log 2-expression values. Fold-change (FC) is the geometric-average expression in tumors relative to surgical resection margins. P-values are for tumor/margin differential expression in the qPCR (independent validation set) (Wilcoxon Rank Sum test).

Table 7 lists the sequence identifiers and accession numbers of the amino acid and polynucleotide sequences for MMP1, COL4A1, P4HA2, THBS2, PXDN and PMEPA1.

Table 8 lists the predictive ability of all subsets of the four-gene signature in the training and validation cohorts, estimated by bootstrap resampling of a single margin per patient. For each simulation, a single margin from each patient was selected randomly and used to calculate the risk score for that patient. These risk scores were used to estimate a hazard ratio for each simulation. Median HR is the median hazard ratio of the thousand simulations, and fraction >1 is the fraction of simulations where the estimated hazard ratio was greater than 1 (some predictive effect). Only two subsets in the validation set were not estimated to have predictive value (COL4A1 and THBS2+COL4A1). [

Table 9 lists the probe sequences used for digital molecular barcoding technology.

Table 10 lists accession numbers and SEQ ID NOs of exemplary amino acid and nucleic acid sequences of MMP1, COL4A1, P4HA2, THBS2, PXDN and PMEPA1.

Table 11 is a list of probe sets for genes of interest used for Nanostring analysis.

Table 12 is a list of primer sequences used in the RQ-PCR experiments.

### DESCRIPTION OF VARIOUS EMBODIMENTS

### I. Definitions

The term “antibody” as used herein is intended to include monoclonal antibodies, polyclonal antibodies, and chimeric antibodies. The antibody may be from recombinant sources and/or produced in transgenic animals.

The term “antibody binding fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies may be monospecific, bispecific, trispecific or of greater multispecificity. Multispecific antibodies may immunospecifically bind to different epitopes of a NADPH oxidase polypeptide and/or or a solid support material. Antibodies may be from any animal origin including birds and mammals (e.g., human, murine, donkey, sheep, rabbit, goat, guinea pig, camel, horse, or chicken).

Antibodies may be prepared using methods known to those skilled in the art. Isolated native or recombinant polypeptides may be utilized to prepare antibodies. See, for example, Kohler et al. (1975) Nature 256:495-497; Kozbor et al. (1985) J. Immunol. Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies.

In aspects, the antibody is a purified or isolated antibody. By “purified” or “isolated” is meant that a given antibody or fragment thereof, whether one that has been removed from nature (isolated from blood serum) or synthesized (produced by recombinant means), has been increased in purity, wherein “purity” is a relative term, not “absolute purity.” In particular aspects, a purified antibody is 60% free, preferably at least 75% free, and more preferably at least 90% free from other components with which it is naturally associated or associated following synthesis.

The term “biomarker” or “biomarker associated with oral squamous cell carcinoma recurrence” or “biomarkers of the disclosure” as used herein refer to a gene or genes, set out in Table 4 which have an FDR less than 0.3, and/or set out in Tables 3, 5 and/or 7 whose expression level in histologically normal tissue is associated with recurrence and/or an expression product (e.g. polypeptide or nucleic acid transcript) of such a gene, for example, a P4HA2, THBS2, COL4A1, or MMP1 and/or PXDN or PMEPA1 RNA transcript wherein the expression level in normal tissue is associated with recurrence. For example, it is demonstrated herein that increased expression levels combinations of 1 or more of P4HA2, THBS2, COL4A1, and/or MMP1 in tissue adjacent to OSCC (e.g. surgical resection margins) in a subject is associated with an increased recurrence of OSCC.

The phrase “biomarker polypeptide”, “polypeptide biomarker” or “polypeptide product of a biomarker” refers to a proteinaceous biomarker gene product which levels of are associated with recurrence of OSCC.

The phrase “biomarker nucleic acid”, or “nucleic acid product of a biomarker” refers to a polynucleotide biomarker gene product e.g. prognostic transcripts which levels of are associated with recurrence of OSCC.

The term “biomarker specific reagent” as used herein refers to a reagent that is a highly sensitive and specific for quantifying levels of a biomarker expression product, for example a polypeptide biomarker level or a nucleic acid biomarker product and can include antibodies which can for example be used with immunohistochemistry (1HC), ELISA and protein microarray or polynucleotides such as primers and probes which can for example be used with quantitative RT-PCR techniques, to detect the expression level of a biomarker associated with OSCC.

The term “classifying” as used herein refers to assigning, to a class or kind, an unclassified item. A “class” or “group” then being a grouping of items, based on one or more characteristics, attributes, properties, qualities, effects, parameters, etc., which they have in common, for the purpose of classifying them according to an established system or scheme. For example, subjects having an expression level of one or more biomarkers comprising at least one of THBS2 or P4HA2 as selected from the biomarkers listed in Table 4 with an FDR of less than 0.3, Table 3, 5 and/or 7 or a risk score calculated using the expression levels of the one or more biomarkers, above a threshold determined from the expression levels or weighted expression levels of control subjects can be predicted to have an increased likelihood of recurrence of oral small cell carcinoma. For example, subjects having increased expression of MMP1, COL4A1, THBS2, and/or P4HA2 in a test sample compared to a control are predicted to have a high-risk of recurrence of oral small cell carcinoma.

The term “coefficient” as related to biomarkers of the disclosure means a factor by which the expression, for example, the relative expression of each gene can be multiplied to provide a weighted expression level, for example using the coefficients provided in Table 6. The weighted expressions can for example be summed to calculate a risk score. For example, an increased expression level of a biomarker or biomarkers with a positive coefficient (e.g. increased compared to a control value such as a median value for a population of control subjects) is associated with an increased risk of OSCC recurrence and death.

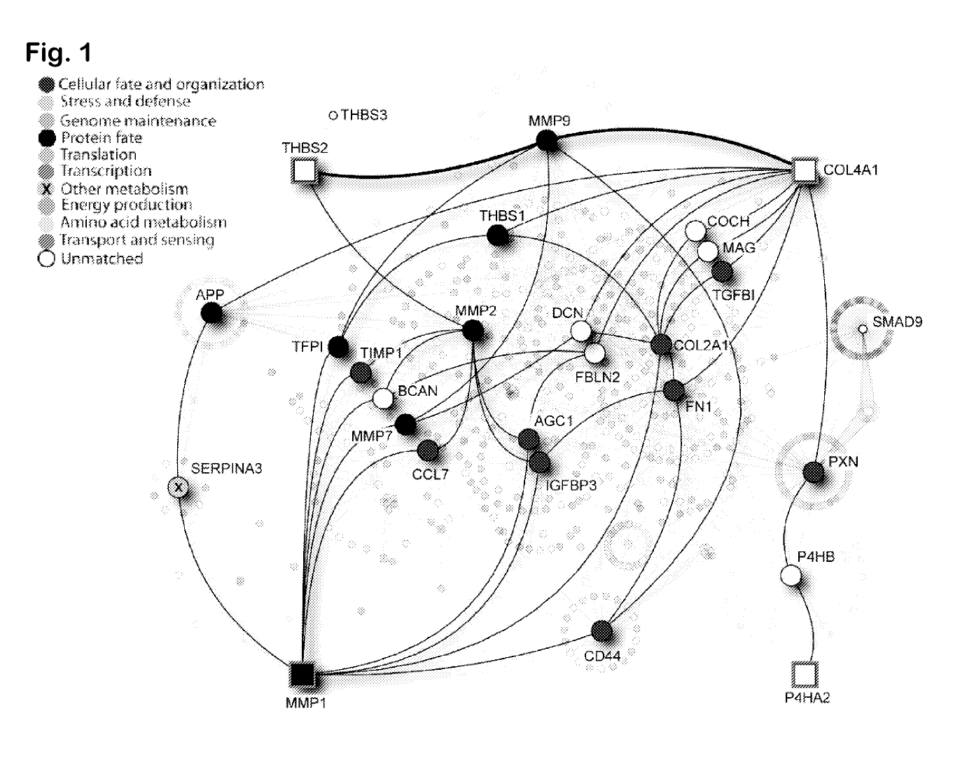

The term “COL4A1” as used herein refers to Collagen, type IV, alpha 1 which is the major type IV alpha collagen chain and includes without limitation all known COL4A1 molecules, preferably human, including naturally occurring variants, preferably human COL4A1 and including those deposited in Genbank with Entrez Gene ID accession number(s) 1282, Nucleotide ID number NM_{—}001845 and Swissprot ID numbers P02462, A7E2W4, B1AM70, Q1P9S9, Q5VWF6, Q86X41, Q8NF88, and Q9NYC5, as described for example in Table 4, and which are each herein incorporated by reference as well as the nucleic acid sequence of SEQ ID NO:13 and/or the amino acid of sequence of SEQ ID NO:14, as described in Table 10. COL4A1 binds other collagens (COL4A2, 3, 4, 5 and 6), as well as LAMC2 (laminin, gamma 2), TGFB1 (transforming growth factor, beta 1), among other proteins (FIG. 1) (http://www.ihop-net.org), playing a relevant role in extracellular matrix-receptor interaction and focal adhesion (26).

The term “control” as used herein refers to a sample or samples of normal oral tissue, or a fraction thereof such as but not limited to, normal oral tissue RNA or normal oral tissue protein, and/or a biomarker level or biomarker levels, numerical value and/or range (e.g. control range) corresponding to the biomarker level or levels in such a sample or samples (e.g. average, median, cut-off value etc). The normal oral tissue sample can for example be taken from a subject or a population of subjects (e.g. control subjects) who are known as not having OSCC and/or not having cancer (e.g. healthy individuals). Alternatively, the control can be adjacent normal tissue that is for example taken at least 2 cm or at least 3 cm distal to any cancer for example from any OSCC lesion or former OSCC lesion site (e.g. not comprising a surgical margin). Adjacent normal tissue may be taken for example from the patient being assessed (e.g. test sample and control sample from the same patient). The normal oral tissue can be for example, any normal tissue from the oral cavity of healthy individuals known not to have an oral cancer. This can include for example normal oral tissue of the same tissue type as the test sample (e.g. a tissue type matched control). Alternatively, the control can be a numerical value corresponding to and/or derived from the expression level of one or more biomarkers in normal oral tissue that is predetermined.

Where the control is a numerical value or range, the numerical value or range is a predetermined value or range that corresponds to a level of the biomarker or biomarkers or range of the biomarker(s) in normal oral tissue of a group of subjects known as not having OSCC (e.g. threshold or cutoff level; or control range) or corresponding to adjacent normal oral tissue at least 2 cm away from any cancer including any OSCC lesion or former lesion or for example corresponding to histologically normal tissue (including for example surgical margins) for a subject or subjects known to have long term survival without recurrence. Alternatively, the cut-off can be the median expression level of one or more biomarkers in the histologically normal resection margins of a population of subjects, resected for OSCC. For example it is demonstrated herein that biomarker expression levels that are below the median expression level in histologically normal resection margins in a population of subjects, are associated with long-term survival without recurrence. For example, the control can be a selected cut-off or threshold level, or control score comprising for example a desired specificity above which a subject is identified as having an increased likelihood of developing OSCC recurrence, e.g. corresponding to a median level in a population. For example, a test subject that has an increased level of a biomarker or biomarkers above a cut-off, threshold level or control score is indicated to have or is more likely to have recurrence of OSCC.

The cut-off, threshold or control score can for example be a median level or value, or composite score comprising the median expression level or levels, for example the weighted expression levels, in a population of subjects. Following a larger clinical study, this threshold can be determined to optimize the trade-off between false negative and false positive discoveries, for example by optimizing the area under the ROC curve. It may also be desirable to define multiple thresholds, for example to assign patients to high, medium, and low risk groups. The threshold(s) may be at any percentile of risk scores in the study sample, for example corresponding to the lowest 90%, 80%, 70%, 60%, 50%, 40%, 30%, 20% or 10% of risk scores calculated form histologically normal margins in a population of subjects. A person skilled in the art would understand that “control” as herein defined is distinct from for example a PCR control, no template PCR control or internal control, which is used for example with quantitative PCR. For example an internal control is a nonbiomarker gene that is expected to be expressed at relatively the same level in different samples that is used to quantify the relative amount of biomarker transcript for comparison purposes.

The term “control level” refers to a biomarker level in a control sample or a numerical value corresponding to such a sample. Control level can also refer to for example a threshold, cut-off or baseline level of a biomarker for example in subjects without OSCC, where levels above which are associated with an increased likelihood of OSCC recurrence.

The term “determining an expression level” or “determining an expression profile” as used in reference to a biomarker means the application of a biomarker specific reagent such as a probe, primer or antibody and/or a method to a sample, for example a sample of the subject and/or a control sample, for ascertaining or measuring quantitatively, semi-quantitatively or qualitatively the amount of a biomarker or biomarkers, for example the amount of biomarker polypeptide or mRNA. For example, a level of a biomarker can be determined by a number of methods including for example immunoassays including for example immunohistochemistry, ELISA, Western blot, immunoprecipation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR, serial analysis of gene expression (SAGE), Northern Blot, digital molecular barcoding technology, for example Nanostring nCounter™ Analysis, and TaqMan quantitative PCR assays (see Example 6 for further details). Other methods of mRNA detection and quantification can be applied, such as mRNA in situ hybridization in formalin-fixed, paraffin-embedded (FFPE) tissue samples or cells. This technology is currently offered by the QuantiGene® ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and this technology has been shown to be useful for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The term “diagnosing or predicting recurrence of OSCC” refers to a method or process of assessing the likelihood that a subject will or will not have recurrence of oral squamous cell carcinoma based on biomarker expression levels of biomarkers associated with recurrence.

The term “difference in the level” as used herein in comparison to a control refers to a measurable difference in the level or quantity of a biomarker or biomarkers associated with OSCC recurrence in a test sample, compared to the control that is of sufficient magnitude to allow assessment of the likelihood of recurrence, for example a significant difference or a statistically significant difference. The magnitude of the difference is sufficient for example to determine that the subject falls within a class of subjects likely to have OSCC recurrence or likely to have long-term survival without recurrence. For example, the difference can be a difference in the steady-state level of a gene transcript or translation product, including for example a difference resulting from a difference in the level of transcription and/or translation and/or degradation that is sufficient to distinguish with acceptable specificity whether a subject is likely to have or not have an OSCC recurrence. A sufficient difference is for example a level or risk score that is statistically associated with a particular group or outcome, for example having recurrence of OSCC or not having recurrence OSCC. For example, a difference in a level of biomarker level is detected if a ratio of the level in a test sample as compared with a control is greater than 1.2. For example, a ratio of greater than 1.5, 1.7, 2, 3, 3, 5, 10, 12, 15, 20 or more.

The term “digital molecular barcoding technology” as used herein refers to a digital technology that is based on direct multiplexed measurement of gene expression that utilizes color-coded molecular barcodes, and can include for example Nanostring nCounter™. For example, in such a method each color-coded barcode is attached to a target-specific probe, for example about 50 bases to about 100 bases or any number between 50 and 100 in length that hybridizes to a gene of interest. Two probes are used to hybridize to mRNA transcripts of interest: a reporter probe that carries the color signal and a capture probe that allows the probe-target complex to be immobilized for data collection. Once the probes are hybridized, excess probes are removed and detected. For example, probe-target complexes can be immobilized on a substrate for data collection, for example an nCounter™ Cartridgeand analysed for example in a Digital Analyzer such that for example color codes are counted and tabulated for each target molecule. Further details are provided for example in Example 6.

The term “expression level” as used herein in reference to a biomarker refers to a quantity of biomarker that is detectable or measurable in a sample and/or control. The quantity is for example a quantity of polypeptide, or a quantity of nucleic acid e.g. biomarker transcript. Accordingly, a polypeptide expression level refers to a quantity of biomarker polypeptide that is detectable or measurable in a sample and a nucleic acid expression level refers to a quantity of biomarker nucleic acid that is detectable or measurable in a sample.

The term “expression profile” as used herein refers to, for one or a plurality (e.g. at least two) of biomarkers that are associated with OSCC recurrence, biomarker steady state and/or transcript or polypeptide expression levels in a sample from a subject. For example, an expression profile can comprise the quantitated relative levels of at least one or more biomarkers comprising at least one of THBS2 or P4HA2 as selected from the biomarkers listed in Table 4 with a FDR of less than 0.3, and/or Table 3, 5 and/or 7, and the levels or pattern of biomarker expression can be compared to one or more reference profiles, for example a reference profile associated with recurrence of OSCC and/or a reference profile associated with survival without recurrence. The plurality optionally comprises at least 2, at least 3, at least 4, at least 5, or more of the 138 genes listed in Table 4 and/or the genes described in Example 6, including for example any number of genes between 2 and 138.

The term “histologically normal margins” or “histologically normal surgical resection margins” as used herein refers to the histological status of cells and/or tissue from the surgical resection margins from patients with OSCC. Histologically normal cells, tissue, and/or resection margins as referred to herein lack the presence of epithelial dysplasia or tumor cells.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, hybridization in 6.0× sodium chloride/sodium citrate (SSC) at about 45° C., followed by a wash of 2.0×SSC at 50° C. may be employed.

The term an “increased likelihood of recurrence” or “high-risk of recurrence”, as used herein means that a test subject who has increased levels of one or more biomarkers, for example comprising at least one of THBS2 or P4HA2 as selected from the biomarkers listed in Table 3 and/or 7 and/or one or more biomarkers listed in Table 5, and/or one or more biomarkers listed in Table 4 with a FOR of less than 0.3 (i.e. FDR<0.3) has an increased chance of OSCC recurrence in less than for example 24 months, 18 months, 12 months, or 8 months after surgery and consequently poor survival relative to a control subject (e.g. a subject with control levels of one or more of the biomarkers listed in Table 4 and/or 5; and/or Table 3 and/or 7 biomarkers comprising at least one of THBS2 or P4HA2). The increased risk for example may be relative or absolute and may be expressed qualitatively or quantitatively. For example, an increased risk may be expressed as simply determining the test subject's expression level for a given biomarker and placing the test subject in an “increased risk” category, based upon previous population studies. Alternatively, a numerical expression of the test subject's increased risk may be determined based upon biomarker level analysis. For example a risk score can be calculated. Conversely “decreased likelihood of recurrence or “low-risk of recurrence” as used herein means that a test subject who has normal levels of the biomarkers listed in Table 3 and/or 7 and/or Table 5, and/or the biomarkers listed in Table 4 with a FDR of less than 0.3 (i.e. FDR<0.3) has an increased chance of long term survival without recurrence, for example survival without recurrence for at least 12 months, 18 months, or 24 months. In embodiments where subjects are classified as high, moderate or low risk; “moderate risk” is defined as having a risk score above the “low risk” threshold but below the “high risk” threshold. Optimal values for these thresholds can be estimated from the current data. As used herein, examples of expressions of a risk include but are not limited to, hazard ratio, odds, probability, odds ratio, p-values, attributable risk, relative frequency, and relative risk. The relationship between hazard of recurrence and overexpression of the four gene signature in histologically normal margins is described for example in Example 7.

The term “kit control” as used herein means a suitable assay control useful when determining an expression level of a biomarker associated with OSCC recurrence. For example, for kits for determining polypeptide biomarker levels, the kit control optionally comprises a biomarker polypeptide (or peptide fragment) that can for example be used to prepare a standard curve or act as a positive antibody control. Alternatively, the kit control is an antibody to a non-biomarker polypeptide such as actin for determining relative biomarker levels. For kits for detecting RNA levels for example by hybridization, the kit control can comprise an oligonucleotide control, useful for example for detecting an internal control such as GAPDH for standardizing the amount of RNA in the sample and determining relative biomarker transcript levels. The kit control can also comprise one or more control oligonucleotides that can be used to detect transcript levels of control genes, for example, one or more housekeeping genes, for example, genes with constant expression in oral tissues.

The term “MMP1” as used herein means Matrix Metalloprotease 1, and includes without limitation all known MMP1 molecules, preferably human, including naturally occurring variants, including for example MMP1 transcript variant 1 and MMP1 transcript variant 2, and including those deposited in Genbank with Entrez Gene ID accession number(s) 4312, Nucleotide ID number NM_{—}002421, and Swissprot protein ID numbers P03956 and P08156, for example as described in Table 4, and which are each herein incorporated by reference as well as the nucleic acid sequence of SEQ ID NO:11 and/or the amino acid sequence of SEQ ID NO:12, as described in Table 10. MMP1 is a key collegenase, secreted by tumor cells as well as stromal cells stimulated by the tumor, involved in extracellular matrix (ECM) degradation (29). MMP1 is responsible for breaking down interstitial collagens type I, II and III in normal physiological processes (e.g., tissue remodeling) as well as disease processes (e.g., cancer) (29). It is believed that the mechanism of up-regulation of most of the MMPs is likely due to transcriptional changes, which may occur following alterations in oncogenes and/or tumor suppressor genes (29). MMP1 is mapped on 11q22.3 of the human chromosome.

The term “measuring” or “measurement” as used herein refers to assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The term “oral squamous cell carcinoma” or “OSCC” as used herein refers to a subtype of head and neck cancers that includes squamous cell carcinomas of the oral cavity. The squamous cell carcinomas of the oral cavity can affect, for example, tongue, floor of the mouth, palate, alveolus, cheek (or buccal), and gingival tissue. All stages and metastasis are included.

The term “P4HA2” as used herein means prolyl 4-hydroxylase, alpha polypeptide II and includes without limitation all known P4HA2 molecules, preferably human including naturally occurring variants, for example P4HA2 transcript variant 1, P4HA2 transcript variant 2, P4HA2 transcript variant 3, P4HA2 transcript variant 4, and P4HA2 transcript variant 5, and including those deposited in Genbank with Entrez Gene ID accession number(s) 8974; Nucleotide ID numbers NM_{—}004199 (variant 1), NM_{—}001017973 (variant 2), NM_{—}001017974 (variant 3), NM_{—}001142598 (variant 4), and NM_{—}001142599 (variant 5); and Swissprot protein ID numbers O15460 and Q8WWN0, which are described for example in Table 4, and which are each herein incorporated by reference, as well as the nucleic acid sequence of SEQ ID NO:15, the amino acid sequence of SEQ ID NO:16 and/or the amino acid sequence of SEQ ID NO:17, as described in Table 10. P4HA2 refers to a key enzyme involved in collagen synthesis, whose over-expression has been previously reported in papillary thyroid cancer (23). P4HA2 gene is mapped on chromosome 5q31.1 of the human, and has regulatory transcription factor binding sites in its promoter regions.

The term “PMEPA1” as used herein means prostate transmembrane protein, androgen induced 1 and includes without limitation all known PMEPA1 molecules, preferably human, including naturally occurring variants, for example PMEPA1 transcript variant 1, PMEPA1 transcript variant 2, PMEPA1 transcript variant 3, and PMEPA1 transcript variant 4, and including those deposited in Genbank with Entrez Gene ID accession number(s) 56937; Nucleotide ID numbers NM_{—}020182.3 (variant 1), NM_{—}199169 (variant 2), NM_{—}199170 (variant 3), and NM_{—}199171 (variant 4); and Swissprot protein ID numbers Q969W9, Q5TDR6, Q96B72, and Q9UJD3, which are described for example in Table 4 and which are each herein incorporated by reference, as well as the nucleic acid sequence of SEQ ID NO:20 and/or the amino acid sequence of SEQ ID NO:21, as described in Table 10.

The term “PXDN” as used herein means Peroxidasin homologand includes without limitation all known PXDN molecules, preferably human, including naturally occurring variants, and including those deposited in Genbank with Entrez Gene ID accession number(s) 7837, Nucleotide ID number NM_{—}012293, and Swissprot protein ID numbers Q92626, A8QM65, and Q4KMG2, which are described for example in Table 4 and which are each herein incorporated by reference as well as the nucleic acid sequence of SEQ ID NO:22 and/or the amino acid sequence of SEQ ID NO:23, as described in Table 10.

The term “polynucleotide”, “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded, represent the sense or antisense strand.

The term “primer” as used herein refers to a polynucleotide, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

The term “probe” as used herein refers to a nucleic acid sequence that will hybridize to a nucleic acid target sequence. In one example, the probe hybridizes to a biomarker RNA or a nucleic acid sequence complementary to the biomarker RNA. The length of probe depends for example, on the hybridization conditions and the sequences of the probe and nucleic acid target sequence. The probe can be for example, at least 15, 20, 25, 50, 75, 100, 150, 200, 250, 400, 500 or more nucleotides in length.

A person skilled in the art would recognize that “all or part of a particular probe or primer can be used as long as the portion is sufficient for example in the case a probe, to specifically hybridize to the intended target and in the case of a primer, sufficient to prime amplification of the intended template.

The term “risk” as used herein refers to the probability that an event will occur over a specific time period, for example, as in the recurrence of OSCC within 12, 18, or 24 months after surgery, in a subject diagnosed and surgically treated for OSCC and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

The term “recurrence” or “OSCC recurrence” as used herein means development of OSCC after an interval in a subject diagnosed and treated for OSCC, for example development of OSCC post treatment, for example post surgical resection. Recurrence can include, for example, local recurrence of a cancer near the primary site of resection and/or distal recurrence.

The term “recurrence risk score” or “risk score” as used herein refers to a sum of the weighted biomarker expression levels for one or more of the biomarkers listed in Table 3 and/or 7 and/or Table 5 and/or the biomarkers listed Table 4 with an FDR<0.3, optionally wherein at least one of the biomarkers is THBS2 or P4HA2. The risk score is calculated on the basis of coefficients such as the coefficients in Table 6. Coefficients can be for example, determined in a large prospective trial, using the methods described herein, for example using Nanostring or qPCR as described for example in the Examples below.

The term “reference expression profile” as used herein refers to a suitable comparison profile, for example a polypeptide or nucleic acid reference profile that comprises the level of one or more biomarkers selected from the biomarkers listed in Table 3 and/or 7 and/or Table 5 and/or the biomarkers listed Table 4 with an FDR<0.3, optionally wherein at least one of the biomarkers is THBS2 or P4HA2, in normal oral tissue of a subject or population of subjects, for example in a subject or subjects optionally expression levels corresponding to surgical margin tissue from a subject or subjects who later recur (e.g. expression profile associated with OSCC recurrence) or corresponding to surgical margin tissue from a subject or subjects who have long term survival without recurrence (e.g. greater than 12, 18, or 24 without recurrence). For example, the “reference expression profile” can be a RNA expression profile or a polypeptide profile. As the expression products of nucleic acid transcripts, polypeptide levels can be expected to correspond to nucleic acid transcript levels, for example mRNA levels, The reference expression profile is an expression signature (e.g. polypeptide or nucleic acid gene expression levels and/or pattern) of a one or a plurality of genes (e.g. at least 2 genes, for example 4 genes), associated for example with OSCC recurrence or long-term survival without recurrence. The reference expression profile is accordingly a reference profile or reference signature of the expression of one or more biomarkers selected from the biomarkers listed in Table 3 and/or 7 or the biomarkers listed Table 4 with an FDR<0.3, optionally wherein at least one of the biomarkers is THBS2 or P4HA2 to which the expression levels of the corresponding genes in a test sample are compared in methods for example for determining recurrence of OSCC.

The term “sample” as used herein refers to any oral biological fluid, cell or tissue or fraction thereof from a subject that can be assessed for biomarker expression products, polypeptide expression products or nucleic acid expression products, including for example an isolated RNA fraction, optionally mRNA for nucleic acid biomarker determinations and a protein fraction for polypeptide biomarker determinations. A “test sample” comprises histologically normal oral tissue (or a fraction thereof e.g. RNA or protein fraction) proximal to an OSCC lesion or proximal to a former OSCC lesion, for example within up to 1.9 cm of a tumor edge. The histologically normal tissue can be taken by biopsy (e.g. prior to surgical resection) or during surgical resection or following surgical resection The histologically normal tissue can for example be buccal, floor of the mouth (FOM), tongue, alveolar, retromolar, palate, gingival, or other oral tissue; and/or tissue from margins adjacent to tumor resection. A “control sample” comprises normal oral tissue (or a fraction thereof such as isolated RNA, optionally mRNA or a protein fraction) corresponding to a subject or subjects without OSCC or corresponding to normal oral tissue at least 2 cm distal to the edge of any tumor, including any OSCC or former tumor. The sample for example can comprise formalin fixed and/or paraffin embedded tissue, a frozen tissue or fresh tissue. The sample can be used directly as obtained from the source or following a pretreatment to modify the character of the sample, e.g. to obtain a RNA or polypeptide fraction. Where the control is RNA, the control RNA can also be referred to as reference RNA. Reference RNA can include for example a universal RNA pool.

The term “sequence identity” as used herein refers to the percentage of sequence identity between two or more polypeptide sequences or two or more nucleic acid sequences that have identity or a percent identity for example about 70% identity, 80% identity, 90% identity, 95% identity, 98% identity, 99% identity or higher identity or a specified region. To determine the percent identity of two or more amino acid sequences or of two or more nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, word_length=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted.

The term “similar” in the context of a biomarker level as used herein refers to a subject biomarker level that falls within the range of levels associated with a particular class for example associated with recurrence of oral squamous cell carcinoma or associated with long-term survival without recurrence (e.g. similar to a control level). Accordingly, “detecting a similarity” refers to detecting a biomarker level that falls within the range of levels associated with a particular class. In the context of a reference profile, “similar” refers to a reference profile associated with recurrence or long-term survival without recurrence of oral squamous cell carcinoma that shows a number of identities and/or degree of changes with the subject expression profile.

The term “most similar” in the context of a reference profile refers to a reference profile that shows the greatest number of identities and/or degree of changes with the subject expression profile.

The term “specifically binds” as used herein refers to a binding reaction that is determinative of the presence of the biomarker (e.g. polypeptide or nucleic acid) often in a heterogeneous population of macromolecules. For example, when the biomarker specific reagent is an antibody, specifically binds refers to the specified antibody binding with greater affinity to the cognate antigenic determinant than to another antigenic determinant, for example binds with at least 2, at least 3, at least 5, or at least 10 times greater specificity; and when a probe, specifically binds refers to the specified probe under hybridization conditions binds to a particular gene sequence at least 1.5, at least 2 at least 3, or at least 5 times background.

The term “subject” as used herein refers to any member of the animal kingdom, preferably a human being.

The term “THBS2” as used herein refers to thrombospondin 2 and includes without limitation all known THBS2 molecules, preferably human, including naturally occurring variants, and including those deposited in Genbank with Entrez Gene ID accession number(s) 7058, Nucleotide ID number NM_{—}003247, and Swissprot protein ID number P35442, described for example in Table 4, and which are each herein incorporated by reference, as well as the nucleic acid sequence of SEQ ID NO:18 and/or the amino acid sequence SEQ ID NO:19, as described in Table 10. THBS2 is a matricellular protein that encodes an adhesive glycoprotein and interacts with other proteins to modulate cell-matrix interactions (24). Interestingly, THBS2 is associated with tumor growth in adult mouse tissues (24). THBS2 may modulate the cell surface properties of mesenchymal cells, is involved in cell adhesion and migration and binds to collagen 4. THBS2 is mapped on chromosome 6q27 of the human chromosome.

The phrase “therapy” or “treatment” as used herein, refers to an approach aimed at obtaining beneficial or desired results, including clinical results and includes medical procedures and applications including for example chemotherapy, pharmaceutical interventions, surgery, radiotherapy and naturopathic interventions as well as test treatments for treating oral squamous cell carcinoma. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

Moreover, a “treatment” or “prevention” regime of a subject with a therapeutically effective amount of the compound of the present disclosure may consist of a single administration, or alternatively comprise a series of applications.

The term “treatment suitable for a subject with OSCC” refers to a treatment that is suitable for a patient or subject with OSCC, including early stage OSCC or a pre-OSCC condition. For example, detection of increased expression of one or more of the biomarkers can be indicative of early molecular changes prior to OSCC detection (e.g. a pre-OSCC condition) that can lead to OSCC recurrence. Accordingly, the treatment can be one that is suitable for treating such a pre-condition. Treatments suitable can include for example radiation treatment, for example adjuvant post-operative radiation treatment.

The term “tumor resection margins” or “surgical margins” or “surgical resection margins” as used herein refers to tissue excised proximal to and/or that immediately surrounds tumor tissue, for example within up to 1.9 cm of a tumor edge. For example when tumor tissue is surgically removed or resected, tissue is excised to ensure no tumor is left behind in the patient. The tissue excised proximal to the tumor can, for example, be histologically normal (or histologically negative) or can contain dysplasia or even some tumor cells (histologically positive). Only patients with histologically normal tumor margins were assessed in the present studies, which can also be referred to as “histologically normal tumor margins”. One or more margins can be analysed, as the tumor is three dimensional, normal tissue can be present surrounding the tumor.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies:

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps. For example, the phrase “one or more biomarkers does not consist of THBS2 and COL4A1” or “the at least one biomarker does not consist of THBS2 and COL4A1” or other similar phrases as used herein means that the biomarkers cannot be a group of two biomarkers that are THBS2 and COL4A1, but can be any other combination of biomarkers.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### II. Methods and Apparatus

### A. Diagnostic Methods

The genetic alterations identified to date have not been used clinically in the assessment of surgical margins, and a gene signature that can accurately predict which patients with oral squamous cell carcinoma (OSCC) are at a higher risk of disease recurrence has not been developed.

The lack of definitive predictive biomarkers may be caused by most studies treating HNSCCs from distinct anatomic sites as one tumor type. Although the current histopathological classification of these tumors classifies them under one heading; clinically, they may behave differently at distinct sites, suggesting underlying biological differences. Furthermore, high-throughput analysis of multiple surgical margins and matched OSCCs to identify deregulated genes predictive of recurrence has not been used.

It is demonstrated herein that tumor-like molecular changes found in histologically normal resection margins are biomarkers associated with OSCC recurrence. These changes precede histological alteration and provide more accurate prediction of recurrence in patients with OSCC.

A number of biomarkers whose expression is elevated in OSCC tumors were assessed for their association with OSCC recurrence and are listed in Table 4. Biomarkers with a FDR of for example less than 0.3 may be useful for prognosing recurrence.

Accordingly, an aspect of the disclosure includes a method of diagnosing or predicting a likelihood of OSCC recurrence in a subject comprising:

a) determining an expression level of one or more biomarkers selected from Table 4 in a test sample from the subject, and

b) comparing the expression level of the one or more biomarkers with a control, wherein a difference or a similarity in the expression level of the one or more biomarkers between the test sample and the control is used to diagnose or predict the likelihood of OSCC recurrence in the subject.

In another aspect, the disclosure includes a method of predicting a recurrence of OSCC in a subject comprising:

a) determining a subject biomarker expression profile from a test sample of the subject;

b) providing one or more biomarker reference expression profiles associated with OSCC recurrence and/or associated with survival without OSCC recurrence, wherein the subject biomarker expression profile and the biomarker reference expression profile(s) have one or a plurality of values, each value representing an expression level of a biomarker selected from the biomarkers in Table 4;
c) identifying the biomarker reference profile most similar to the subject biomarker expression profile,
   wherein the subject is predicted to have an increased likelihood of recurrence if the subject biomarker expression profile is most similar to the biomarker reference expression profile associated with OSCC recurrence and is predicted to have an decreased likelihood of recurrence if the subject biomarker expression profile is most similar to the biomarker reference expression profile associated with survival without OSCC recurrence.

In an embodiment, the biomarkers are selected from the biomarkers listed in Table 4 with an FDR<0.3, for example, the biomarkers are selected from THBS2, MMP1, COL4A1, PXDN, P4HA2, PMEPA1, COL5A2, SERPINH1, COL5A1, CTHRC1, COL3A1, SERPINE2, PLOD2, POSTN, COL4A2, COL1A2, COL1A1, PDPN, TNC, SERPINE1, MFAP2, MMP10, TLR2, C4orf48, GREM1, C9orf30, FAP, and EGFL6.

Table 5 comprises a subset of the markers in Table 4. In an embodiment, the biomarkers are selected from the subset in Table 5. Table 3 lists four biomarkers of a four gene signature. In an embodiment, the biomarkers are selected from the subset in Table 4. Table 7 lists THBS2, MMP1, COL4A1, PXDN, P4HA2, PMEPA1. In another embodiment, the biomarkers are selected from the subset in Table 7.

Further, a multi-step procedure including meta-analysis of published microarray datasets and a whole-genome expression profiling experiment was used to develop a 4-gene prognostic signature for OSCC recurrence, which is described herein. The signature is based on genes found to be over-expressed in tumors as compared to normal tissues and the majority of histologically normal surgical resection margins. Over-expression of this 4-gene signature in tumor resection margins provides an early indication of genetic changes before histological alterations can be detected by histopathological examination. The prognostic ability of the gene signature was validated by quantitative real-time PCR (qRT-PCR) in an independent cohort of 30 patients (Hazard Ratio (HR)=6.8, p=0.04). The maximum expression level of each gene in the tumor resection margins was calculated for each patient in the independent cohort, and was used to calculate the risk score for each patient. Using the median risk score determined in the training set, the patients were split into high and low-risk groups (15 patients in each). The high-risk group contained six of the seven recurrences and suffered a significantly higher rate of recurrence (HR=6.8, p=0.04 log-rank test). Therefore, the 4-gene signature can be used to detect tumor-like gene expression alterations to predict OSCC recurrence, which can be used for example, for patients with histologically normal surgical resection margins.

The genes identified in the four-gene signature (MMP1, COL4A1, THBS2 and P4HA2) play major roles in cell-cell and/or cell-matrix interaction, and invasion. The direct and indirect partners of these genes are illustrated in FIG. 1. The changes in these four genes provide for more accurate prediction of recurrence in patients who have had OSCC.

One, two and three subset combinations of the four gene signature were assessed for OSCC prognostic ability. Table 8, demonstrates that combinations of 1, 2, and 3 biomarkers have prognostic ability for predicting recurrence.

Accordingly, an aspect of the disclosure includes a method of predicting a likelihood of OSCC recurrence in a subject comprising:

a) determining an expression level of one or more biomarkers selected from MMP1, COL4A1, THBS2 and P4HA2 in a test sample from the subject, the one or more biomarkers comprising at least one of THBS2 and P4HA2, and

b) comparing the expression level of the one or more biomarkers with a control,

wherein a difference or a similarity in the expression level of the one or more biomarkers between the test sample and the control is used to predict the likelihood of OSCC recurrence in the subject.

In an embodiment, the biomarkers assessed do not consist of the set THBS2 and COL4A1. While subsets of 1, 2, 3 and 4 genes of the biomarkers were shown to be indicative of recurrence, an increase in expression level of COL4A1 alone and COL4A1 and THBS2 did not show significant predictive value (Table 8). In an embodiment, the combination of biomarkers comprises at least one of the biomarkers THBS2 or P4HA2 and one or more of COL4A1 and MMP1.

In another embodiment, an increase in the level in at least one of the biomarkers THBS2 or P4HA2 is indicative of an increased likelihood of recurrence of OSCC.

In an embodiment, the test sample comprises tissue from histologically normal margins for example from an OSCC surgical resection.

In embodiment, one or more samples are assessed, for example each sample comprising a distinct histologically normal surgical margin biopsy.

In an embodiment, the expression level is a maximal biomarker expression level of the one or more samples is compared to the control.

In an embodiment, the expression level is a relative expression level or a log ratio.

In another embodiment, the expression level of the one or more biomarkers is used to calculate a risk score for the subject, wherein the risk score calculation comprises summing a weighted expression level for each of the one or more biomarkers determined in the test sample.

In another embodiment, the risk score is compared to a control, wherein the control is a predetermined threshold and/or is calculated by adding a weighted expression level for each of the one or more biomarkers in a control or corresponding to a control population of subjects.

For example, a subject is identified as having an increased risk of recurrence based on a multivariate linear risk score with a pre-defined cutoff between high and low risk, when the subject's risk score is above the pre-defined cutoff. Prediction is currently based on a multivariate linear risk score with a pre-defined cutoff between high and low risk.

In an embodiment, the weighted expression level comprises the relative expression level multiplied by a coefficient specific for the biomarker, optionally a coefficient in Table 6.

In another embodiment, comparing the expression level of the one or more biomarkers in the test sample with a control comprises determining the relative expression of each biomarker compared, calculating a risk score for the subject, and using the risk score to classify the subject as having a high-risk or a low risk of recurrence of OSCC, or optionally as having a high-risk, moderate-risk or a low-risk of recurrence of OSCC by comparing the risk score to a threshold score or scores.

In an embodiment, the subject is predicted to have a high risk of recurrence when the risk score is greater than the control.

In an embodiment, the threshold score is a score comprising the median, or corresponding to the lowest 50%, 40%, 30%, 20% or 10% expression levels in histologically normal oral tissue in a population of subjects (e.g. control population).

The relationship between hazard of recurrence and over-expression of the four-gene signature in histologically normal margins is discussed in Example 7. A sensitivity analysis using the quantitative PCR data was done to demonstrate the relationship between hazard of recurrence and over-expression of each gene. The strength of association is shown to be different for each gene, being strongest for P4HA2 and MMP1. For example for P4HA2 and MMP1, a 50% increase in expression could confer a substantial increased risk of recurrence (˜5-fold), and for COL4A1 and THBS2 a 2-fold increase produces a comparable increase in risk. For example, a 50% increase in P4HA2 and MMP1. or a 50% increase in any of these genes in combination with a 2-fold increase in COL4A1 and THBS2 would suggest an increased risk of recurrence.

Accordingly in an embodiment, the increase in expression of one or more of the biomarkers is at least 10%, at least 20%, at least 30%, at least 40%, at least 50%, at least 60%, at least 70%, at least 80%, at least 90%, at least 100%, at least 1.5fold, at least 2 fold, at least 3 fold, at least 4 fold or at least 5 fold increased compared to a control.

In an embodiment, the sample being tested is compared to a control sample (e.g standard normal sample, for example tongue tissue from healthy individuals or a universal RNA pool could be used as the control sample (e.g. reference RNA sample) for PCR. The margin sample could be compared for example to a predetermined range established for example from a clinical trial.

In an embodiment, the relative expression of each gene in for example the four-gene signature would be calculated from quantitative PCR−Ct (Cycle threshold) values. Ct values are used in an algorithm—the delta delta Ct method (69) to determine relative gene expression. These values would be used to calculate the combined risk score by a weighted average (e.g. Table 6). The values of the risk score can be used in conjunction with a pre-established table to look up risk of recurrence based on the patents' score. For example, patients can be considered “high risk” if their risk score is above the median risk score determined from the training set (score=0.2), and “low risk” if their score is below this threshold. In this example, “high risk” patients in the validation set are 7 times more likely to experience recurrence (95% Cl=0.8−58, Wald Test) than “low risk” patients (see for example Example 7).

Determining the likelihood of recurrence of oral squamous cell carcinoma may involve classifying a subject with OSCC based on the similarity or difference of the subject's expression profile to an expression profiles associated with OSCC recurrence or long term survival without recurrence. A high likelihood of recurrence of OSCC in a subject can alter clinical management decisions, which in turn can lead to improved individualized patient treatment and improved survival. In this sense, more accurate prediction is especially important when about 30% of OSCC patients with histologically normal surgical resection margins recur.

In another aspect, the disclosure includes a method of predicting a recurrence of OSCC in a subject comprising:

a) determining a subject biomarker expression profile from a test sample of the subject;

b) providing one or more biomarker reference expression profiles associated with OSCC recurrence and/or associated with long term survival without OSCC recurrence, wherein the subject biomarker expression profile and the biomarker reference expression profile(s) have one or a plurality of values, each value representing an expression level of a biomarker selected from the biomarkers MMP1, COL4A1, THBS2 and/or P4HA2, and optionally at least one of PXDN or PMEPA1;
c) identifying the biomarker reference profile most similar to the subject biomarker expression profile,
   wherein the subject is predicted to have an increased likelihood of recurrence if the subject biomarker expression profile is most similar to the biomarker reference expression profile associated with OSCC recurrence and is predicted to have an decreased likelihood of recurrence if the subject biomarker expression profile is most similar to the biomarker reference expression profile associated with survival without OSCC recurrence.

In an embodiment, the biomarkers comprises at least one or both of PXDN or PMEPA1.

In another embodiment, the biomarkers further comprise at least one or more of the biomarkers listed in Table 4 with an FDR<0.3. In an embodiment, the one or more biomarkers further comprises at least one or more of the biomarkers listed in Table 5. In another embodiment, the one or more biomarkers further comprises at least one or more of the biomarkers listed in Table 3 or 7.

In an embodiment, the expression level of at least 2, at least 3 or 4 of MMP1, COL4A1, THBS2 and P4HA2 is determined and compared. In another embodiment, the biomarkers do not consist of THBS2 and COL4A1.

As mentioned, in another embodiment, biomarkers are selected from the biomarkers listed in Table 4 with an FDR<0.3. In another embodiment, the biomarkers further comprise at least one or more of COL5A2, SERPINH1, COL5A1, CTHRC1, COL3A1, SERPINE2, PLOD2, POSTN, COL4A2, COL1A2, COL1A1, PDPN, TNC, SERPINE1, MFAP2, MMP10, TLR2, C4orf48, GREM1, C9orf30, FAP, and EGFL6.

In an embodiment, the expression of level or expression profile of, at least 2, at least 3, at least 4, at least 5, at least 6, at least 8, at least 10 or more biomarkers is determined and compared to the control. In an embodiment, the one or more biomarkers comprises at least 5, at least 10, at least 15 or at least 20 of the biomarkers selected from biomarkers in Table 4 and/or 5.

In an embodiment, an increase in the expression levels of one or more biomarkers is indicative of recurrence. In an embodiment, an increase in the expression of level of at least 1, at least 2, at least 3, at least 4 or more of the biomarkers compared to the control is indicative of an increased likelihood of recurrence of OSCC in the subject.

Similarity can be assessed for example by determining if the similarity between an expression profile and a reference profile is above or below a predetermined threshold.

Accordingly, in another embodiment, the method comprises:

a) calculating a measure of similarity between an expression profile and one or more reference expression profiles, the expression profile comprising the expression levels of a first plurality of biomarkers in a sample taken from the subject; the one or more reference expression profiles associated with recurrence or associated with long-term survival without recurrence comprising, for each biomarker of the plurality, the average or median expression level of the gene in a population of subjects associated with the reference expression profile; the plurality of biomarkers comprising two or more of the biomarkers listed in Tables 3, 4, 5 and/or 7; and

b) classifying the subject as having an increased likelihood of recurrence if the expression profile has a high similarity to the reference expression profile associated with recurrence or has a higher similarity to the reference expression profile associated with recurrence than to the reference expression profile associated with long term survival without recurrence or classifying the subject as having an increased likelihood of long term survival without recurrence if the expression profile has a low similarity to the reference expression profile reference expression profile associated with recurrence or has a higher similarity to the reference expression profile associated with long term survival without recurrence than to the reference expression profile associated with recurrence; wherein the expression profile has a high similarity to the reference expression profile associated with recurrence if the similarity to the reference profile associated with recurrence is above a predetermined threshold, or has a low similarity to the reference profile associated with recurrence if the similarity to the reference expression profile associated with recurrence is below the predetermined threshold.

In an embodiment of the disclosure, the biomarker expression level determined is a nucleic acid level.

In another embodiment, determining the biomarker expression level or expression profile comprises amplification of the biomarker transcript(s) for example by using a PCR based technique including for example, quantitative PCR, such as quantitative RT-PCR, or comprises use of one or more of serial analysis of gene expression (SAGE), in situ hybridization, microarray, digital molecular barcoding technology such as nanostring nCounter, or Northern Blot or other probe based analysis. In an embodiment, the expression level is determined using qPCR and/or digital molecular barcoding technology such as nanostring nCounter.

As described in Example 6, SYBR Green I fluorescent dye-based RQ-PCR and NanoString nCounter™ assays can be used for gene expression analysis including for example of archival oral carcinoma samples, such as archival, formalin-fixed, paraffin embedded (FFPE) samples and fresh-frozen samples. It is demonstrated therein that the genes composing the four-gene signature (MMP1, COL4A1, P4HA2, THBS2,) were which were included among the 20 genes tested showed that both technologies (Nanostring, probe-based assay, and QPCR are useful to detect and measure gene expression levels in formalin-fixed, paraffin embedded samples. The probe-based assay dd achieved superior gene expression quantification results in FFPE samples compared to QPCR.

Example 6 determines the mRNA transcript abundance of 20 genes (COL3A1, COL4A1, COL5A1, COL5A2, CTHRC1, CXCL1, CXCL13, MMP1, P4HA2, PDPN, PLOD2, POSTN, SDHA, SERPINE1, SERPINE2, SERPINH1, THBS2, TNC, GAPDH, RPS18) in 38 samples (19 paired fresh-frozen and FFPE oral carcinoma tissues, archived from 1997-2008) by both NanoString and SYBR Green I fluorescent dye-based quantitative real-time PCR(RQ-PCR). As demonstrated therein, the gene expression data obtained by NanoString vs. RQ-PCR was compared in both fresh-frozen and FFPE samples. Fresh-frozen samples showed a good overall Pearson correlation of 0.78, and FFPE samples showed a lower overall correlation coefficient of 0.59, which is likely due to sample quality. A higher correlation coefficient was observed between fresh-frozen and FFPE samples analyzed by NanoString (r=0.90) compared to fresh-frozen and FFPE samples analyzed by RQ-PCR (r=0.50). In addition, NanoString data showed a higher mean correlation (r=0.94) between individual fresh-frozen and FFPE sample pairs compared to RQ-PCR (r=0.53).

Both of these technologies can be used for gene expression quantification in fresh-frozen or FFPE tissues. As demonstrated, the probe-based NanoString method achieves superior gene expression quantification results when compared to RQ-PCR in archived FFPE samples.

In an embodiment, determining the biomarker expression level comprises amplification of the biomarker nucleic acid expression level or expression profile using a nucleic acid primer that hybridizes to a biomarker nucleic acid transcript. In an embodiment, the nucleic acid comprises all or part of any one of SEQ ID NOs:1 to 8. In an embodiment, determining the biomarker expression comprises using a primer, selected from any one of SEQ ID NOs: 1 to 8 of a primer pair, wherein at least of one or two primer(s) of the primer pair is selected from SEQ ID NOs:1 to 8.

In another embodiment, determining the biomarker expression level comprises amplification of the of the biomarker nucleic acid expression level or expression profile using a nucleic acid primer that hybridizes to a biomarker transcript. In an embodiment, the method comprises using a primer or primer pair selected from the primers listed in Table 12. In an embodiment the primer pair is selected from SEQ ID NOs:52 and 53; SEQ ID NOs:54 and 55; SEQ ID NOs: 58 and 59 and/or SEQ ID NOs: 78 and 79.

In an embodiment, the one or more biomarkers comprises MMP1 and the expression level of MMP1 is determined using a primer comprising at least one of SEQ ID NO:1 SEQ ID NO:2, SEQ ID NO:52 and SEQ ID NO:53, optionally SEQ ID NO:1 and SEQ ID NO:2 and/or SEQ ID NO: 52 and SEQ ID NO:53. In another embodiment, the one or more biomarkers comprises COL4A1 and the expression level of COL4A1 is determined using a primer comprising at least one of SEQ ID NO:3, SEQ ID NO:4, SEQ ID NO:54 and SEQ ID NO:55, optionally SEQ ID NO:3 and SEQ ID NO:4 and/or SEQ ID NO: 54 and SEQ ID NO:55. In a further embodiment, the one or more biomarkers comprises THBS2 and the expression level of THBS2 is determined using a primer comprising at least one of SEQ ID NO:5, SEQ ID NO:6 SEQ ID NO: 58 and SEQ ID NO:59, optionally SEQ ID NO:5 and SEQ ID NO:6 and/or SEQ ID NO: 58 and SEQ ID NO:59. In yet a further embodiment, wherein the one or more biomarkers comprises P4HA2 and the expression level of P4HA2 is determined using a primer comprising at least one of SEQ ID NO:7, SEQ ID NO:8 SEQ ID NO: 78 and SEQ ID NO:79, optionally SEQ ID NO:7 and SEQ ID NO:8 and/or SEQ ID NO: 78 and SEQ ID NO:79.

In an embodiment, determining the biomarker expression level comprises using an array.

In another embodiment, determining the biomarker expression level comprises using digital molecular barcoding technology using a nucleic acid probe that hybridizes to a biomarker transcript nucleic acid. In an embodiment, the nucleic acid probe comprises at least 10, at least 15 at least 20, at least 30, at least 40, at least 50, at least 60, at least 70, at least 80 or at least 90 or more contiguous nucleotides of any one of SEQ ID NOs:24 to 27. In an embodiment, determining the biomarker expression level comprises using a probe, selected from any one of SEQ ID NOs: 24 to 27. In another embodiment, the method comprises using at least 10, at least 15 at least 20, at least 30, at least 40, at least 50, at least 60, at least 70, at least 80 or at least 90 or more contiguous nucleotides nucleic acid probes described in Table 11. In an embodiment, the method comprises using at least 10, at least 15 at least 20, at least 30, at least 40, at least 50, at least 60, at least 70, at least 80 or at least 90 or more contiguous nucleotides of one or more of the probes of SEQ ID NOs: 35, 29, 44 and 36. The probe can be for example from about 10 to about 100 contiguous nucleotides, or any number of nucleotides in between.

In an embodiment, the one or more biomarkers comprises MMP1 and the expression level of MMP1 is determined using a probe comprising SEQ ID NO:24 and/or SEQ ID NO:35. In another embodiment, the one or more biomarkers comprises COL4A1 and the expression level of COL4A1 is determined using a probe comprising SEQ ID NO:25 and/or SEQ ID NO:29. In a further embodiment, the one or more biomarkers comprises P4HA2 and the expression level of P4HA2 is determined using a probe comprising SEQ ID NO:26 and/or SEQ ID NO:36. In yet a further embodiment, the one or more biomarkers comprises THBS2 and the expression level of THBS2 is determined using a probe comprising SEQ ID NO:27 and/or SEQ ID NO: 44.

In yet another embodiment, the expression level of the biomarker determined is a polypeptide level. In still another embodiment, determining the biomarker expression level or profile comprises using an antibody specific for the biomarker polypeptide. In yet another embodiment still, determining the biomarker level comprises assaying the polypeptide level by immunohistochemistry, Western blot or array.

As indicated in Example 7, polypeptide levels typically correlate to nucleic acid transcript levels. Accordingly, antibody-based methods for detection of proteins could also be used for predicting the risk of recurrence. In this method, immunohistochemical analysis can be employed using specific antibodies to detect the presence and/or level of biomarker gene products, for example for the four genes in the signature.

In an embodiment, the sample comprises an oral tissue sample. In an embodiment, the sample is a biopsy. In another embodiment, the sample is a surgical biopsy, removed for example during an OSCC resection. In an embodiment, the biopsy is a punch biopsy, for example a 2 mm punch biopsy. In another embodiment, the test sample comprises histologically normal tumor resection margin tissue. In a further embodiment, the control is derived from normal oral tissue, for example from a subject or subjects without OSCC. In still another embodiment, the oral tissue sample comprises buccal mucosa or cheek, FOM, tongue, alveolar, palate, gingival or retromolar tissue. In a further embodiment, the test sample and the control are derived from the same tissue type, e.g. the test sample comprises resection margins from a buccal OSCC to determine biomarker expression levels and the control corresponds to normal buccal tissue biomarker levels. In an embodiment, the sample comprises formalin fixed and/or paraffin embedded tissue, a frozen tissue or fresh tissue.

In an embodiment, the method comprises determining the expression level in several fractions of a test sample.

In an embodiment, the average expression level of the biomarker in the plurality of samples is compared. In another embodiment, the maximum expression level is compared.

### B. Methods of Treatment

More accurate prediction of recurrence of oral squamous cell carcinoma (OSCC) can be useful in aiding clinical management decisions, leading to improved individualized treatment. Accordingly, an aspect of the disclosure includes a method of treating a subject in need thereof comprising:

a) predicting the likelihood of recurrence of OSCC in the subject according to any of the methods disclosed herein; and

b) administering to a subject predicted to have an increased likelihood of OSCC recurrence, a treatment suitable for OSCC or a pre OSCC condition.

In an embodiment, a suitable treatment is administered in the absence of other clinical and histopathological indicators of OSCC in the subject, for example to prevent or inhibit recurrence. A suitable treatment can include radiation treatment. In an embodiment, the radiation is adjuvant post-operative radiation treatment.

For example, once the recurrence risk is determined looking at histologically normal margins, adjuvant radiation treatment can be performed as well as closer follow-up to monitor patients for disease recurrence.

In an embodiment, the method comprises providing and/or obtaining a sample obtained from the subject, e.g. to determine an expression level of one or more biomarkers of the disclosure.

### C. Methods of Identifying a Signature

The methods described herein for determining a signature useful for predicting or classifying the likelihood of recurrence of oral squamous cell carcinoma (OSCC) can be used to identify signatures for identifying likelihood of recurrence of other cancers and/or other diseases.

For example, the methods herein identify a signature using global gene expression analysis (for example by microarrays) of surgical margins. Previous studies have analyzed surgical resection margins and oral cancers; however, these studies have done so using only candidate gene approaches. Analysis of surgical resection margins has not been performed using global gene expression analysis.

Accordingly, another aspect of the disclosure includes a method of identifying a biomarker signature associated with a high-risk of recurrence of a cancer in the absence of histological changes, the method comprising:

a) using global gene expression analysis to identify a subset of genes that are over-expressed in tumors relative to normal tissues or adjacent normal tissue, optionally resection margins from publicly available datasets of the cancer;

b) identifying a subset of genes that are over-expressed in a separate set of tumor samples relative to adjacent normal tissue, optionally resection margins;

c) creating a list of genes that are over-expressed in the cancer based on the intersection of the genes of a) with b);

d) subjecting the genes of c) to regression analysis, optionally a penalized Cox regression analysis; and

e) selecting the genes with the largest coefficients.

In an embodiment, the biomarker signature is validated using a leave one out method. In another embodiment, the biomarker signature is validated using qRT-PCR using for example primers that amplify a prognostic biomarker transcript of the biomarker signature.

In another embodiment, the global gene expression analysis comprises using microarrays.

A multi-step model of identifying a biomarker signature is described herein which can for example be applied to other cancers or cancer subtypes. In an embodiment, a first step comprises identifying genes that are overexpressed, for example at least two-fold over-expressed in tumors relative to normal tissues or adjacent normal tissue such as resection margins, optionally wherein the data is derived from publicly available datasets. In an embodiment, the proportion of false positives of these genes is set to a desired false discovery rate, for example set to less than 0.01 (i.e. False Discovery Rate or “FDR” of 0.01). In an embodiment, a second step comprises identifying genes that are over-expressed for example, at least two-fold over-expressed in a separate set of tumor samples relative to normal tissues, for example normal adjacent resection margins. In another embodiment, the expression levels are determined using microarray analysis.

In yet another embodiment, a third step comprises creating a list of genes that are over-expressed in the cancer based on the intersection of the identified genes, wherein the criteria of two-fold over-expression in tumors. In a further embodiment, a fourth step comprises subjecting the list of genes up-regulated in tumors to regression analysis such as a penalized Cox regression analysis, wherein the penalized Cox regression analysis. In an embodiment, the expression level of each gene is manipulated prior to the regression analysis, and the method comprises:

a) calculating a maximum expression level of the gene, for example where more than biopsy or repeat of a sample is taken; and

b) converting each maximum expression level of a) to a z-score for each gene before the regression analysis.

In yet another embodiment, the penalized Cox regression analysis further comprises selecting a penalty parameter. In another embodiment, the penalty parameter is selected by optimizing 10-fold cross-validated likelihood.

In another embodiment, a fifth step comprises selecting a subset of genes with the largest coefficients.

### D. Computer Implemented Methods

The methods described herein can be computer implemented. In an embodiment, the method further comprises: displaying or outputting to a user interface device, a computer readable storage medium, or a local or remote computer system, the classification produced by the classifying step disclosed herein; and/or an indication of the likelihood of recurrence or a value (such as a risk score) corresponding to the likelihood of recurrence. In another embodiment, the method comprises displaying or outputting a result of one of the steps to a user interface device, a computer readable storage medium, a monitor, or a computer that is part of a network.

### E. Compositions, Kits, Arrays and Computer Products

Another aspect of the disclosure includes a composition comprising at least two biomarker specific reagents that can detect or be used to determine the expression level of a biomarker selected from a biomarker listed in Table 3, 4, 5 and/or 7 for example THBS2, P4HA2, COL4A1 and MMP1, wherein at least one biomarker is THBS2 or P4HA2. In an embodiment, the biomarkers do not consist of THBS2 and COL4A1.

In an embodiment, the composition further comprises a biomarker specific reagent specific for at least one of PXDN or PMEPA1.

In another embodiment, the composition comprises a biomarker specific reagent specific for at least one or more of the biomarkers listed in Table 4 with an FDR<0.3. In another embodiment, the composition comprises a biomarker specific reagent specific for at least one or more of COL5A2, SERPINH1, COL5A1, CTHRC1, COL3A1, SERPINE2, PLOD2, POSTN, COL4A2, COL1A2, COL1A1, PDPN, TNC, SERPINE1, MFAP2, MMP10, TLR2, C4orf48, GREM1, C9orf30, FAP, and EGFL6.

In an embodiment, the composition comprises a plurality of isolated polynucleotides, such as at least two isolated polynucleotides, wherein each isolated polynucleotide hybridizes to:

a) a RNA product of a biomarker selected from Table 3, 4, 5 and/or 7 such as MMP1, COL4A1, THBS2, P4HA2, PXDN and PMEPA1, optionally wherein at least one of the biomarkers is THBS2 or P4HA2; or

b) a nucleic acid complementary to a),

wherein the composition is used to measure the level of RNA expression of one or more biomarkers associated with OSCC recurrence.

In one embodiment, the biomarker is at least 2, at least 3 or 4 of THBS2, P4HA2, MMP1 and COL4A1. In an embodiment the biomarkers comprise THBS2, P4HA2, MMP1 and COL4A1.

In another embodiment, the composition comprises one or more probes, primers, or primer sets. In an embodiment, the composition comprises one or more and all or part of any one of SEQ ID NO:1-8, or the SEQ ID NOs listed in Table 12, such as SEQ ID NOs: 52-55, 58-59 and 78-79. In another embodiment, the composition comprises one or more and all or part of any one of SEQ ID NO:24 to 27, 35, 29, 44 and 36.

In still another embodiment, the composition comprises all or part, for example at least 10 or at least 15 contiguous nucleotides of each of SEQ ID NO:5 and SEQ ID NO:6; and/or SEQ ID NO:7 and SEQ ID NO:8. In yet another embodiment, the composition comprises all or part of each of SEQ ID NO:1 and SEQ ID NO:2; SEQ ID NO:3 and SEQ ID NO:4; SEQ ID NO:5 and SEQ ID NO:6; and/or SEQ ID NO:7 and SEQ ID NO:8. In yet another embodiment, the composition comprises a primer set, optionally at least two, at least 3 or four of the pairs of SEQ ID NO:1 and SEQ ID NO:2, SEQ ID NO:3 and SEQ ID NO:4, SEQ ID NO:5 and SEQ ID NO:6, and/or SEQ ID NO:7 and SEQ ID NO:8. In an embodiment the composition comprises all or part, for example least 10 or at least 15 contiguous nucleotides of each of SEQ ID NO:58 and SEQ ID NO:59; and/or SEQ ID NO:78 and SEQ ID NO:79. In yet another embodiment, the composition comprises all or part of each of SEQ ID NO:52 and SEQ ID NO:53; SEQ ID NO:54 and SEQ ID NO:55; SEQ ID NO:58 and SEQ ID NO:59; and/or SEQ ID NO:78 and SEQ ID NO:79. In yet another embodiment, the composition comprises a primer set, optionally at least two, at least 3 or four of the pairs of SEQ ID NO:52 and SEQ ID NO:53, SEQ ID NO:54 and SEQ ID NO:55, SEQ ID NO:58 and SEQ ID NO:59, and/or SEQ ID NO:78 and SEQ ID NO:79.

In another embodiment, the composition comprises an internal control polynucleotide, for determining an expression level of a non-biomarker polynucleotide level, optionally wherein the control polynucleotide comprises SEQ ID NO:9 and/or SEQ ID NO:10; SEQ ID 48 and/or 49; and/or SEQ ID NO:50 and SEQ ID NO:51

In yet another embodiment, the composition comprises a diluent or carrier.

In an embodiment, the composition comprises all or part, for example at least 15, at least 20, at least 25, at least 30, at least 40, at least 50, at least 60 at least 70 at least 80, at least 90 or contiguous nucleotides, of each of SEQ ID NO:26 and/or SEQ ID NO:27; SEQ ID NO:36 and/or 44 In yet another embodiment, the composition comprises all or part of one or more or each of SEQ ID NO:24, SEQ ID NO:25, SEQ ID NO:26 SEQ ID NO:27 SEQ ID NO: 35; SEQ ID NO: 29, SEQ ID NO:44 and SEQ ID NO: 36. In yet another embodiment, the composition does not consist of all or part SEQ ID NO:25 and SEQ ID NO:27.

Another aspect of the disclosure includes an array comprising, for each of a plurality of biomarkers selected from Tables 4, 5 and/or 7 such as MMP1, COL4A1, THBS2, and P4HA2, and optionally PXDN and PMEPA1; one or more probes, optionally polynucleotide probes complementary and hybridizable to an expression product of the biomarker.

In an embodiment, the array comprises probes for detecting THBS2, P4HA2, MMP1 and COL4A1. In an embodiment, the array comprises polynucleotide probes.

Another aspect of the disclosure includes a kit for example to classify a subject with OSCC as having a high likelihood of recurrence or a low likelihood of recurrence.

In an embodiment, the kit comprises one or more of:

a) a composition described herein; and/or

b) a biomarker specific reagent described herein;

c) a kit control; and

d) instructions for use.

In another embodiment still, the kit further comprises reagents for qRT-PCR, including buffers, reverse transcription and amplification primers for the target genes and endogenous control genes, and control RNA from normal oral tissue.

In another embodiment, the kit further comprises reagents for digital molecular barcoding technology, including for example buffers, hybridization solution, and/or one or more labeled probes.

The kit can optionally comprise sample collection tubes and/or assay plates for conducting one or more assays.

In an embodiment, the kit comprises a kit control, and at least one biomarker specific agent that can detect or be used to determine an expression level of one or more biomarkers selected from biomarkers listed in Table 3, 4, 5 and/or 7 such as THBS2, P4HA2, COL4A1 and MMP1, wherein at least one biomarker is THBS2 or P4HA2. In an embodiment, the kit comprises at least 2, at least 3 or at least 4 biomarker specific agents.

In an embodiment, the kit comprises a biomarker specific agent that detects or can be used to determine the expression level of THBS2, P4HA2, MMP1 or COL4A1. In another embodiment, the kit comprises biomarker specific agents, which detect or be used to determine the expression level of at least two of THBS2, P4HA2, MMP1 or COL4A1. In yet another embodiment, the kit comprises biomarker specific agents which detect or can be used to determine the expression level of at least three of THBS2, P4HA2, MMP1 or COL4A1.

In another embodiment, the kit further comprises a biomarker specific agent that can detect or be used to determine the expression level of at least one or both PXDN and/or PMEPA1.

In another embodiment, the kit further comprises a biomarker specific agent that can detect or be used to determine the expression level of at least one or more of the biomarkers listed in Table 4 with an FDR<0.3. In another embodiment, the kit further comprises a biomarker specific agent that can detect or be used to determine the expression level of at least one or more of COL5A2, SERPINH1, COL5A1, CTHRC1, COL3A1, SERPINE2, PLOD2, POSTN, COL4A2, COL1A2, COL1A1, PDPN, TNC, SERPINE1, MFAP2, MMP10, TLR2, C4orf48, GREM1, C9orf30, FAP, and EGFL6.

In another embodiment, the biomarker specific agent is a probe, primer or primer set that amplifies a nucleic acid transcript of the biomarker. In yet another embodiment, the primer sets comprise at least one of a pair of SEQ ID NO:5 and SEQ ID NO:6 or SEQ ID NO:7 and SEQ ID NO:8; or SEQ ID NO:58 and SEQ ID NO: 59 or SEQ ID NO:36 and 37. In still another embodiment, the primer sets further comprise at least one of the pairs of SEQ ID NO:1 and SEQ ID NO:2, SEQ ID NO:3 and SEQ ID NO:4, SEQ ID NO:5 and SEQ ID NO:6, or SEQ ID NO:7 and SEQ ID NO:8; or SEQ ID NO: 52 and 53; SEQ ID NO: 54 and 55; SEQ ID NO 58 and 59.0r SEQ ID NO: 78 and 79 In yet another embodiment, the primer sets further comprise at least two of the pairs of SEQ ID NO:1 and SEQ ID NO:2, SEQ ID NO:3 and SEQ ID NO:4, SEQ ID NO:5 and SEQ ID NO:6, SEQ ID NO:7 and SEQ ID NO:8; SEQ ID NO: 52 and 53; SEQ ID NO: 54 and 55; SEQ ID NO 58 and 59.0r SEQ ID NO: 78 and 79. In another embodiment, the primer sets further comprise at least three of the pairs of SEQ ID NO:1 and SEQ ID NO:2, SEQ ID NO:3 and SEQ ID NO:4, SEQ ID NO:5 and SEQ ID NO:6, SEQ ID NO:7 and SEQ ID NO:8 SEQ ID NO: 52 and 53; SEQ ID NO: 54 and 55; SEQ ID NO 58 and 59.0r SEQ ID NO: 78 and 79.

In another embodiment, the probes comprise at least one of SEQ ID NO:26 or SEQ ID NO:27. In another embodiment, the probes comprise at least one of SEQ ID NO:35 or SEQ ID NO:29. In still another embodiment, the probes further comprise at least one of SEQ ID NO:24, SEQ ID NO:25, SEQ ID NO:26 SEQ ID NO:27, SEQ ID NO: 35, SEQ ID NOL 29, SEQ ID NO:44 and SEQ ID NO; 36. In yet another embodiment, the probes further comprise at least two of SEQ ID NO:24, SEQ ID NO:25, SEQ ID NO:26 SEQ ID NO:27. SEQ ID NO: 35, SEQ ID NOL 29, SEQ ID NO:44 and SEQ ID NO; 36. In another embodiment, the probes further comprise at least three of SEQ ID NO:24, SEQ ID NO:25, SEQ ID NO:26 SEQ ID NO:27 SEQ ID NO: 35, SEQ ID NOL 29, SEQ ID NO:44 and SEQ ID NO; 36. In still another embodiment, the probes do not consist of SEQ ID NO:25 and SEQ ID NO:27 or SEQ ID NO:29.

In another embodiment, the kit control is an RNA control such as reference RNA.

In an embodiment, the kit comprises reference RNA, PCR primers for the four-gene signature and optionally PCR primers for one or more housekeeping genes.

In another embodiment, the kit comprises a pre-determined recurrence of risk associated with different values of the risk score.

In an embodiment, the kit comprises an array comprising a plurality of biomarker detection agents for detecting one or more biomarkers listed in Table 3, 4, 5, and/or 7.

The kit can comprise for example, specimen collection tubes for example for collecting a biopsy, extraction buffer, positive controls, and the like.

A further aspect comprises a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method:
a) receive a value corresponding to an expression level of one or more biomarkers selected from the biomarkers listed in Table 3, 4, 5 and/or 7 in a test sample from the subject,
b) compare the value of each expression level of the one or more biomarkers in the test sample with a control; and
c) display a recurrence prediction and/or classification;
   wherein a difference or a similarity in the expression level of the one or more biomarkers between the control and the test sample is used to classify the recurrence status of the subject as having a high likelihood of recurrence or a low likelihood of recurrence.

In an embodiment, comparing the expression comprises determining the relative expression level of the one or more biomarkers, for example compared to the control sample and optionally an endogenous control gene (e.g., an internal control used for example in PCR based methods) and using the relative expression of each biomarker to calculate a value of the risk score of the subject using a weighted average given by coefficients in for example Table 6. The determination of recurrence status is for example made based on the value of the risk score compared to a threshold determined for a population of subjects with known outcome.

In an embodiment, the computer program product is for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the method:
a) receive a subject biomarker expression profile in a test sample of the subject;
b) compare the subject biomarker expression profile to one or more biomarker reference expression profiles, each biomarker reference expression profile associated with a recurrence or long-term survival without recurrence, wherein the subject biomarker expression profile and the each reference expression profile have a plurality of values each value representing an expression level of a biomarker selected from the biomarkers listed in Table 7;
c) select the biomarker reference expression profile most similar to the subject biomarker profile; and
d) display a recurrence prediction;
   wherein the subject is predicted to recur if the subject biomarker expression profile is most similar to the reference expression profile associated with recurrence and predicted to have long term survival without recurrence if the subject biomarker expression profile is most similar to the reference expression profile associated long term survival without recurrence.

Another aspect includes a computer implemented product for predicting a OSCC recurrence in a subject comprising:
a means for receiving values corresponding to a subject expression profile in a test sample; and
a database comprising a plurality of reference expression profiles each associated with a recurrence prognosis, wherein the subject biomarker expression profile and the biomarker reference expression profile each has a plurality of values, each value representing an expression level of a biomarker listed in Table 7;
   wherein the computer implemented product selects the reference expression profile most similar to the subject biomarker expression profile, to thereby predict a recurrence prognosis or classify the subject.

In an embodiment, the computer-implemented product is for use with a method described herein.

A further aspect is a computer readable medium having stored thereon a data structure for storing the computer-implemented product described herein.

In an embodiment, the data structure is capable of configuring a computer to respond to queries based on records belonging to the data structure, each of the records comprising:
a value that identifies a biomarker reference expression level of one or more biomarkers listed in Table 7;
a value that identifies the probability of recurrence associated with the biomarker reference expression level.

Also provided in an aspect is a computer system for predicting recurrence or classifying a subject comprising:
a) a database comprising a plurality reference expression profiles, each associated with a prognosis, wherein the subject biomarker expression profile and the biomarker reference expression profile each has a plurality of values, each value representing the expression level of a biomarker, wherein the biomarkers are selected from Table 7;
b) a server having computer-executable code for effecting the following steps;
   i. receiving a subject expression profile;
   ii. identifying from the database a reference expression profile that is most similar to the subject expression profile; and
   iii. outputting a descriptor of the reference expression profile identified.

In an embodiment, the descriptor is an associated recurrence prognosis. In another embodiment, the descriptor is a treatment associated with the reference expression profile. In another embodiment, the descriptor is transmitted across a network.

### III. Examples

### Example 1

### Methods

### Patients

This work was performed with the approval of the University Health Network Research Ethics Board. All patients signed their informed consent before sample collection, and were untreated before surgery. Tissue samples were obtained at time of surgery from the Toronto General Hospital, Toronto, Ontario, Canada. Primary OSCC and histologically normal margin samples were snap-frozen in liquid nitrogen until RNA extraction.

### Samples Used for Microarrays (Training Set)

89 samples (histologically normal margins, OSCC and adjacent normal tissues) from 23 patients were used for microarrays. An experienced head and neck pathologist (BP-O) performed histological evaluation of all surgical margins to ensure that they were histologically normal. No patient used in this study had a histologically positive margin. Patient clinical data for this training set are summarized in Table 1.

### Samples Used for Quantitative Real-Time Reverse-Transcription PCR (QRT-PCR) (Validation Set)

136 samples (histologically normal margins, OSCC and adjacent normal tissues) from an independent cohort of 30 patients were used for QRT-PCR validation. Patient clinical data for this validation set are summarized in Table 2. The maximum expression level of each gene in surgical margins was calculated, and these values were used to calculate the recurrence risk score for each patient. The risk scores were dichotomized using the median value of the training scores.

### RNA Isolation, Microarrays and Validation Experiments

### RNA Isolation

Total RNA was extracted from all tissues using Trizol reagent (Life Technologies, Inc., Burlington, ON, Canada), followed by purification using the Qiagen RNeasy kit/DNase RNase-free set (Qiagen, Valencia, Calif., USA), according to manufacturer's instructions. RNA was quantified by spectrophotometry and its quality was assessed using the 2100 Bioanalyzer (Firmware v.A.01.16, Agilent Technologies, Canada). All samples were of sufficient quantity and quality for arrays and quantitative real-time PCR (QRT-PCR) analyses.

### Oligonucleotide Array Experiments

The HG-U133A 2.0 plus oligonucleotide microarrays (Affymetrix, Santa Clara, Calif., USA) were used, which contain 40,000 probes representing 20,000 unique human genes. Labeling and hybridization to arrays were performed by The Centre for Applied Genomics, Medical and Related Sciences Centre (MaRS), Toronto, ON, Canada. Briefly, 10 μg of total RNA was used for cRNA amplification using the Invitrogen SuperScript kit (Life Technologies, Inc., Burlington, ON, Canada). Amplification and biotin labeling of antisense cRNA was performed using the Enzo® BioArray™ High Yield™ RNA transcript labeling kit (Enzo Diagnostics, Farmingdale, N.Y., USA), according to the manufacturer's instructions. Microarray slides were scanned using the GeneArray 2500 scanner (Agilent Technologies).

qRT-PCR Validation

qRT-PCR validation was performed using the 7900 Sequence Detection System and SYBR Green I fluorescent dye (Applied Biosystems, Foster City, Calif.) as previously described (31, 32). Primer sequences used are described in Table 3. Reactions were performed in duplicate for each sample and primer set. Dissociation curves were run for all reactions to ensure specificity. qRT-PCR data was normalized by the ΔΔCt method (33), with GAPDH as the internal control gene and a commercially available universal normal tongue RNA (Stratagene, Santa Clara, Calif.) as the reference sample.

TABLE 3
Primer sequences used for qPCR validation of the 4-gene signature

| Gene ID | Primer sequence | SEQ ID NO: |
|---|---|---|
| MMP1 | Forward: 5′-TGCTCATGCTTTTCAACCAG-3′ | SEQ ID NO: 1 |
| | Reverse: 5′-CCGCAACACGATGTAAGTTG-3′ | SEQ ID NO: 2 |
| COL4A1 | Forward: 5′-AGCAGAAGGACTGCCGGGGT-3′ | SEQ ID NO: 3 |
| | Reverse: 5′-CAATGCCTGGCTGGCCCACA-3′ | SEQ ID NO: 4 |
| THBS2 | Forward: 5′-GGTCGGCCTGCACTGTCACC-3′ | SEQ ID NO: 5 |
| | Reverse: 5′-GGGGAAGCTGCTGCACTGGG-3′ | SEQ ID NO: 6 |
| P4HA2 | Forward: 5′-AGGAGCTGCCAAAGCCCTGA-3′ | SEQ ID NO: 7 |
| | Reverse: 5′-ACCTGCTCCATCCACAACACCG-3′ | SEQ ID NO: 8 |
| GAPDH | Forward: 5′-GGCCTCCAAGGAGTAAGACC-3′ | SEQ ID NO: 9 |
| | Reverse: 5′-AGGGGTCTACATGGCAACTG-3′ | SEQ ID NO: 10 |

### Bioinformatics Analyses

All bioinformatic analyses of array data were performed in the R language and environment for statistical computing (version 2.10.0) implemented on CentOS 5.1 on an IBM HS21 Linux cluster (17).

Data Analysis of in-House Microarray Experiment

Microarray results from the in-house study were normalized by pre-processing using GCRMA normalization (39) with updated Entrez Gene-based chip definition files (10), using the affy R package (version 1.24.2) (41), along with microarray results for 14 normal oral tissue samples from healthy individuals (downloaded from GEO accession number GSE6791). Probesets with low expression (75th percentile below log 2(100)) or low variance (IQR on log 2 scale <0.25) were filtered (18), as well as the quality control probesets. The treat function from LIMMA: Linear Models for Microarray Analysis (version 3.2.1) (19) was used to identify genes ≧2-fold up-regulated in tumors compared to margins from the study, with FDR=0.01.

### Meta-Analysis of Published Datasets

A meta-analysis of five published and publicly available human array datasets was performed. Our prognostic signature was also based on deregulated genes identified through a meta-analysis of five published Affymetrix-based microarray studies (34-38). These studies were chosen since they profiled both oral carcinoma and normal oral cavity tissue and were publicly available. The goal was to generate a high-confidence list of up-regulated genes in oral squamous cell carcinoma (OSCC), with the hypothesis that up-regulation of this gene set in histologically normal margins leads to recurrence. Up-regulated genes were focused on only, since under-expression may not be accurately detectable in histologically normal margins that may contain only a fraction of genetically altered cells.

Each public data set was pre-processed using GCRMA normalization (39) with updated Entrez Gene-based chip definition files (10), using the affy R package (version 1.24.2) (41). Genes with evidence of tumor-normal differential expression across all datasets with a False Discovery Rate (FDR) of 0.01 and fold-change were identified using a rank product approach (42).

The intersection of genes identified in both the in-house microarray experiment and the meta-analysis was taken as the potential feature set for penalized Cox regression to generate a risk score for recurrence. Gene Ontology enrichment analysis was performed with the GOstats R package (version 2.12.0) (43). GOEAST (Go Enrichment Analysis Software Toolkit) (44) was used for graphical representation of GO annotations.

### Protein-Protein Interaction Network Analysis

Protein interaction network and pathway analyses were performed using the Interologous Interaction Database (I2D, v 1.71; http://ophid.utoronto.ca/i2d) (45). Network visualization and analysis was done in NAViGaTOR 2.1.15 (http://ophid.utoronto.ca/navigator) (46, 47). GO annotations and KEGG pathways of our data plus the literature data were identified using the Gene Annotation Co-Occurrence Discovery Tool (GeneCODIS) database (http://genecodis.dacya.ucm.es/) (48) and the Molecular Signatures Database (MSigDB) (http://www.broad.mit.edu/gsea/msigdb/index.jsp) (49).

### Penalized Cox Regression

The genes identified as up-regulated in tumors in both the meta-analysis and the in-house microarray experiment were used as the potential prognostic signature for recurrence. The maximum expression of these genes in the margins of each patient was calculated, and then converted to z-scores for each gene. LASSO penalized Cox regression was applied as implemented in the penalized R package (version 0.9-27) (20), using the maximum scaled expression value of each gene in any margin of a patient, to condition a linear risk score with local recurrence as the event of interest. The penalty parameter was selected by optimizing 10-fold cross-validated likelihood. The four genes with the largest coefficients were kept (MMP1, COL4A1, P4HA2 and THBS2), and the two genes with small coefficients were eliminated (PXDN and PMEPA1), which made a negligible contribution to the risk score.

### Effect of Reducing the Number of Available Margins

Taking advantage of having multiple margins for each patient, a bootstrap re-sampling simulation was used and a single margin from each patient was randomly selected, to calculate the value of the risk score for that patient. The risk scores for all patients were dichotomized at the median, and the hazard ratio between the high and low risk groups estimated by Cox regression. This process was repeated to simulate the distribution of hazard ratios when only one margin per patient is used to assess molecular risk of recurrence, in both the training and test patient cohorts. In the training set, the simulation using the mean z-transformed expression of all genes with FDR=0.01 was performed, as the risk score.

### Results

### Patient Characteristics: OSCC Recurrence

As shown in Tables 1 and 2, 8/23 patients (training set) and 7/30 patients (an independent validation set) had disease recurrence. Median time to local recurrence (by Kaplan Meier estimate) of patients in the training set was 33 months (range 2-34 months). Similarly, patients from the validation set recurred within 2-36 months. All patients had local recurrence, and some patients also had regional and/or distant failure; data are shown in Tables 1 and 2. Median (by reverse Kaplan-Meier estimate) and range of follow-up times of patients were 20 months (1.4-57 months) in the training set and 23 months (1-81 months) in the validation set.

TABLE 1
Clinicopathological data, recurrence and outcome data from 23 OSCC patients (N = 89 samples, training set)

| | | | Tobacco/ | | | | | | | |
|---|---|---|---|---|---|---|---|---|---|---|
| Case | Tumor Site | Age/Sex | Alcohol | TNM | Stage | Grade | REC* | TTREC | FU | Outcome |
| 1 | Tongue | 46/M | Y/Y | T4N2cM0 | IV | PD | Y⁺ | 24.7 | 24.7 | DOD |
| 2 | FOM | 83/F | Y/Y | T2N0M0 | II | MD | N | — | 16.7 | ANED |
| 3 | Buccal | 52/F | N/Y | T1N0M0 | I | MD | N | — | 15.8 | ANED |
| 4 | Tongue | 47/M | Y/Y | T2N0M0 | II | MD | N | — | 13.6 | ANED |
| 5 | Tongue | 46/M | Y/Y | T3N0M0 | III | PD | N | — | 18.7 | ANED |
| 6 | FOM | 64/F | Y/Y | T2N2cM0 | IV | MD | N | — | 11.5 | ANED |
| 7 | FOM | 48/M | Y/Y | T4N1M0 | IV | PD | N | — | 58.8 | ANED |
| 8 | Tongue | 47/F | N/N | T3N0M0 | II | MD | N | — | 53.2 | ANED |
| 9 | Alveolar | 74/F | N/N | T4N0M0 | IV | MD | N | — | 19.4 | ANED |
| 10 | Tongue | 44/M | N/N | T4N2cM0 | IV | MD | Y⁺ | 1.8 | 18.7 | DOD |
| 11 | Tongue | 74/F | N/N | T2N0M0 | IV | MD | N | — | 1.4 | ANED |
| 12 | Tongue | 73/M | Y/Y | T2N0M0 | II | MD | Y | 32 | 41 | AWD |
| 13 | Tongue | 71/F | Y/Y | T2N0M0 | II | MD | N | — | 23.9 | ANED |
| 14 | FOM | 71/M | Y/Y | T3N0M0 | III | MD | N | — | 13 | DOC |
| 15 | Alveolar | 58/M | Y/Y | T2N0M0 | II | MD | N | — | 54.6 | ANED |
| 16 | Tongue | 54/M | Y/Y | T2N0M0 | II | PD | N | — | 13 | DOC |
| 17 | Tongue | 37/M | N/N | T2N2bM0 | IV | MD | Y | 3.2 | 8 | DOD |
| 18 | Tongue | 59/M | Y/Y | T4N2cM0 | IV | MD | N | — | 57 | ANED |
| 19 | Tongue | 57/M | Y/Y | T4N2bM0 | IV | PD | Y⁺ | 2 | 4 | DOD |
| 20 | Tongue | 72/M | Y/Y | T2N1M0 | III | MD | N | — | 1.7 | ANED |
| 21 | Tongue | 60/M | N/N | T2N2bM0 | IV | MD | Y | 7.4 | 9.4 | DOD |
| 22 | Buccal | 78/F | Y/Y | T4N2bM0 | IV | PD | Y | 34 | 66 | AWD |
| 23 | Tongue | 52/F | N/N | T4N2bM0 | IV | MD | Y⁺ | 2.4 | 3.2 | DOD |

A tumor sample (OSCC) was collected from all patients
TNM: Tumor, Node, Metastasis. Pathological TNM is given
Grade: MD: moderately differentiated; PD: poorly differentiated
*REC: Recurrence. Y = Patients with local recurrence; ⁺Patients who also had regional and/or distant recurrence
TTREC: Time to recurrence (time between date of surgery and date of recurrence). Time is given in months.
FU: Follow-up (time between surgery and last follow-up, updated in March 2010). FU time is given in months
Outcome: ANED: patient is alive with no evidence of disease; AWD: alive with disease; DOD: died of disease; DOC: died of other causes

TABLE 2
Clinicopathological data, recurrence data and outcome data from 30 OSCC patients (N = 136 samples, validation set).

| | | | Tobacco/ | | | | | TTREC | FU | |
|---|---|---|---|---|---|---|---|---|---|---|
| Case | Tumor Site | Age/Sex | Alcohol | TNM | Stage | Grade | REC* | (months) | (months) | Outcome |
| 1 | FOM | 55/F | Y/N | T4N0M0 | IV | MD | N | — | 81 | ANED |
| 2 | FOM | 63/M | Y/Y | T3N0M0 | III | MD | N | — | 39 | ANED |
| 3 | Tongue | 75/M | Y/Y | T2N0M0 | II | MD | N | — | 21 | ANED |
| 4 | FOM | 74/M | Y/Y | T4N0M0 | IV | MD | N | — | 2 | DOC |
| 5 | Tongue | 74/M | Y/Y | T4N0M0 | IV | MD | N | — | 77 | ANED |
| 6 | Tongue | 61/F | Y/Y | T3N0M0 | III | PD | N | — | 59 | ANED |
| 7 | FOM | 48/M | Y/Y | T2N0M0 | II | PD | N | — | 3 | ANED |
| 8 | FOM | 85/F | Y/N | T2N0M0 | II | MD | Y | 36 | 48 | ANED |
| 9 | FOM | 74/M | Y/Y | T1N0M0 | I | MD | N | — | 52 | ANED |
| 10 | Retromolar | 55/M | Y/Y | T2N0M0 | II | MD | N | — | 12 | ANED |
| 11 | Tongue | 65/F | Y/N | T2N0M0 | II | MD | Y⁺ | 2 | 2 | DOD |
| 12 | Tongue | 71/M | Y/Y | T4N0M0 | IV | MD | N | — | 24 | ANED |
| 13 | Tongue | 51/M | Y/Y | T1N0M0 | I | MD | N | — | 46 | ANED |
| 14 | FOM | 76/M | Y/Y | T2N0M0 | II | MD | Y | 32 | 32 | AWD |
| 15 | Tongue | 60/F | Y/Y | T2N2cM0 | IV | PD | N | — | 52 | ANED |
| 16 | Tongue | 72/F | N/N | T3N0M0 | III | MD | N | — | 49 | ANED |
| 17 | Tongue + FOM | 50/M | Y/Y | T2N0M0 | II | MD | Y | 8 | 22 | ANED |
| 18 | Tongue + FOM | 53/M | Y/N | T3N1M0 | III | PD | N | — | 5 | ANED |
| 19 | Tongue | 81/M | N/N | T3N0M0 | III | MD | Y | 5 | 12 | ANED |
| 20 | Alveolar | 77/F | Y/Y | T4N2bM0 | IV | PD | Y | 19 | 20 | AWD |
| 21 | FOM | 52/M | Y/Y | T4N0M0 | IV | MD | N | — | 14 | ANED |
| 22 | Tongue | 51/M | Y/Y | T1N0M0 | I | MD | N | — | 22 | ANED |
| 23 | Tongue | 66/M | N/A | T4N0M0 | IV | MD | N | — | 16 | ANED |
| 24 | Tongue | 75/M | N/N | T1N0M0 | I | MD | N | — | 15 | ANED |
| 25 | Buccal mucosa | 68/M | Y**/Y | T2N0M0 | II | MD | N | — | 23 | ANED |
| 26 | Tongue | 50/F | Y/Y | T3N2aM0 | IV | MD | Y | 4 | 13 | DOD |
| 27 | Tongue + FOM | 59/M | Y/Y | T3N0M0 | III | MD | N | — | 21 | ANED |
| 28 | Tongue | 78/M | Y/Y | T2N0M0 | II | PD | N | — | 1 | AWD |
| 29 | Tongue | 68/F | Y/N | T3N1M0 | III | PD | N | — | 1 | AWD |
| 30 | Buccal mucosa | 70/M | N/Y | T4N2bM0 | IV | MD | N | — | 17 | ANED |

N/A: Information about tobacco and alcohol consumption was not available for Patient 23.
Y**: Patient 25 also chewed tobacco.
Patients 28 and 29 moved out of province, however the clinical follow-up (1 month after surgery) indicated the need for post-operative radiotherapy.
A tumor sample (OSCC) was collected from all patients
TNM: Tumor, Node, Metastasis. Pathological TNM is given
Grade: MD: moderately differentiated; PD: poorly differentiated
*REC: Recurrence. Y = patients had local recurrence; ⁺Patients who also had regional and/or distant recurrence
TTREC: Time to recurrence (time between date of surgery and date of recurrence). Time is given in months.
FU: Follow-up (time between surgery and last follow-up). FU time is given in months
Outcome: ANED: patient is alive with no evidence of disease; AWD: alive with disease; DOD: died of disease; DOC: died of other causes

### Differentially Expressed Genes in Margins, OSCC and Normal Oral Tissues

Meta-analysis of the five public data sets identified 667 up-regulated genes in OSCC compared to normal oral tissues from healthy individuals.

Data mining of both the meta-analysis of public datasets and the in-house microarray experiment, using the criteria of two-fold up-regulation in tumors with a FDR of 0.01, identified 138 up-regulated genes in OSCC (Table 4).

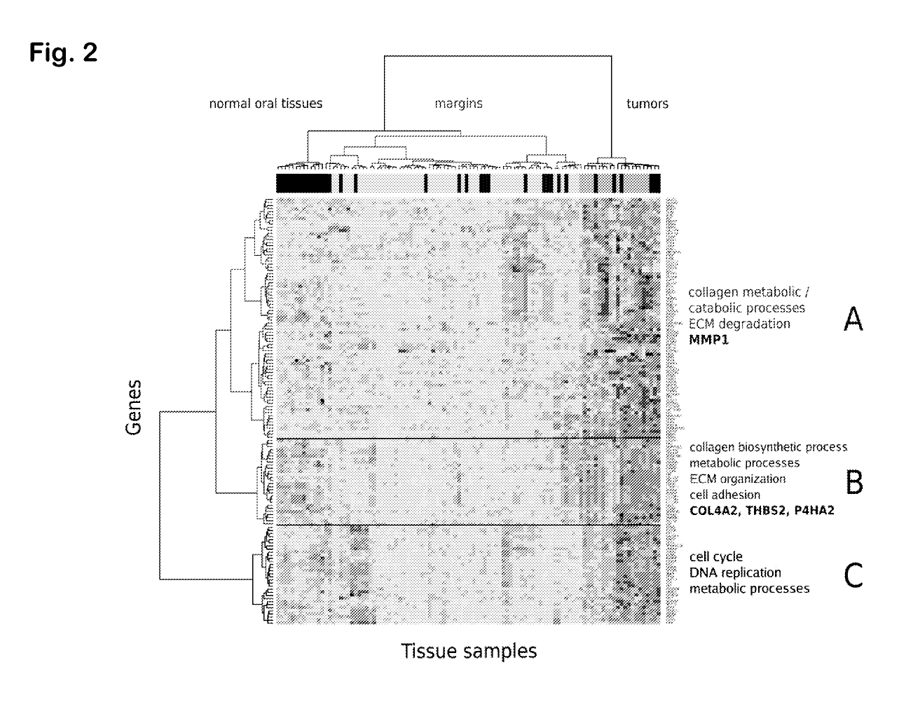

The expression patterns of these genes in tumors, margins, and normal oral tissue samples are shown as a heatmap in FIG. 2. All tumor and margin samples shown in the heatmap belong to the in-house microarray experiment. The normal oral tissue samples from healthy individuals were downloaded as raw CEL files from a public dataset (Gene Expression Omnibus (GEO) accession number GSE6791) and pre-processed with the in-house samples. These normal samples were used for comparison with margins and tumors only, but not used for gene selection, and to ensure that genes selected for validation were not altered in normal oral tissues from healthy individuals. As seen in the hierarchical clustering, the 138 genes accurately discriminate between the tumors, margins, and normal oral tissues (FIG. 2). Gene cluster “B”, in which three (COL4A1, P4HA2 and THBS2) of the four genes in the signature are found, shows frequent up-regulation in the surgical margins compared to the normal oral tissues. Strikingly, MMP1, found in gene cluster “A”, shows less frequent over-expression in the margins, but has extreme differential expression between margins and OSCCs (400-fold up-regulation in tumor compared to margins as detected by microarrays, and 800-fold up-regulation in tumor compared to margins, validated by QRT-PCR). The proteins encoded by these 138 genes are also shown in a protein interaction network that highlights the most highly inter-connected proteins (FIG. 1). In the heatmap, the main features of clusters A and B are the large number of interacting MMP proteins in cluster A, which contains MMP1, and collagens plus TGFB1 in cluster B, which also contains P4HA2, THBS2 and COL4A1 genes of the signature. The large number of MMPs and collagen proteins are closely connected; in particular, MMP9 interacts with both THBS2 and COL4A1, and indirectly with MMP1.

TABLE 4

| gene | raw | | Entrez | |
|---|---|---|---|---|
| symbol | p-value | FDR | Gene ID | Swissprot protein IDs |
| COL5A2 | 0.000160489 | 0.018036603 | 1290 | P05997, P78440, Q13908, Q53WR4, Q59GR4, Q6LDJ5, Q7KZ55, Q86XF6, |
| | | | | Q96QB0, Q96QB3 |
| THBS2 | 0.000391189 | 0.018036603 | 7058 | P35442 |
| SERPINH1 | 0.000394962 | 0.018036603 | 871 | P50454, P29043, Q5XPB4, Q6NSJ6, Q8IY96, Q9NP88 |
| MMP1 | 0.000566879 | 0.019415611 | 4312 | P03956, P08156 |
| COL5A1 | 0.001330918 | 0.036467147 | 1289 | P20908, Q15094, Q5SUX4 |
| CTHRC1 | 0.002253115 | 0.041633991 | 115908 | Q96CG8, Q6UW91, Q8IX63 |
| COL4A1 | 0.002406603 | 0.041633991 | 1282 | P02462, A7E2W4, B1AM70, Q1P9S9, Q5VWF6, Q86X41, Q8NF88, Q9NYC5 |
| PXDN | 0.002431182 | 0.041633991 | 7837 | Q92626, A8QM65, Q4KMG2 |
| COL3A1 | 0.002909613 | 0.044290771 | 1281 | P02461, P78429, Q15112, Q16403, Q53S91, Q541P8, Q6LDB3, Q6LDJ2, |
| | | | | Q6LDJ3, Q7KZ56, Q8N6U4 |
| SERPINE2 | 0.006445808 | 0.088307564 | 5270 | P07093 |
| PLOD2 | 0.007654179 | 0.089127764 | 5352 | O00469, Q8N170 |
| POSTN | 0.007806811 | 0.089127764 | 10631 | Q15063, Q15064, Q5VSY5, Q8IZF9 |
| COL4A2 | 0.008739083 | 0.092096486 | 1284 | P08572, Q14052, Q548C3, Q5VZA9, Q66K23 |
| COL1A2 | 0.012556003 | 0.122869459 | 1278 | P08123, P02464, Q13897, Q13997, Q13998, Q14038, Q14057, Q15177, |
| | | | | Q15947, Q16480, Q16511, Q7Z5S6, Q9UEB6, Q9UEF9, Q9UM83, Q9UMI1, |
| | | | | Q9UML5, Q9UMM6, Q9UPH0 |
| COL1A1 | 0.016500957 | 0.150708744 | 1277 | P02452, O76045, P78441, Q13896, Q13902, Q13903, Q14037, Q14992, |
| | | | | Q15176, Q15201, Q16050, Q59F64, Q7KZ30, Q7KZ34, Q8IVI5, Q8N473, |
| | | | | Q9UML6, Q9UMM7 |
| P4HA2 | 0.020132479 | 0.172384352 | 8974 | O15460, Q8WWN0 |
| PDPN | 0.023193407 | 0.18691157 | 10630 | Q86YL7, O60836, O95128, Q7L375, Q8NBQ8, Q8NBR3 |
| TNC | 0.027289968 | 0.205014011 | 3371 | P24821, Q14583, Q15567 |
| SERPINE1 | 0.028663862 | 0.205014011 | 5054 | P05121 |
| MFAP2 | 0.029929053 | 0.205014011 | 4237 | P55001 |
| MMP10 | 0.03386357 | 0.220919479 | 4319 | P09238 |
| TLR2 | 0.035992033 | 0.224132204 | 7097 | O60603, O15454, Q8NI00 |
| C4orf48 | 0.040998643 | 0.24420931 | 401115 | NA |
| PMEPA1 | 0.043047853 | 0.245731494 | 56937 | Q969W9, Q5TDR6, Q96B72, Q9UJD3 |
| GREM1 | 0.044941241 | 0.246277998 | 26585 | O60565, Q52LV3, Q8N914, Q8N936 |
| C9orf30 | 0.047491771 | 0.250245099 | 91283 | Q96H12, Q5T726, Q5T727, Q5T728 |
| FAP | 0.054314507 | 0.27469036 | 2191 | Q12884, O00199, Q86Z29, Q99998, Q9UID4 |
| EGFL6 | 0.056141096 | 0.27469036 | 25975 | Q8IUX8, Q6UXJ1, Q8NBV0, Q8WYG3, Q9NY67, Q9NZL7, Q9UFK6 |
| LPCAT1 | 0.073595411 | 0.347674874 | 79888 | Q8NF37, Q1HAQ1, Q7Z4G6, Q8N3U7, Q8WUL8, Q9GZW6 |
| FADD | 0.089685837 | 0.399237022 | 8772 | Q13158, Q14866 |
| CALU | 0.091678397 | 0.399237022 | 813 | O43852, O60456, Q6FHB9, Q96RL3, Q9NR43 |
| MMP3 | 0.09496235 | 0.399237022 | 4314 | P08254, Q3B7S0, Q6GRF8 |
| CHST2 | 0.099307497 | 0.399237022 | 9435 | Q9Y4C5, Q2M370, Q9GZN5, Q9UED5, Q9Y6F2 |
| ASPRV1 | 0.099521617 | 0.399237022 | 151516 | Q53RT3, Q8N5P2, Q96LT3, Q96N43 |
| NEFL | 0.10199486 | 0.399237022 | 4747 | P07196, Q16154, Q8IU72 |
| ATAD2 | 0.118751281 | 0.451914597 | 29028 | Q6PL18, Q14CR1, Q658P2, Q68CQ0, Q6PJV6, Q8N890, Q9UHS5 |
| OAS3 | 0.128879449 | 0.471482467 | 4940 | Q9Y6K5, Q9H3P5 |
| RAB31 | 0.130776159 | 0.471482467 | 11031 | Q13636, Q15770, Q9HC00 |
| XAF1 | 0.138700984 | 0.475771288 | 54739 | Q6GPH4, A2T931, A2T932, A8K2L1, A8K9Y3, Q6MZE8, Q8N557, Q99982 |
| CDC20 | 0.138911325 | 0.475771288 | 991 | Q12834, Q5JUY4, Q9BW56, Q9UQI9 |
| CXCL13 | 0.171952844 | 0.574574138 | 10563 | O43927 |
| DDX60 | 0.179308438 | 0.578283203 | 55601 | Q8IY21, Q6PK35, Q9NVE3 |
| MELK | 0.185160244 | 0.578283203 | 9833 | Q14680, Q7L3C3 |
| TK1 | 0.185725992 | 0.578283203 | 7083 | P04183, Q969V0, Q9UMG9 |
| TRIP13 | 0.196658079 | 0.579447478 | 9319 | Q15645, O15324 |
| CEP55 | 0.20005281 | 0.579447478 | 55165 | Q53EZ4, Q32WF5, Q3MV20, Q5VY28, Q6N034, Q96H32, Q9NVS7 |
| ANLN | 0.202107101 | 0.579447478 | 54443 | Q9NQW6, Q5CZ78, Q6NSK5, Q9H8Y4, Q9NVN9, Q9NVP0 |
| TNFRSF12A | 0.203997325 | 0.579447478 | 51330 | Q9NP84, Q9HCS0 |
| CXCL11 | 0.211055586 | 0.579447478 | 6373 | O14625, Q53YA3, Q92840 |
| FAT1 | 0.221372288 | 0.579447478 | 2195 | NA |
| ECT2 | 0.222773156 | 0.579447478 | 1894 | Q9H8V3, Q9NSV8, Q9NVW9 |
| IFIT3 | 0.226828446 | 0.579447478 | 3437 | O14879, Q99634, Q9BSK7 |
| APOL1 | 0.228051298 | 0.579447478 | 8542 | O14791, O60804, Q5R3P7, Q5R3P8, Q96AB8, Q96PM4, Q9BQ03 |
| TOP2A | 0.228395356 | 0.579447478 | 7153 | P11388, Q71UN1, Q71UQ5, Q9HB24, Q9HB25, Q9HB26, Q9UP44, Q9UQP9 |
| SULF1 | 0.236960355 | 0.590246703 | 23213 | Q8IWU6, Q86YV8, Q8NCA2, Q9UPS5 |
| GINS2 | 0.24637922 | 0.599561688 | 51659 | Q9Y248, Q6IAG9 |
| RTP4 | 0.254669839 | 0.599561688 | 64108 | Q96DX8, Q9H4F3 |
| MCM2 | 0.254881869 | 0.599561688 | 4171 | P49736, Q14577, Q15023, Q8N2V1, Q969W7, Q96AE1, Q9BRM7 |
| DTL | 0.258205399 | 0.599561688 | 51514 | Q9NZJ0, Q5VT77, Q96SN0, Q9NW03, Q9NW34, Q9NWM5 |
| TPX2 | 0.278168327 | 0.635151012 | 22974 | Q9ULW0, Q9H1R4, Q9NRA3, Q9UFN9, Q9UL00, Q9Y2M1 |
| KIF14 | 0.287300477 | 0.636219991 | 9928 | Q15058, Q14CI8, Q4G0A5, Q5T1W3 |
| ODZ2 | 0.287924376 | 0.636219991 | 57451 | Q9NT68, Q9ULU2 |
| TYMS | 0.294980683 | 0.64146593 | 7298 | P04818 |
| CDKN3 | 0.302250822 | 0.647005665 | 1033 | Q16667, Q99585, Q9BPW7, Q9BY36, Q9C042, Q9C047, Q9C049, Q9C051, Q9C053 |
| NUP155 | 0.315858996 | 0.656717536 | 9631 | O75694, Q9UBE9, Q9UFL5 |
| IFI44 | 0.319369156 | 0.656717536 | 10561 | Q8TCB0 |
| AURKA | 0.335933252 | 0.656717536 | 6790 | O14965, O60445, O75873, Q9BQD6, Q9UPG5 |
| SOAT1 | 0.336530182 | 0.656717536 | 6646 | P35610, A6NC40, A9Z1V7, Q5T0X4, Q8N1E4 |
| BST2 | 0.339547397 | 0.656717536 | 684 | Q10589, Q53G07 |
| SLC3A2 | 0.343107251 | 0.656717536 | 6520 | P08195, Q13543 |
| XPR1 | 0.344757168 | 0.656717536 | 9213 | Q9UBH6, O95719, Q7L8K9, Q8IW20, Q9NT19, Q9UFB9 |
| RSAD2 | 0.345136223 | 0.656717536 | 91543 | Q8WXG1, Q8WVI4 |
| PARP12 | 0.357258863 | 0.670472113 | 64761 | Q9HOJ9, Q9H610, Q9NP36, Q9NTI3 |
| RFC4 | 0.379577747 | 0.697665261 | 5984 | P35249, Q6FHX7 |
| SPP1 | 0.385960585 | 0.697665261 | 6696 | P10451, Q15681, Q15682, Q15683, Q8NBK2, Q96IZ1 |
| LAPTM4B | 0.387025984 | 0.697665261 | 55353 | Q86VI4, Q3ZCV5, Q7L909, Q86VH8, Q9H060 |
| DDX58 | 0.395929599 | 0.700145832 | 23586 | O95786, Q5HYE1, Q5VYT1, Q9NT04 |
| TPBG | 0.398623175 | 0.700145832 | 7162 | Q13641 |
| C12orf75 | 0.433665171 | 0.752052259 | 387882 | NA |
| IFI27 | 0.44734861 | 0.758932416 | 3429 | P40305, Q53YA6, Q6IEC1, Q96BK3 |
| MYO1B | 0.449793944 | 0.758932416 | 4430 | O43795, O43794, Q7Z6L5 |
| CXCL9 | 0.463353298 | 0.758932416 | 4283 | Q07325, Q503B4 |
| SHCBP1 | 0.467925099 | 0.758932416 | 79801 | Q8NEM2, Q96N60, Q9BVS0, Q9H6P6 |
| KRT17 | 0.472009884 | 0.758932416 | 3872 | Q04695, A5Z1M9, A5Z1N0, A5Z1N1, A5Z1N2, A6NDV6, A6NKQ2, Q6IP98, Q8N1P6 |
| PPP1R14C | 0.474360549 | 0.758932416 | 81706 | Q8TAE6, Q5VY83, Q96BB1, Q9H277 |
| KRT16 | 0.47641013 | 0.758932416 | 3868 | P08779, P30654, Q16402, Q9UBG8 |
| DFNA5 | 0.505508601 | 0.786816067 | 1687 | O60443, O14590, Q08AQ8, Q9UBV3 |
| IFI35 | 0.509276502 | 0.786816067 | 3430 | P80217, Q92984, Q99537, Q9BV98 |
| SESN3 | 0.511143284 | 0.786816067 | 143686 | P58005, Q96AD1 |
| ITGA6 | 0.520391697 | 0.791501482 | 3655 | P23229, Q08443, Q14646, Q16508, Q9UN03 |
| CMPK2 | 0.52574186 | 0.791501482 | 129607 | Q5EBM0, A2RUB0, A5D8T2, Q6ZRU2, Q96AL8 |
| AGTRAP | 0.551301366 | 0.800500595 | 57085 | Q6RW13, Q5SNV4, Q5SNV5, Q96AC0, Q96PL4, Q9NRW9 |
| APOBEC3B | 0.5519403 | 0.800500595 | 9582 | Q9UH17, O95618, Q5IFJ4, Q7Z2N3, Q7Z6D6, Q9UE74 |
| MED10 | 0.554451759 | 0.800500595 | 84246 | Q9BTT4 |
| PLEK2 | 0.555091653 | 0.800500595 | 26499 | Q9NYT0, Q96JT0 |
| FBXO45 | 0.567367642 | 0.809680906 | 200933 | P0C2W1 |
| TGFBI | 0.578448545 | 0.816984027 | 7045 | Q15582, O14471, O14472, O14476, O43216, O43217, O43218, O43219 |
| TFRC | 0.586296137 | 0.819618069 | 7037 | P02786, Q59G55, Q9UCN0, Q9UCU5, Q9UDF9, Q9UK21 |
| PTGFRN | 0.594449831 | 0.822622493 | 5738 | Q9P2B2, Q8N2K6 |
| OCIAD2 | 0.635855176 | 0.861568359 | 132299 | Q56VL3, Q8N544 |
| KYNU | 0.637279837 | 0.861568359 | 8942 | Q16719 |
| IFI30 | 0.641459654 | 0.861568359 | 10437 | P13284, Q9UL08 |
| ISG15 | 0.659050167 | 0.873448209 | 9636 | P05161, Q7Z2G2, Q96GF0 |
| TYMP | 0.663055575 | 0.873448209 | 1890 | P19971, A8MW15, Q13390, Q8WVB7 |
| UBE2L6 | 0.718067972 | 0.936907735 | 9246 | O14933, Q9UEZ0 |
| TMEM206 | 0.745560913 | 0.948592572 | 55248 | Q9H813, O6IA87, Q9NV85 |
| MICB | 0.747051702 | 0.948592572 | 4277 | Q29980, A6NP85, B0UZ10, O14499, O14500, O19798, O19799, O19800, O19801, |
| | | | | O19802, O19803, O78099, O78100, O78101, O78102, O78103, O78104, P79525, |
| | | | | P79541, Q5GR31, Q5GR37, Q5GR41, Q5GR42, Q5GR43, Q5GR44, Q5GR46, Q5GR48, |
| | | | | Q5RIY6, Q5SSK1, Q5ST25, Q7JK51, Q7YQ89, Q9MY18, Q9MY19, Q9MY20, Q9UBH4, |
| | | | | Q9UBZ8, Q9UEJ0 |
| MMP7 | 0.772485889 | 0.948592572 | 4316 | P09237, Q9BTK9 |
| SEMA3C | 0.775596803 | 0.948592572 | 10512 | Q99985 |
| PSMB2 | 0.776872194 | 0.948592572 | 5690 | P49721, P31145, Q9BWZ9 |
| EPSTI1 | 0.778930626 | 0.948592572 | 94240 | Q96J88, Q8IVC7, Q8NDQ7 |
| LAMB3 | 0.786100871 | 0.948592572 | 3914 | Q13751, O14947, Q14733, Q9UJK4, Q9UJL1 |
| ITGA3 | 0.790493916 | 0.948592572 | 3675 | P26006 |
| FST | 0.800183609 | 0.948592572 | 10468 | P19883, Q9BTH0 |
| SNAI2 | 0.801961098 | 0.948592572 | 6591 | O43623 |
| OAS1 | 0.803252298 | 0.948592572 | 4938 | P00973, P04820, P29080, P29081, P78485, P78486, Q16700, Q16701, Q1PG42, |
| | | | | Q53GC5, Q53YA4, Q6A1Z3, Q6IPC6, Q6P7N9, Q96J61 |
| BID | 0.810111905 | 0.948592572 | 637 | P55957, Q549M7, Q71T04, Q7Z4M9, Q8IY86 |
| IDO1 | 0.836882791 | 0.950552539 | 3620 | NA |
| LAMP3 | 0.844047285 | 0.950552539 | 27074 | Q9UQV4, O94781, Q8NEC8 |
| MMP12 | 0.851127788 | 0.950552539 | 4321 | P39900, Q2M1L9 |
| WDR54 | 0.852167913 | 0.950552539 | 84058 | Q9H977, Q53H85, Q86V45 |
| AIM2 | 0.855313208 | 0.950552539 | 9447 | O14862, A8K7M7, Q5T3V9, Q96FG9 |
| RBP1 | 0.858046064 | 0.950552539 | 5947 | P09455 |
| BNC1 | 0.860354123 | 0.950552539 | 646 | Q01954, Q15840 |
| CA2 | 0.872414806 | 0.956166627 | 760 | P00918, Q6FI12, Q96ET9 |
| CDH3 | 0.890534814 | 0.968279917 | 1001 | P22223, Q05DI6 |
| RUVBL1 | 0.908451873 | 0.972869866 | 8607 | Q9Y265, P82276, Q1KMR0, Q53HK5, Q53HL7, Q53Y27, Q9BSX9 |
| WARS | 0.912675717 | 0.972869866 | 7453 | P23381, P78535, Q9UDL3 |
| SLC16A1 | 0.916059947 | 0.972869866 | 6566 | P53985, Q9NSJ9 |
| CDC25B | 0.930601451 | 0.97725477 | 994 | P30305, O43551, Q13971, Q5JX77, Q6RSS1, Q9BRA6 |
| NETO2 | 0.938777691 | 0.97725477 | 81831 | Q8NC67, Q7Z381, Q8ND51, Q96SP4, Q9NVY8 |
| IFI6 | 0.941588538 | 0.97725477 | 2537 | P09912, Q13141, Q13142, Q969M8 |
| MMP9 | 0.950108406 | 0.978683095 | 4318 | P14780, Q3LR70, Q8N725, Q9H4Z1 |
| IRF6 | 0.972588079 | 0.99080786 | 3664 | O14896 |
| KIF20A | 0.982729113 | 0.99080786 | 10112 | O95235 |
| GALNT6 | 0.983575685 | 0.99080786 | 11226 | Q8NCL4, Q8IYH4, Q9H6G2, Q9UIV5 |
| FERMT1 | 0.998228636 | 0.998228636 | 55612 | Q9BQL6, Q8IX34, Q8IYH2, Q9NWM2, Q9NXQ3 |

Results of Gene Ontology (GO) Enrichment Analysis of all 138 Genes are Presented in Table 5.

TABLE 5
(clade 1) Gene to GO BP test for over-representation

| GOBPID | Pvalue | OddsRatio | ExpCount | Count | Size | Term |
|---|---|---|---|---|---|---|
| GO: 0048015 | 0.000300000 | 26.2713 | 0 | 3 | 58 | phosphoinositide-mediated |
| | | | | | | signaling |
| GO: 0006260 | 0.000000000 | 16.6448 | 0 | 6 | 201 | DNA replication |
| GO: 0000280 | 0.000100000 | 12.0131 | 1 | 5 | 220 | nuclear division |
| GO: 0007067 | 0.000100000 | 12.0131 | 1 | 5 | 220 | mitosis |
| GO: 0000087 | 0.000100000 | 11.9004 | 1 | 5 | 222 | M phase of mitotic cell cycle |
| GO: 0048285 | 0.000200000 | 11.6275 | 1 | 5 | 227 | organelle fission |
| GO: 0000279 | 0.000600000 | 8.4041 | 1 | 5 | 310 | M phase |
| GO: 0006259 | 0.000700000 | 6.6578 | 1 | 6 | 482 | DNA metabolic process |

Source for annotations: http://cbio.mskcc.org/CancerGenes/

| | | | | Refseq | | | | |
|---|---|---|---|---|---|---|---|---|
| | | | | Protein (linked | | UCSC | | |
| Entrez | Gene | | Refseq | to MSKCC | | Genomic | | |
| ID | Symbol | Gene Name | mRNA | Mapback) | Ensembl Gene ID | Coordinates | GO Categories | Sources |
| 54443 | ANLN | anillin, actin | NM_018685 | NP_061155.2 | ENSG00000011426 | chr7: 36396160-36458734 | actin binding; cell cycle; contractile | |
| | | binding protein | | | | | ring; cytokinesis; mitosis; nucleus; regulation of exit | |
| | | | | | | | from mitosis; septin ring assembly | |
| 9582 | APOBEC3B | apolipoprotein B | NM_004900 | NP_004891.3 | ENSG00000179750 | chr22: 37708404-37718396 | hydrolase activity; hydrolase activity, acting on | |
| | | mRNA editing | | | | | carbon-nitrogen (but not peptide) bonds, in cyclic | |
| | | enzyme, | | | | | amidines; zinc ion binding | |
| | | catalytic | | | | | | |
| | | polypeptide-like | | | | | | |
| | | 3B | | | | | | |
| 29028 | ATAD2 | ATPase family, | NM_014109 | NP_054828.2 | ENSG00000156802 | chr8: 124402554-124477778 | ATP binding; nucleoside-triphosphatase | |
| | | AAA domain | | | | | activity; nucleotide binding | |
| | | containing 2 | | | | | | |
| 6790 | AURKA | aurora kinase A | NM_003600 | NP_940839.1 | ENSG00000087586 | chr20: 54378620-54396660 | ATP binding; kinase activity; mitosis; mitotic cell | |
| | | | | | | | cycle; nucleotide binding; nucleus; phosphoinositide- | |
| | | | | | | | mediated signaling; protein amino acid | |
| | | | | | | | phosphorylation; protein binding; protein kinase | |
| | | | | | | | activity; protein serine/threonine kinase | |
| | | | | | | | activity; regulation of protein stability; spindle; spindle | |
| | | | | | | | organization and biogenesis; transferase | |
| | | | | | | | activity; ubiquitin protein ligase binding | |
| 646 | BNC1 | basonuclin 1 | NM_001717 | NP_001708.3 | ENSG00000169594 | chr15: 81717197-81744384 | epidermis development; intracellular; metal ion | |
| | | | | | | | binding; nucleic acid binding; nucleus; positive | |
| | | | | | | | regulation of cell proliferation; regulation of | |
| | | | | | | | transcription, DNA- | |
| | | | | | | | dependent; transcription; transcription factor | |
| | | | | | | | activity; zinc ion binding | |
| 991 | CDC20 | cell division | NM_001255 | NP_001246.2 | ENSG00000117399 | chr1: 43597473-43601387 | cell cycle; cell division; mitosis; protein | |
| | | cycle 20 | | | | | binding; regulation of progression through cell | |
| | | homolog | | | | | cycle; spindle; ubiquitin cycle; ubiquitin-dependent | |
| | | (S. cerevisiae) | | | | | protein catabolic process | |
| 1001 | CDH3 | cadherin 3, type | NM_001793 | NP_001784.2 | ENSG00000062038 | chr16: 67236783-67289804 | calcium ion binding; homophilic cell adhesion; integral | |
| | | 1, P-cadherin | | | | | to membrane; plasma membrane; protein | |
| | | (placental) | | | | | binding; response to stimulus; visual perception | |
| 55165 | CEP55 | centrosomal | NM_018131, | NP_060601.3, | ENSG00000138180 | chr10: 95249798-95277900 | cell cycle; cell division; mitosis | |
| | | protein 55 kDa | NM_001127182 | NP_001120654.1 | | | | |
| 51514 | DTL | denticleless | NM_016448 | NP_057532.2 | ENSG00000143476 | chr1: 210275855-210342905 | DNA replication; nucleus; protein binding; response to | |
| | | homolog | | | | | DNA damage stimulus; ubiquitin cycle | |
| | | (Drosophila) | | | | | | |
| 1894 | ECT2 | epithelial cell | NM_018098 | NP_060568.3 | ENSG00000114346 | chr3: 173955014-174020721 | guanyl-nucleotide exchange factor | Oncogene |
| | | transforming | | | | | activity; intracellular; intracellular signaling | |
| | | sequence 2 | | | | | cascade; positive regulation of I-kappaB kinase/NF- | |
| | | oncogene | | | | | kappaB cascade; protein binding; regulation of Rho | |
| | | | | | | | protein signal transduction; Rho guanyl-nucleotide | |
| | | | | | | | exchange factor activity; signal transducer activity | |
| 2195 | FAT1 | FAT tumor | NM_005245 | NP_005236.2 | ENSG00000083857 | chr4: 187746739-187867975 | anatomical structure morphogenesis; calcium ion | Tumor |
| | | suppressor | | | | | binding; cell adhesion; cell-cell signaling; homophilic | Suppressor |
| | | homolog 1 | | | | | cell adhesion; integral to plasma | |
| | | (Drosophila) | | | | | membrane; membrane; protein binding | |
| 55612 | FERMT1 | chromosome 20 | NM_017671 | NP_060141.3 | ENSG00000101311 | chr20: 6005819-6048201 | | |
| | | open reading | | | | | | |
| | | frame 42 | | | | | | |
| 51659 | GINS2 | GINS complex | NM_016095 | NP_057179.1 | ENSG00000131153 | chr16: 84269318-84280005 | DNA replication; nucleus | |
| | | subunit 2 (Psf2 | | | | | | |
| | | homolog) | | | | | | |
| 3664 | IRF6 | interferon | NM_006147 | NP_006138.1 | ENSG00000117595 | chr1: 208028387-208041381 | intracellular; nucleus; regulation of transcription, DNA- | |
| | | regulatory factor 6 | | | | | dependent; transcription; transcription factor activity | |
| 9928 | KIF14 | kinesin family | NM_014875 | NP_055690.1 | ENSG00000118193 | chr1: 198789138-198854474 | ATP binding; microtubule; microtubule associated | |
| | | member 14 | | | | | complex; microtubule motor activity; microtubule- | |
| | | | | | | | based movement; nucleotide binding | |
| 10112 | KIF20A | kinesin family | NM_005733 | NP_005724.1 | ENSG00000112984 | chr5: 137543268-137551001 | ATP binding; Golgi | |
| | | member 20A | | | | | apparatus; microtubule; microtubule associated | |
| | | | | | | | complex; microtubule motor activity; microtubule- | |
| | | | | | | | based movement; nucleotide binding; protein | |
| | | | | | | | transport; transporter activity; vesicle-mediated | |
| | | | | | | | transport | |
| 3914 | LAMB3 | laminin, beta 3 | NM_001017402 | NP_001121113.1 | ENSG00000196878 | chr1: 207855238-207890912 | basement membrane; cell adhesion; electron carrier | |
| | | | | | | | activity; electron transport; epidermis | |
| | | | | | | | development; heme binding; iron ion binding; laminin-5 | |
| | | | | | | | complex; protein binding; proteinaceous extracellular | |
| | | | | | | | matrix; structural molecule activity | |
| 27074 | LAMP3 | lysosomal- | NM_014398 | NP_055213.2 | ENSG00000078081 | chr3: 184324562-184363137 | cell proliferation; integral to membrane; lysosomal | |
| | | associated | | | | | membrane; membrane | |
| | | membrane | | | | | | |
| | | protein 3 | | | | | | |
| 4171 | MCM2 | minichromosome | NM_004526 | NP_004517.2 | ENSG00000073111 | chr3: 128799999-128823306 | ATP binding; cell cycle; chromatin; DNA binding; DNA | |
| | | maintenance | | | | | replication; DNA replication initiation; DNA replication | |
| | | complex | | | | | origin binding; DNA unwinding during | |
| | | component 2 | | | | | replication; DNA-dependent ATPase activity; metal | |
| | | | | | | | ion binding; nuclear origin of replication recognition | |
| | | | | | | | complex; nucleosome assembly; nucleotide | |
| | | | | | | | binding; nucleus; protein binding; regulation of | |
| | | | | | | | transcription, DNA-dependent; transcription; zinc ion | |
| | | | | | | | binding | |
| 9833 | MELK | maternal | NM_014791 | NP_055606.1 | ENSG00000165304 | chr9: 36571678-36667334 | ATP binding; nucleotide binding; protein amino acid | |
| | | embryonic | | | | | phosphorylation; protein serine/threonine kinase | |
| | | leucine zipper | | | | | activity; transferase activity | |
| | | kinase | | | | | | |
| 81831 | NETO2 | neuropilin (NRP) | NM_018092 | NP_060562.3 | ENSG00000171208 | chr16: 45674632-45735024 | integral to membrane; membrane; receptor activity | |
| | | and tolloid (TLL)- | | | | | | |
| | | like 2 | | | | | | |
| 9631 | NUP155 | nucleoporin | NM_004298, | NP_004289.1, | ENSG00000113569 | chr5: 37327758-37406836 | nuclear pore; nucleocytoplasmic | |
| | | 155 kDa | NM_153485 | NP_705618.1 | | | transport; nucleocytoplasmic transporter | |
| | | | | | | | activity; nucleus; structural constituent of nuclear | |
| | | | | | | | pore; transport; transporter activity | |
| 132299 | OCIAD2 | OCIA domain | NM_001014446, | NP_001014446.1, | ENSG00000145247 | chr4: 48582257-48601323 | | |
| | | containing 2 | NM_152398 | NP_689611.1 | | | | |
| 26499 | PLEK2 | pleckstrin 2 | NM_016445 | NP_057529.1 | ENSG00000100558 | chr14: 66923798-66948529 | actin cytoskeleton organization and | |
| | | | | | | | biogenesis; cytoskeleton; intracellular signaling | |
| | | | | | | | cascade; membrane | |
| 5738 | PTGFRN | prostaglandin F2 | NM_020440 | NP_065173.2 | ENSG00000134247 | chr1: 117254348-117331112 | endoplasmic reticulum; integral to | |
| | | receptor | | | | | membrane; membrane; negative regulation of protein | |
| | | negative | | | | | biosynthetic process; protein binding | |
| | | regulator | | | | | | |
| 79801 | SHCBP1 | SHC SH2- | NM_024745 | NP_079021.3 | ENSG00000171241 | chr16: 45173141-45212772 | protein binding; SH2 domain binding | |
| | | domain binding | | | | | | |
| | | protein 1 | | | | | | |
| 7083 | TK1 | thymidine kinase | NM_003258 | NP_003249.3 | ENSG00000167900 | chr17: 73682434-73694670 | ATP binding; cytoplasm; DNA replication; kinase | |
| | | 1, soluble | | | | | activity; nucleobase, nucleoside, nucleotide and | |
| | | | | | | | nucleic acid metabolic process; nucleotide | |
| | | | | | | | binding; thymidine kinase activity; transferase activity | |
| 7153 | TOP2A | topoisomerase | NM_001067 | NP_001058.2 | http://www.ensembl.org/ | chr17: 35799296-35827569 | apoptotic chromosome condensation; ATP | |
| | | (DNA) II alpha | | | Homo_sapiens/Search/ | | binding; centriole; chromatin | |
| | | 170 kDa | | | Summary?species= | | binding; chromosome; chromosome segregation; DNA | |
| | | | | | Homo_sapiens;idx=;q= | | ligation; DNA repair; DNA replication; DNA | |
| | | | | | | | topoisomerase (ATP-hydrolyzing) activity; DNA | |
| | | | | | | | topoisomerase complex (ATP-hydrolyzing); DNA | |
| | | | | | | | topological change; DNA-dependent ATPase | |
| | | | | | | | activity; drug binding; histone deacetylase | |
| | | | | | | | binding; nucleolus; nucleoplasm; nucleotide | |
| | | | | | | | binding; nucleus; phosphoinositide-mediated | |
| | | | | | | | signaling; positive regulation of apoptosis; positive | |
| | | | | | | | regulation of retroviral genome replication; protein C- | |
| 22974 | TPX2 | TPX2, | NM_012112 | NP_036244.2 | ENSG00000088325 | chr20: 29808940-29852544 | ATP binding; cell proliferation; GTP | |
| | | microtubule- | | | | | binding; mitosis; nucleus; protein binding; spindle pole | |
| | | associated, | | | | | | |
| | | homolog | | | | | | |
| | | (Xenopus laevis) | | | | | | |
| 9319 | TRIP13 | thyroid hormone | NM_004237 | NP_004228.1 | ENSG00000071539 | chr5: 946113-970218 | ATP binding; nucleoside-triphosphatase | |
| | | receptor | | | | | activity; nucleotide binding; nucleus; transcription | |
| | | interactor 13 | | | | | cofactor activity; transcription from RNA polymerase | |
| | | | | | | | II promoter | |
| 7298 | TYMS | thymidylate | NM_001071 | NP_001062.1 | ENSG00000176890 | chr18: 647742-662997 | deoxyribonucleoside monophosphate biosynthetic | Oncogene, |
| | | synthetase | | | | | process; DNA repair; DNA replication; dTMP | Stability |
| | | | | | | | biosynthetic process; methyltransferase | |
| | | | | | | | activity; nucleobase, nucleoside, nucleotide and | |
| | | | | | | | nucleic acid metabolic process; nucleotide | |
| | | | | | | | biosynthetic process; phosphoinositide-mediated | |
| | | | | | | | signaling; thymidylate synthase activity; transferase | |
| | | | | | | | activity | |
| 9213 | XPR1 | xenotropic and | NM_004736 | NP_004727.2 | ENSG00000143324 | chr1: 178867960-179119825 | G-protein coupled receptor activity; G-protein coupled | |
| | | polytropic | | | | | receptor protein signaling pathway; integral to | |
| | | retrovirus | | | | | membrane; integral to plasma membrane; plasma | |
| | | receptor | | | | | membrane; receptor activity | |

### Four-Gene Signature Predictive of OSCC Recurrence

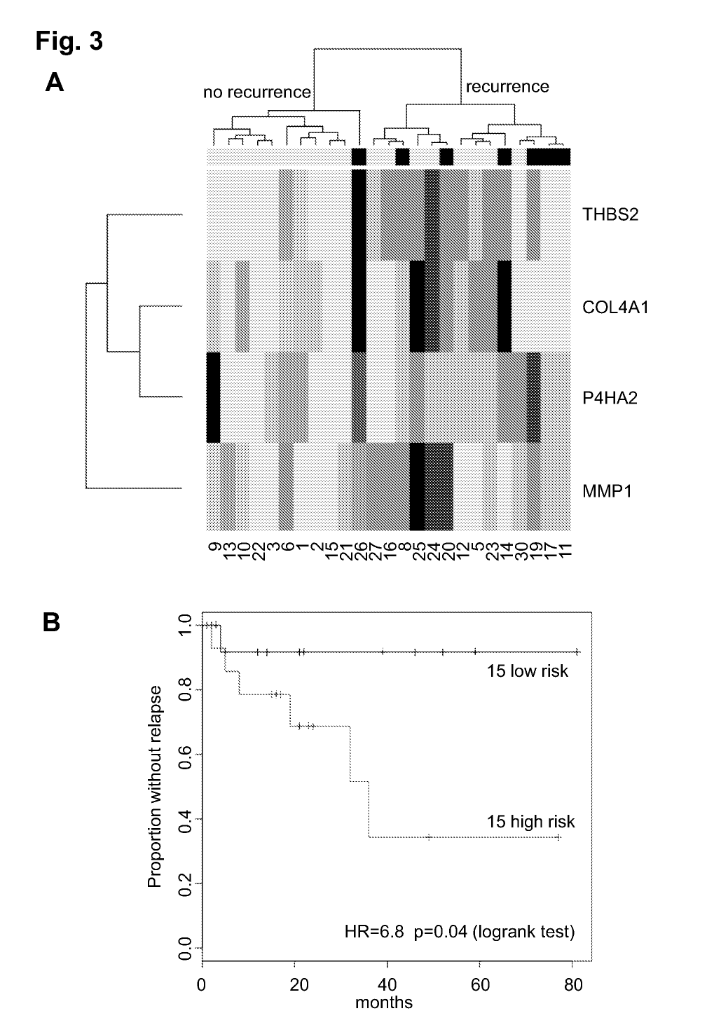

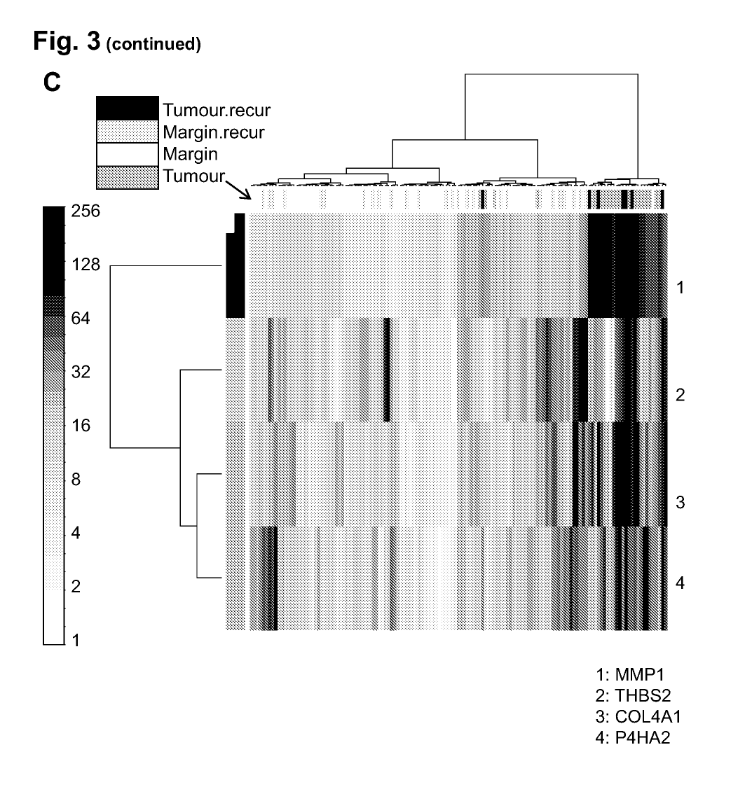

The 138 genes were subjected to penalized regression analysis, and results indicated a 4-gene signature (MMP1, COL4A1, P4HA2 and THBS2) predictive of OSCC recurrence. Quantitative PCR validation of this gene signature in a separate patient cohort (Table 2) confirmed that all 4 genes (MMP1, COL4A1, P4HA2 and THBS2) were up-regulated in margin and OSCC samples from patients with disease recurrence compared to margins and OSCCs from patients who did not recur (FIG. 3A) The dichotomized risk score was predictive of recurrence in the training cohort (89 samples; N=23 patients) (p=0.0003, logrank test) and in the independent test cohort (136 samples; N=30 patients) (HR=6.8, p=0.04, logrank test) (FIG. 3B). In addition, the dichotomized risk score improved on the predictive ability of T (tumor size) and N (nodal status) alone in multivariate Cox analysis (p=0.06, likelihood ratio test). Clinical variables, alone or in combination, were not predictive of recurrence in either training or validation cohorts. The coefficients of the 4-gene risk score, for use with z-score scaled expression values, are summarized in Table 6.

TABLE 6
Coefficients of the linear risk score for z-score normalized
log2-expression values. Fold-change (FC) is the geometric-average
expression in tumors relative to surgical resection margins.
P-values are for tumor/margin differential expression in the
qPCR (independent validation set) (Wilcoxon Rank Sum test)

| | | FC | FC (qPCR | p-value |
|---|---|---|---|---|
| Gene | Coefficient | (microarray) | validation) | (qPCR) |
| MMP1 | 0.63 | 405 | 798 | 9E−16 |
| COL4A1 | 0.25 | 3.7 | 4.3 | 7E−09 |
| P4HA2 | 0.45 | 2.7 | 2.8 | 1E−06 |
| THBS2 | 0.34 | 3 | 1.9 | 6E−03 |

TABLE 7
Exemplary accession and SEQ ID numbers of polynucleotide
and amino acid sequences for MMP1, COL4A1, P4HA2, THBS2,
PXDN and PMEPA1 (see Table 10 for sequences).

| | | Entrez | |
|---|---|---|---|
| | | Gene ID | |
| Gene | SEQ ID NO: | Number | Genbank ID |
| MMP1 | 11 and 12 | 4312 | NM_002421 |
| COL4A1 | 13 and 14 | 1282 | NM_001845 |
| P4HA2 | 15, 16 and 17 | 8974 | Variant 1: NM_004199, |
| | | | Variant 2: NM_001017973 |
| | | | Variant 3: NM_001017974 |
| | | | Variant 4: NM_001142598 |
| | | | Variant 5: NM_001142599 |
| THBS2 | 18 and 19 | 7058 | NM_003247 |
| PMEPA1 | 20 and 21 | 56937 | Variant 1: NM_020182.3 |
| | | | Variant 2: NM_199169 |
| | | | Variant 3: NM_199170 |
| | | | Variant 4: NM_199171 |
| PXDN | 22 and 23 | 7837 | NM_012293 |

### Effect of Reducing the Number of Available Margins

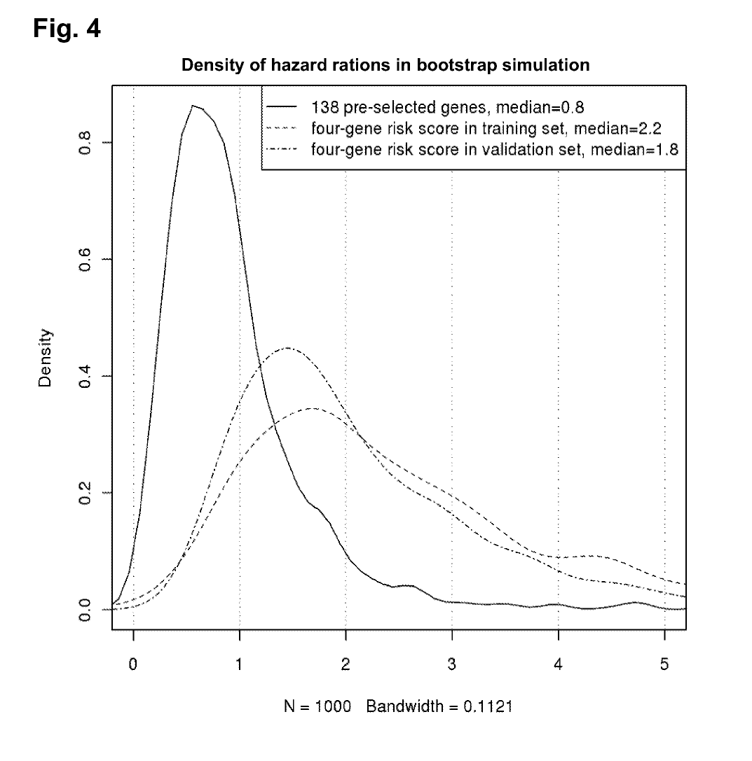

The simple mean of all 138 pre-selected genes does not show any prognostic effect in the bootstrap simulation of using only a single margin per patient (median HR=0.8) however, the 4-gene signature maintains an effect in both the training and validation sets (median HR=2.2 and 1.8, with 89% and 87% of bootstrapped hazard ratios greater than the no-effect value of HR=1 in the training and validation sets, respectively) (FIG. 4). Results from the bootstrap simulations showed smaller hazard ratios, compared with hazard ratios obtained when using the maximum expression value from several margins. These results suggest that up-regulated expression of these genes in a subset of margins best predicts recurrence and that sampling multiple margins improves the ability to detect recurrence risk.

### Discussion

It is known that histologically normal margins may harbor genetic changes also found in the primary tumor, as shown by studies in HNSCC, including oral carcinomas (7). In oral carcinoma, local recurrence may arise from cancer cells left behind after surgery, undetectable by histopathology (minimal residual cancer), or from fields of genetically altered cells with the potential to give rise to a new carcinoma (21); such fields precede the tumor and can be detected in the surrounding mucosa (surgical resection margins). Molecular changes that are commonly detected in margins as well as the corresponding tumor could indicate that pre-malignant or malignant clones were able to migrate to the surrounding tissue, giving rise to a primary tumor recurrence (22).

Herein, the significance of global gene expression analysis of histologically normal margins and OSCC as an approach for the identification of deregulated genes and pathways associated with OSCC recurrence is demonstrated. A multi-step procedure including an in-house whole-genome expression profiling experiment and a meta-analysis of five published microarray datasets was used to develop a robust 4-gene signature (MMP1, COL4A1, THBS2 and P4HA2) for prediction of recurrence in OSCC. This signature is based on genes found to be consistently over-expressed in OSCC as compared to normal oral mucosa; these genes are also over-expressed in a subset of histologically normal surgical resection margins, and their over-expression in such margins provides an indication of the presence of genetic changes before histological alterations can be detected by histology. Notably, the initial analyses reveal that this 4-gene signature predicted recurrence in two of the patients (Pts. 17 and 20, Table 2, validation set) who had not recurred until the latest update of the clinical data for recurrence status. Both of these patients had local recurrence, 8 and 19 months after surgery, respectively.

Genes identified in the 4-gene signature (MMP1, COL4A1, THBS2 and P4HA2) play major roles in cell-cell and/or cell-matrix interaction, and invasion. The direct and indirect partners of these genes are illustrated in FIG. 1. The functions of two genes (P4HA2 and THBS2) in the signature of OSCC recurrence and their roles in cancer are not well understood. P4HA2 encodes a key enzyme involved in collagen synthesis, and its over-expression has been previously reported in papillary thyroid cancer (23). THBS2 is a matricellular protein that encodes an adhesive glycoprotein and interacts with other proteins to modulate cell-matrix interactions (24). Interestingly, THBS2 is associated with tumor growth in adult mouse tissues (24). The two other genes in the OSCC recurrence signature (COL4A1 and MMP1) are better characterized in cancer. COL4A1 encodes the major type IV alpha collagen chain and is one of the main components of basement membranes. Basement membranes have several important biological roles, and are essential for embryonic development, proper tissue architecture, and tissue remodeling (25). COL4A1 binds other collagens (COL4A2, 3, 4, 5 and 6), as well as LAMC2 (laminin, gamma 2), TGFB1 (transforming growth factor, beta 1), among other proteins (FIG. 1) (http://www.ihop-net.org), playing a relevant role in extracellular matrix-receptor interaction and focal adhesion (26). The extracellular matrix undergoes constant remodeling; during this process, proteins such as MMP1 can degrade the extracellular matrix proteins (e.g., collagen IV), and contribute to invasion and metastasis (27). In cancer, combined over-expression of COL4A1 and LAMC2 can distinguish OSCC from clinically normal oral cavity/oropharynx tissues (28); this latter study suggests that COL4A1 over-expression may be a useful biomarker for early detection of malignancy.

MMP1 belongs to the family of matrix metalloproteases, which are key proteases involved in extracellular matrix (ECM) degradation (29). MMP1 encodes a collagenase, which is secreted by tumor cells as well as by stromal cells stimulated by the tumor; this secreted enzyme is responsible for breaking down interstitial collagens type I, II and III in normal physiological processes (e.g., tissue remodeling) as well as disease processes (e.g., cancer) (29). It is believed that the mechanism of up-regulation of most of the MMPs is likely due to transcriptional changes, which may occur following alterations in oncogenes and/or tumor suppressor genes (29).

In HNSCC, over-expression of several genes with roles in invasion and metastasis, including MMPs, were previously associated with treatment failure of HNSCC (30). In the present study, MMP1 was over-expressed in a subset of margins exclusively from patients with recurrent OSCC, and showed the highest fold-change of up-regulation in OSCC compared to margins. These results support the notion that MMP1 may be involved in initial steps of tumorigenesis as well as invasion of oral carcinoma cells. Indeed, matrix metalloproteinases play an important role not only in invasion and metastasis but also in early stages of cancer development/progression, reviewed in (29).

The data suggests that histologically normal surgical resection margins that over-express MMP1, COL4A1, THBS2 and P4HA2 are indicative of an increased risk of recurrence in OSCC. Patients at higher risk of recurrence could potentially benefit from closer disease monitoring and/or adjuvant post-operative radiation treatment, even in the absence of other clinical and histopathological indicators, such as advanced disease stage and perineural invasion. Since this 4-gene signature was predictive of recurrence in two separate patient cohorts, over-expression of this signature may be used for molecular analysis of histologically negative margins, and may improve recurrence risk assessment in patients with OSCC.

### Example 2

In the clinic, genetic analysis of histologically normal margins can be performed to determine the expression of the 4-gene signature.

This analysis can be done after surgery, using either the frozen margins or the formalin-fixed, paraffin-embedded (FFPE) margin tissues. It is likely to use these FFPE tissues, since fixation in formalin and paraffin-embedding is a standard procedure for these samples.

In this case, qRT-PCR or digital molecular barcoding technology, such as Nanostring analysis of these tissues could be used.

Following genetic analysis, a risk score can be calculated which indicates the risk of the patient to have recurrence of the primary tumor. The risk score is a weighted average of expression values, using the coefficients provided in Table 6. For example, the relative expression of each gene, relative to the control sample and optionally one or more endogenous control genes (such as GAPDH, actin etc is calculated and used to calculate a value of the risk score for the subject using a weighted average given by the coefficients in Table 6. On the basis of this continuous risk score, the subject can be given a good or bad prognosis as determined by comparing the risk score to a predetermined threshold. This risk score can also be divided into low, moderate or high, using two predetermined thresholds. Thresholds are predetermined using a population with known outcome, such as those in this study, or for example from a prospective clinical trial. The clinician/surgeon responsible for the patient should be able to advise closer follow-up or adjuvant radiation therapy, for example, for a patient with higher risk of recurrence.

### Example 3

The predictive ability of all subsets of the four-gene signature in the training and validation cohorts was estimated by bootstrap resampling of a single margin per patient. For each simulation, a single margin from each patient was selected randomly and used to calculate the risk score for that patient. These risk scores were used to estimate a hazard ratio for each simulation. The results are shown in Table 8. Median HR is the median hazard ratio of the thousand simulations, and fraction >1 is the fraction of simulations where the estimated hazard ratio was greater than 1 (some predictive effect). Only two subsets in the validation set were not estimated to have predictive value (COL4A1 and THBS2+COL4A1). For example, the THBS2+COL4A1 combination is likely not predictive due to the contribution of COL4A1.

TABLE 8
Predictive ability of all subsets of the four-gene signature
in the training and validation cohorts, estimated by bootstrap
resampling of a single margin per patient

| | training | | validation | |
|---|---|---|---|---|
| | | fraction > | | fraction > |
| signature | median HR | 1 | median HR | 1 |
| MMP1 | 1.522185551 | 0.766 | 1.225218311 | 0.668 |
| P4HA2 | 1.725695969 | 0.819 | 1.098933192 | 0.673 |
| THBS2 | 1.746312863 | 0.794 | 1.204582762 | 0.651 |
| COL4A1 | 1.325996586 | 0.699 | 0.813809208 | 0.188 |
| MMP1, P4HA2 | 1.699798301 | 0.878 | 1.267399811 | 0.772 |
| MMP1, THBS2 | 1.542774823 | 0.751 | 1.315878037 | 0.763 |
| MMP1, COL4A1 | 1.746312863 | 0.831 | 1.192355867 | 0.67 |
| P4HA2, THBS2 | 1.333344112 | 0.665 | 1.098933192 | 0.608 |
| P4HA2, COL4A1 | 1.947785623 | 0.903 | 1.047778866 | 0.591 |
| THBS2, COL4A1 | 1.480921222 | 0.75 | 0.890808881 | 0.341 |
| MMP1, P4HA2, | 1.387380595 | 0.715 | 1.320252399 | 0.769 |
| THBS2 | | | | |
| MMP1, P4HA2, | 1.594163223 | 0.829 | 1.253103413 | 0.772 |
| COL4A1 | | | | |
| MMP1, THBS2, | 1.63372399 | 0.82 | 1.334546396 | 0.761 |
| COL4A1 | | | | |
| P4HA2, THBS2, | 1.480921222 | 0.727 | 1.070331384 | 0.627 |
| COL4A1 | | | | |
| MMP1, P4HA2, | 1.655600711 | 0.795 | 1.283925403 | 0.77 |
| THBS2, COL4A1 | | | | |

### Example 4

Gene expression levels can be detected using digital molecular barcoding technologies such as Nanostring nCounter using for example the following probes.

TABLE 9
Probe sequences for Digital Molecular Barcoding Technology

| | | Target | | |
|---|---|---|---|---|
| | | Region | | |
| | | within | | |
| | | the | Probe sequence for Digital Barcoding | SEQ ID |
| Gene | Nucleotide ID | gene | Technology | NO: |
| MMP1 | NM_002421.3 | 1117- | AAATGGGCTTGAAGCTGCTTACGAATTTGCCGAC | 24 |
| | | 1217 | AGAGATGAAGTCCGGTTTTTCAAAGGGAATAAGT | |
| | | | ACTGGGCTGTTCAGGGACAGAATGTGCTACAC | |
| COL4A1 | NM_001845.4 | 780- | TGGGCTTAAGTTTTCAAGGACCAAAAGGTGACAA | 25 |
| | | 880 | GGGTGACCAAGGGGTCAGTGGGCCTCCAGGAG | |
| | | | TACCAGGACAAGCTCAAGTTCAAGAAAAAGGAGA | |
| P4HA2 | NM_001017974.1 | 1600- | TGTGCTTGTGGGCTGCAAGTGGGTCTCCAATAAG | 26 |
| | | 1700 | TGGTTCCATGAACGAGGACAGGAGTTCTTGAGAC | |
| | | | CTTGTGGATCAACAGAAGTTGACTGACATCCT | |
| THBS2 | NM_003247.2 | 4460- | AAACATCCTTGCAAATGGGTGTGACGCGGTTCCA | 27 |
| | | 4560 | GATGTGGATTTGGCAAAACCTCATTTAAGTAAAA | |
| | | | GGTTAGCAGAGCAAAGTGCGGTGCTTTAGCTG | |

### Example 5

TABLE 10
MMP1
Official Symbol: MMP1 and Name: matrix metallopeptidase 1 (interstitial
collagenase) [Homo sapiens]
Other Aliases: CLG, CLGN
Other Designations: fibroblast collagenase; interstitial collagenase; matrix
metalloprotease 1
Chromosome: 11; Location: 11q22.3
Annotation: Chromosome 11, NC_000011.9 (102660651 . . . 102668894,
complement)
MIM: 120353
Gene ID: 4312
Nucleotide ID (isoform 1 and isoform 2): NM_002421
>gi|225543092|ref|NM_002421. 3| Homo sapiens matrix
metallopeptidase 1 (interstitial collagenase) (MMP1), transcript variant 1,
mRNA|
SEQ ID NO: 11
Protein sequence (MMP1) length = 403| SEQ ID NO: 12
THBS2
Official Symbol: THBS2 and Name: thrombospondin 2 [Homo sapiens]
Other Aliases: XXyac-YX65C7_A.1, TSP2
Other Designations: thrombospondin-2
Chromosome: 6; Location: 6q27
Annotation: Chromosome 6, NC_000006.11 (169615875 . . . 169654137,
complement)
MIM: 188061
Gene ID: 7058
Nucleotide ID: NM_003247
>gi|40317627|ref|NM_003247.2| Homo sapiens thrombospondin 2
(THBS2), mRNA|
SEQ ID NO: 18
Protein sequence (THBS2) length = 1172| SEQ ID NO: 19
P4HA2
Official Symbol: P4HA2 and Name: prolyl 4-hydroxylase, alpha
polypeptide II [Homo sapiens]
Other Aliases: UNQ290/PRO330
Other Designations: 4-PH alpha 2; 4-PH alpha-2; C-P4Halpha(II);
OTTHUMP00000065969; collagen prolyl 4-hydroxylase alpha(II);
procollagen-proline, 2-oxoglutarate 4-dioxygenase (proline 4-
hydroxylase), alpha polypeptide II; procollagen-proline, 2-oxoglutarate-
4-dioxygenase subunit alpha-2; prolyl 4-hydroxylase
subunit alpha-2
Chromosome: 5; Location: 5q31
Annotation: Chromosome 5, NC_000005.9 (131528303 . . . 131563556,
complement)
MIM: 600608
Gene ID: 8974
Nucleotide ID:
prolyl 4-hydroxylase, alpha II subunit transcript variant 1: NM_004199
prolyl 4-hydroxylase, alpha II subunit transcript variant 2:
NM_001017973
prolyl 4-hydroxylase, alpha II subunit transcript variant 3:
NM_001017974
prolyl 4-hydroxylase, alpha II subunit transcript variant 4:
NM_001142598
prolyl 4-hydroxylase, alpha II subunit transcript variant 5:
NM_001142599
>gi|63252890|ref|NM_001017973.1|Homo sapiens prolyl 4-
hydroxylase, alpha polypeptide II (P4HA2), transcript variant 2, mRNA|
SEQ ID NO: 15
Protein sequence (P4HA2, isoform 1) length = 535|
SEQ ID NO: 16
Protein sequence (P4HA2, isoform 2) length = 533|
SEQ ID NO: 17
COL4A1
Official Symbol: COL4A1 and Name: collagen, type IV, alpha 1 [Homo
sapiens]
Other Aliases: arresten
Other Designations: COL4A1 NCI domain; OTTHUMP00000194462;
collagen IV, alpha-1 polypeptide; collagen alpha-1 (IV) chain;
collagen of basement membrane, alpha-1 chain
Chromosome: 13; Location: 13q34
Annotation: Chromosome 13, NC_000013.10 (110801310 . . .
110959496, complement)
MIM: 120130
Gene ID: 1282
Nucleotide ID: NM_001845
>gi|148536824|ref|NM_001845.4| Homo sapiens collagen, type
IV, alpha 1 (COL4A1), mRNA|
SEQ ID NO: 13
Protein sequence (COL4A1) length = 1669|
SEQ ID NO: 14
PMEPA1
Official Symbol: PMEPA1 and Name: prostate transmembrane protein,
androgen induced 1 [Homo sapiens]
Other Aliases: STAG1, TMEPAI
Other Designations: OTTHUMP00000174283; OTTHUMP00000174284;
solid tumor-associated 1 protein; transmembrane prostate androgen-
induced protein; transmembrane, prostate androgen induced RNA
Chromosome: 20; Location: 20q13.31-q13.33
Annotation: Chromosome 20, NC_000020.10 (56223452 . . . 56286541,
complement)
MIM: 606564
Gene ID: 56937
Nucleotide ID:
Transcript variant 1: NM_020182.3
Transcript variant 2: NM_199169
Transcript variant 3: NM_199170
Transcript variant 4: NM_19917l
NM_020182.3| GI: 40317614| Homo sapiens prostate transmembrane
protein, androgen induced 1 (PMEPA1), transcript variant 3, mRNA|
SEQ ID NO: 20
Homo sapiens prostate transmembrane protein, androgen induced 1
(PMEPA1), transcript variant 3| Protein (PMEPA1) length = 237|
SEQ ID NO: 21
PXDN
Official Symbol: PXDN and Name: peroxidasin homolog (Drosophila)
[Homo sapiens]
Other Aliases: D2S448, D2S448E, KIAA0230, MG50, PRG2, PXN, VPO
Other Designations: OTTHUMP00000199943; melanoma-associated
antigen MG50; p53-responsive gene 2 protein; peroxidasin homolog;
vascular peroxidase 1, peroxidasin precursor
Chromosome: 2; Location: 2p25
Annotation: Chromosome 2, NC_000002.11 (1635659 . . . 1748291,
complement)
MIM: 605158
Gene ID: 7837
Nucleotide ID: NM_012293
NM_012293.1| GI: 109150415| Homo sapiens peroxidasin homolog
(Drosophila) (PXDN), mRNA|
SEQ ID NO: 22
Protein (PXDN) length = 1479|
SEQ ID NO: 23

### Example 6

Background:

A recently developed probe-based technology, the NanoString nCounter™ gene expression system, has been shown to allow accurate mRNA transcript quantification using low amounts of total RNA. The ability of this technology was assessed for mRNA expression quantification in archived formalin-fixed, paraffin-embedded (FFPE) oral carcinoma samples.

Results:

The mRNA transcript abundance of 20 genes (COL3A1, COL4A1, COL5A1, COL5A2, CTHRC1, CXCL1, CXCL13, MMP1, P4HA2, PDPN, PLOD2, POSTN, SDHA, SERPINE1, SERPINE2, SERPINH1, THBS2, TNC, GAPDH, RPS18) in 38 samples (19 paired fresh-frozen and FFPE oral carcinoma tissues, archived from 1997-2008) by both NanoString and SYBR Green I fluorescent dye-based quantitative real-time PCR(RQ-PCR). The gene expression data obtained by NanoString vs. RQ-PCR in both fresh-frozen and FFPE samples was compared. Fresh-frozen samples showed a good overall Pearson correlation of 0.78, and FFPE samples showed a lower overall correlation coefficient of 0.59, which is likely due to sample quality. A higher correlation coefficient between fresh-frozen and FFPE samples analyzed by NanoString (r=0.90) compared to fresh-frozen and FFPE samples analyzed by RQ-PCR (r=0.50). In addition, NanoString data showed a higher mean correlation (r=0.94) between individual fresh-frozen and FFPE sample pairs compared to RQ-PCR (r=0.53).

Conclusions:

Based on these results, both technologies can be used for gene expression quantification in fresh-frozen or FFPE tissues. The probe-based NanoString method achieved superior gene expression quantification results when compared to RQ-PCR in archived FFPE samples. This newly developed technique would seem to be optimal for large-scale validation studies using total RNA isolated from archived, FFPE samples.

### Background

A vast collection of formalin-fixed and paraffin-embedded (FFPE) tissue samples are currently archived in anatomical pathology laboratories and tissue banks around the world. These samples are an extremely valuable source for molecular biology studies, since they have been annotated with varied information on disease states and patient follow-up, such as disease progression in cancer and prognosis/survival data. Although FFPE samples provide an ample source for genetic studies, formalin fixation is known to affect the quality of DNA and RNA extracted from FFPE samples and its downstream applications, such as amplification by the Polymerase Chain Reaction (PCR) or microarrays [51].

Von Ahlfen et al., 2007 [51] described the different factors (e.g. fixation, storage time and conditions) that can influence the integrity of RNA extracted from FFPE tissues, and its downstream applications. They showed that differences in storage time and temperature had a large effect on the degree of RNA degradation. In their study, RNA samples extracted within 1 to 3 days after formalin fixation and paraffin embedding maintained their integrity. Similarly, RNA isolated from FFPE samples that were stored at 4° C. showed higher quality compared to samples stored at room temperature or at 37° C. They also reported that RNA fragmentation occurs gradually over time. It is also known that cDNA synthesis from FFPE-derived RNA is limited due to the use of formaldehyde during fixation. Formaldehyde induces chemical modification of RNA, characterized by the formation of methylene crosslinks between nucleic acids and protein. These chemical modifications can be partially irreversible [52], limiting the application of techniques such as reverse transcription, which uses mRNA as template for cDNA synthesis. A fixation time over 24 hours was shown to result in a higher number of irreversible crosslinks [53, 54]. Overall, fixation time and method of RNA extraction are the main factors that determine the extent of methylene crosslinks [51].

A recently developed probe-based technology, the NanoString nCounter™ gene expression system, has been shown to allow accurate mRNA expression quantification using low amounts of total RNA [55]. This technique is based on direct measurement of transcript abundance, by using multiplexed, color-coded probe pairs, and is able to detect as little as 0.5 fM of mRNA transcripts; described in detail in Geiss et al., 2008 [55]. In brief, unique pairs of a capture and a reporter probe are synthesized for each gene of interest, allowing ˜800 genes to be multiplexed, and their mRNA transcript levels measured, in a single experiment, for each sample. In addition, in a recent study, mRNA expression levels obtained using NanoString were more sensitive than microarrays and yielded similar sensitivity when compared to two quantitative real-time PCR techniques: TaqMan-based RQ-PCR and SYBR Green I fluorescent dye-based RQ-PCR [55]. Although NanoString and RQ-PCR were shown to produce comparable data in good quality samples, NanoString is hybridization-based, and does not require reverse transcription of mRNA and subsequent cDNA amplification. This feature of NanoString technology offers advantages over PCR-based methods, including the absence of amplification bias, which may be higher when using fragmented RNA isolated from FFPE specimens. In addition, NanoString assays do not require the use of assay control samples, since absolute transcript abundance is determined for each single sample and normalized against the expression of housekeeping genes in that same sample [55].

Although NanoString technology has been optimized for gene expression analysis using formalin-fixed samples, to our knowledge this is the first report of the use of this technology for mRNA transcript quantification using clinical, archival, FFPE cancer tissues. In the pilot study, the NanoString nCounter™ assay was used for gene expression analysis of archival oral carcinoma samples. In order to show that mRNA levels obtained by NanoString analysis of FFPE tissues were accurate, quantification data obtained using RNA isolated from paired fresh-frozen and FFPE oral cancer samples were compared. The goal was to determine whether this technology could be applied for accurate gene expression quantification using archived, FFPE oral cancer tissues. It was also sought to compare whether quantification data obtained by NanoString achieved a higher correlation than data obtained by SYBR Green I fluorescent dye-based RQ-PCR, using the same paired fresh-frozen and FFPE samples.

### Methods

### Tissue Samples

This study was performed under approval of the Research Ethics Board at University Health Network. Tissues were collected with informed patient consent. Study samples included primary fresh-frozen and formalin-fixed, paraffin-embedded (FFPE) tumor samples from 19 patients with oral squamous cell carcinoma. All patients had surgery as primary treatment. Fresh-frozen tissues were collected at the time of surgical resection, and samples were snap frozen and kept in liquid nitrogen until RNA extraction. RNA from these tumor samples was extracted and kept at −80C for long term storage. Representative FFPE tissue sections were obtained from the same tumor samples. A total of 38 tumor samples (paired fresh-frozen and FFPE) from 19 patients were collected. In addition, a commercially available human universal RNA (pool of cancer cell lines) (Stratagene) and human normal tongue RNA (Stratagene) were analysed; these samples were used as quality controls, since they are a source of high quality RNA, and have been previously used in other studies [56, 57].

RNA Extraction and cDNA Synthesis

Total RNA was isolated from fresh-frozen tissues using Trizol reagent (Life Technologies, Inc., Burlington, ON, Canada), followed by purification using the Qiagen RNeasy kit and treatment with the DNase RNase-free set (Qiagen, Valencia, Calif., USA). RNA extraction and purification steps were performed according to the manufacturers' instructions.

For FFPE tissue, one tissue section was taken from each specimen, prior to RNA extraction, stained with hematoxylin and eosin (H&E) and examined by a pathologist (B.P-O), to ensure that tissues contained >80% tumor cells. RNA was isolated from five 10 μm sections from FFPE samples, using the RecoverAll™ Total Nucleic Acid Isolation Kit (Ambion, Austin, Tex., USA), following the manufacturer's procedures. RNA extracted from both fresh-frozen and FFPE tissues was assessed for quantity using Nanodrop 1000 (Nanodrop), and for quality using the 2100 Bioanalyzer (Agilent Technologies, Canada).

For RQ-PCR experiments, cDNA was synthesized from 1 μg total RNA isolated from fresh-frozen or FFPE tissues, using the M-MLV reverse transcriptase enzyme and according to manufacturer's protocol (Invitrogen).

Gene Expression Quantification Using Multiplexed, Color-Coded Probe Pairs (NanoString nCounter™)

Genes selected for testing in this technical report are frequently over-expressed in oral cancer (70, 71).

Probe sets for each gene were designed and synthesized by NanoString nCounter™ technologies (Table 11). Probe sets of 100 bp in length were designed to hybridize specifically to each mRNA target. Probes contained one capture probe linked to biotin and one reporter probe attached to a color-coded molecular tag, according to the nCounter™ code-set design.

RNA samples were randomized using a numerical ID, in order to blind samples for sample type (fresh-frozen or FFPE) and sample pairs. Samples were then subjected to NanoString nCounter™ analysis by the University Health Network Microarray Centre (http://www.microarrays.ca/) at the Medical Discovery District (MaRS), Toronto, ON, Canada. The detailed protocol for mRNA transcript quantification analysis, including sample preparation, hybridization, detection and scanning followed the manufacturer's recommendations, and are available at http://www.nanostring.com/uploads/Manual_Gene_Expression_Assay.pdf/ under http://www.nanostring.com/applications/subpage.asp?id=343. (72) A 100 ng of total RNA isolated from fresh-frozen tissues was used, as suggested by the manufacturer. FFPE tissues required a higher amount of total RNA (400 ng) for detection of probe signals. Technical replicates of three paired fresh-frozen and FFPE tissues were included. Data were analyzed using the nCounter™ digital analyzer software, available at http://www.nanostring.com/support/ncounter/.

### Quantitative Real-Time RT-PCR

In addition, RQ-PCR analysis was performed in the same fresh-frozen and FFPE samples and compared to gene expression data determined by NanoString nCounter assay. RQ-PCR analysis was performed as previously described, using SYBR Green I fluorescent dye [58, 59]. Gene IDs and primer sequences are described in Table 12. Primer sequences were designed using Primer-BLAST (http://www.ncbi.nlm.nih.gov/tools/primer-blast/). Gene expression levels were normalized against the average Ct (cycle threshold) values for the two internal control genes (GAPDH and RPS18) and calculated relative to a commercially available normal tongue reference RNA (Stratagene). Ct values were extracted using the SDS 2.3 software (Applied Biosystems). Data analysis was performed using the delta delta Ct method [60].

### Statistical Analysis

Absolute mRNA quantification values obtained by NanoString as well as relative expression values obtained by RQ-PCR were log 2-transformed. Summary statistics as median, mean, range were provided. Pair-wise Pearson product-moment correlation analysis [61] was applied to test the correlation between gene expression data obtained by NanoString and RQ-PCR analysis in fresh-frozen vs. FFPE samples, as well as the correlation between NanoString and RQ-PCR data in fresh-frozen or FFPE samples. Both overall correlation and correlation across sample pairs were calculated. Statistical analyses were performed using version 9.2 of the SAS system and user's guide (SAS Institute, Cary, N.C.). In addition, Pearson correlation between sample pairs was plotted as heatmaps, in order to visualize the grouping of similar samples. Heatmaps were generated by hierarchical clustering analysis, using hclust R function, in R statistical environment [62].

### Results

### Technical Data on Sample Quality

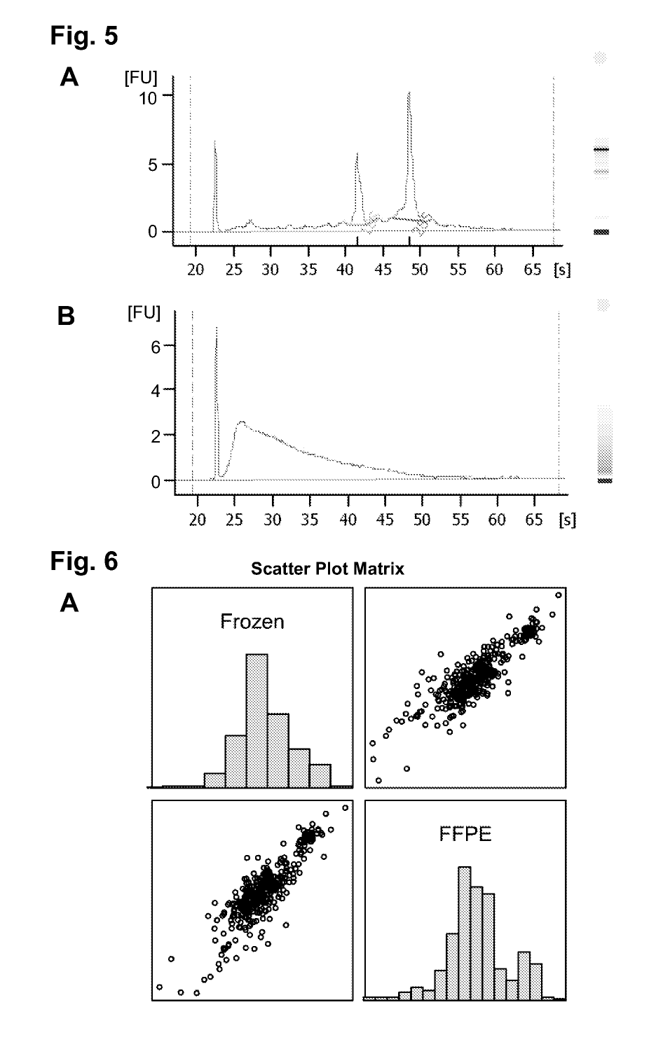

Bioanalyzer results for fresh-frozen samples showed a mean RNA integrity number (RIN) of 8.3 (range 4.6-9.8), with the majority of fresh-frozen samples (13/19) having a RIN≧8. FFPE samples were degraded and the mean RIN was 2.3 (range 1.5-2.5); this result was expected since FFPE samples are archival tissues. Representative examples of the Bioanalyzer results for one fresh-frozen and one FFPE sample are shown in FIG. 5. FFPE samples used in the study have been archived from a time period between 1997-2008.

Correlation Between mRNA Transcript Quantification in Fresh-Frozen Vs. FFPE Samples (NanoString)

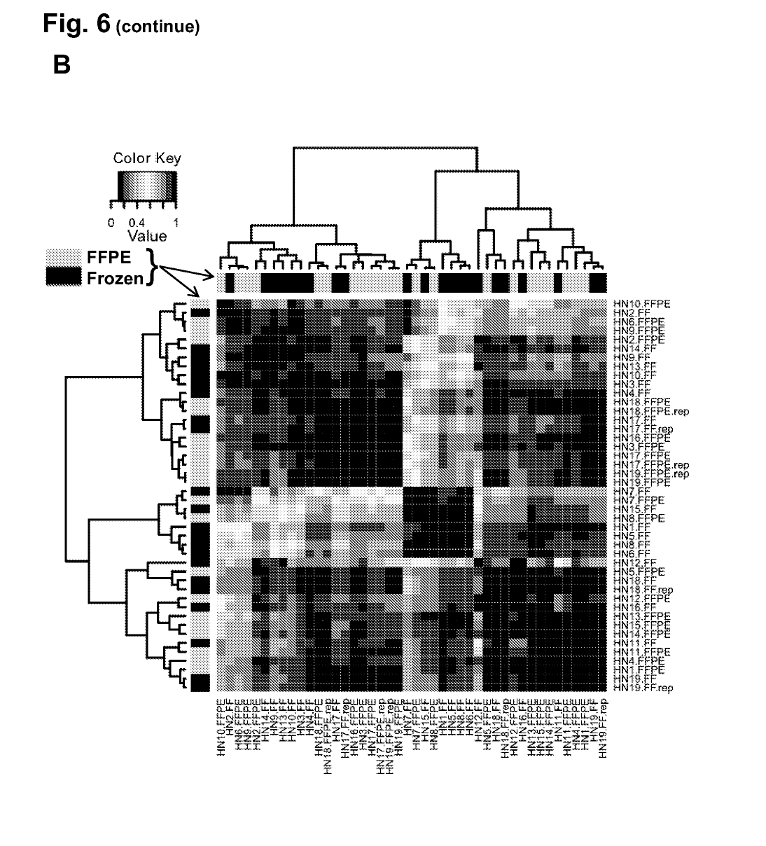

Raw data quantification values obtained by NanoString were log 2 transformed, and values derived from the 19 paired fresh-frozen and FFPE samples were compared. The pair-wise Pearson product-moment correlation was 0.90 (p<0.0001). The scatter plot and histogram for log 2 values from fresh-frozen and FFPE samples are shown in FIG. 6A. Analysis of the three replicate pairs (log 2 transformed values) demonstrated a correlation of 0.93 (p<0.0001). In addition, unsupervised hierarchical clustering analysis of these data was performed, and heatmaps are shown in FIG. 6B.

A correlation analysis was also performed between mRNA transcript quantification values (log 2 transformed values) for each pair of fresh-frozen versus FFPE sample (sample by sample comparison). This analysis is important as it allows us to determine whether the amount of mRNA transcripts of a given gene is maintained in individual sample pairs. The mean correlation coefficient obtained was 0.94, with a minimum correlation of 0.77 and a maximum correlation of 0.99.

Correlation Between Gene Expression Levels in Fresh-Frozen Vs. FFPE Samples (RQ-PCR)

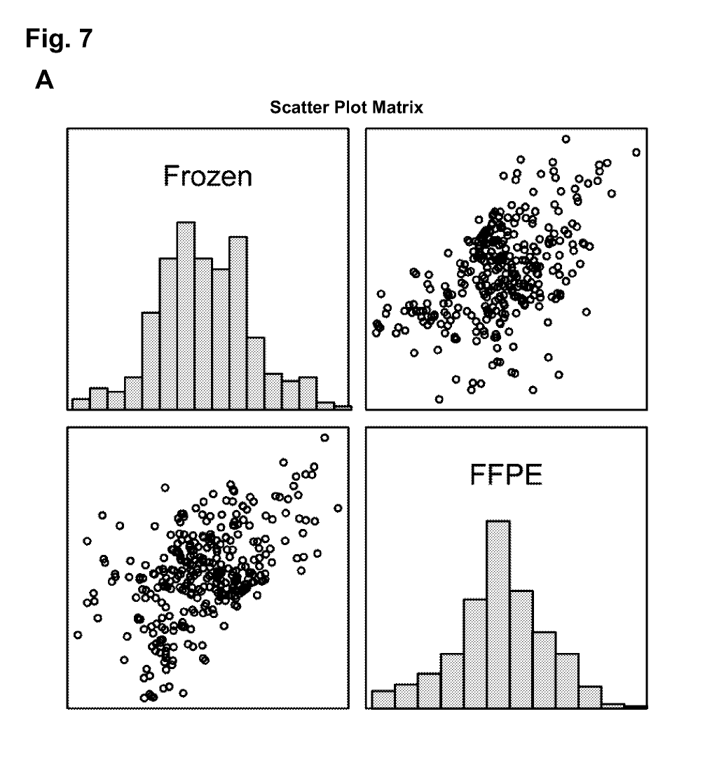

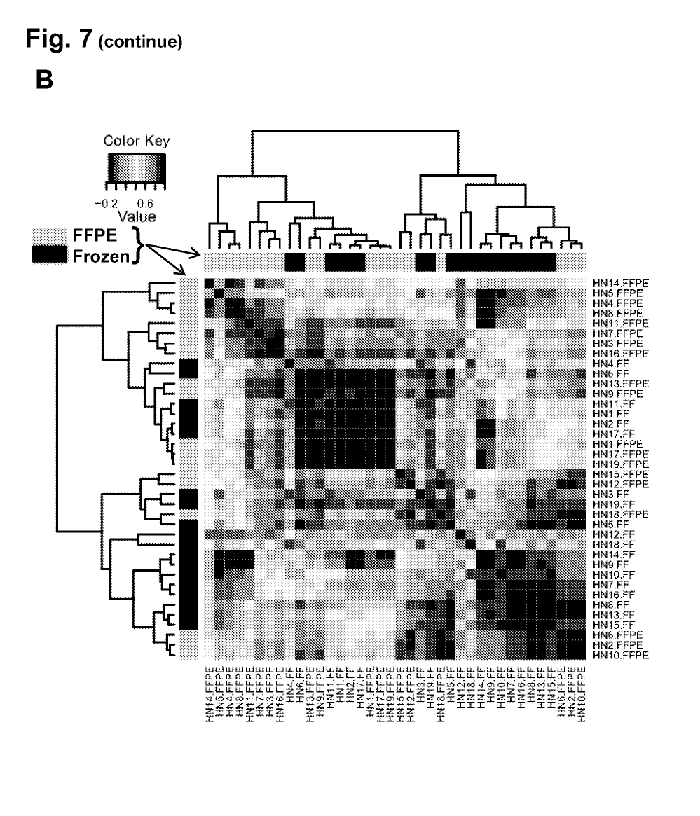

The gene expression levels determined by RQ-PCR analysis in fresh-frozen versus FFPE samples were also compared. The overall pair-wise Pearson product-moment correlation coefficient was 0.53 (p<0.0001) (FIG. 7A). Heatmap analysis of these data is shown in FIG. 7B. A sample-by-sample (fresh-frozen/FFPE sample pair) correlation analysis of RQ-PCR data revealed a mean correlation of 0.54, variable between 0.12 and 0.99, with the majority of sample pairs (12/19) showing a correlation ≧0.50.

Comparison of mRNA Quantification Data Using NanoString Versus RQ-PCR

Since all RNA samples isolated from FFPE tissues were degraded, as confirmed by Bioanalyzer analysis, it was expected that a probe-based assay would generate more accurate gene expression quantification data compared to amplification-based assays, such as RQ-PCR.

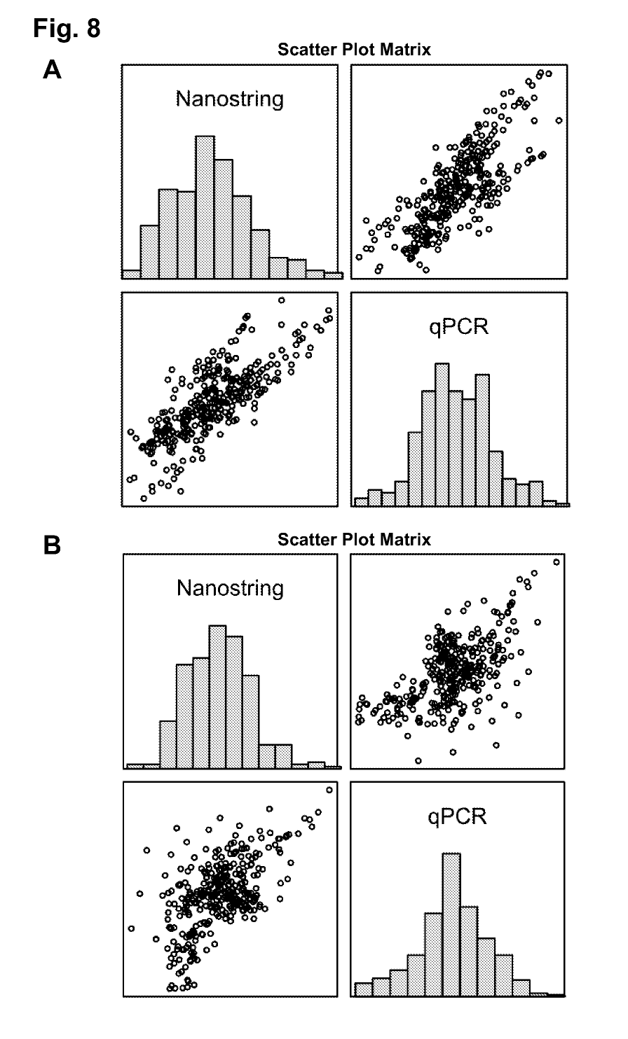

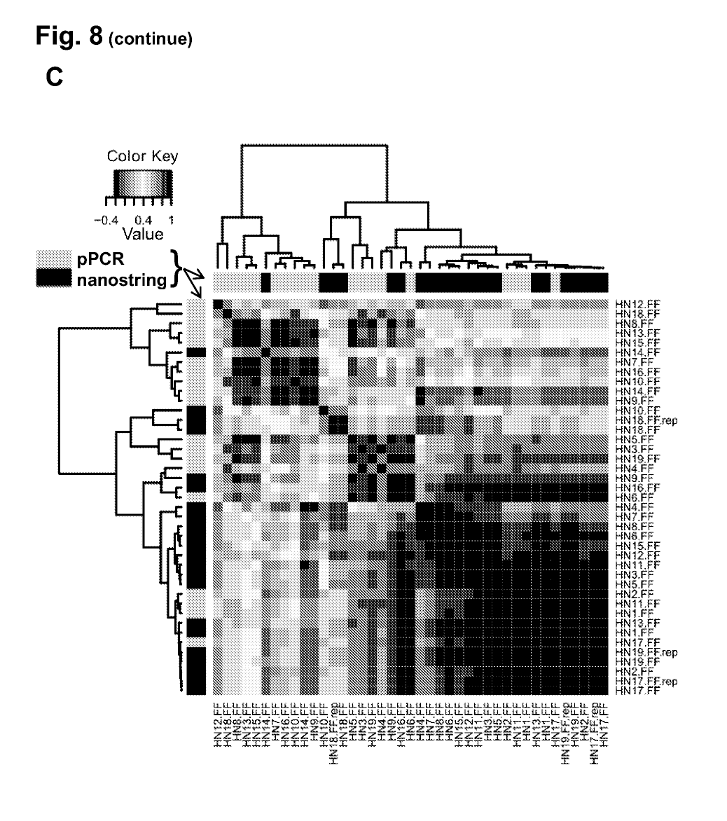

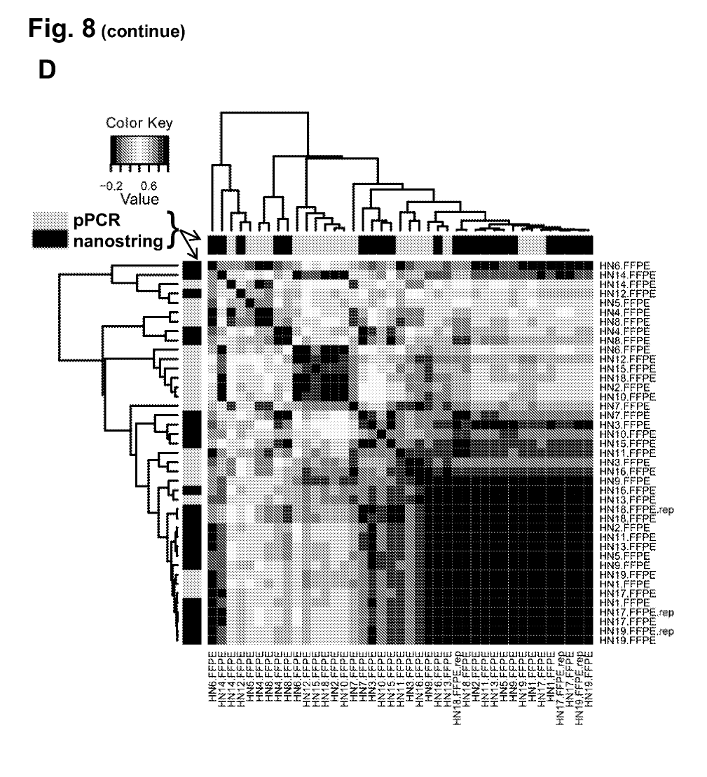

For each sample type (fresh-frozen or FFPE), mRNA transcript quantification as determined by NanoString analysis and gene expression levels as determined by RQ-PCR were compared. For fresh-frozen tissues, this comparison analysis showed that the overall pair-wise Pearson product-moment correlation coefficient was 0.78 (p<0.0001). FIG. 8A shows the scatter plot for the Log(NanoString) vs. Log(QPCR) and their histogram in fresh-frozen tissues. This same analysis in FFPE samples showed a lower overall correlation coefficient of 0.59 (p<0.0001); 11/19 FFPE sample pairs showed a correlation ≧0.60. FIG. 8B shows the scatter plot for the Log(NanoString) vs. Log(QPCR) and their histogram in FFPE tissues. Unsupervised hierarchical clustering analysis of these data was performed and corresponding heatmaps are shown in FIG. 8C, 8D.

### Discussion

In this pilot study, it was demonstrated that NanoString technology is suitable for accurately detecting and measuring mRNA transcript levels in clinical, archival, FFPE oral carcinoma samples. The results demonstrated that this probe-based assay (NanoString) achieved a good overall Pearson correlation when compared to mRNA transcript quantification results between paired fresh-frozen and FFPE samples. In addition, correlation coefficients were determined in a sample-by-sample comparison, and results showed that mRNA levels in single sample pairs (fresh-frozen and FFPE) was maintained across the sample pairs when using NanoString technology. When gene expression levels obtained by RQ-PCR were compared, a lower overall correlation coefficient was obtained between fresh-frozen and FFPE tissues, and across sample pairs. These results suggest that mRNA transcript levels are more concordant between fresh-frozen and FFPE sample pairs when using NanoString technology.

A recently published study [63] evaluated the performance of quantitative real-time PCR using TaqMan assays (TaqMan Low Density Arrays platform), for gene expression analysis using paired fresh-frozen and FFPE breast cancer samples. The investigators found a good overall correlation coefficient of 0.81 between fresh-frozen and FFPE samples; however, when they compared individual sample pairs, they found a low correlation of 0.33, with variability of 0.005-0.81. These authors suggested that the extensive RNA sample degradation in FFPE samples is likely the cause for the low correlation coefficients observed across sample pairs [63]. Indeed, Bioanalyzer results for our samples showed that fresh-frozen tissues had a good quality RNA Integrity Number (RIN) and were suitable for gene expression analysis, while FFPE tissues were degraded and had a low RIN. This RNA degradation in FFPE samples also resulted in higher Ct values initially detectable by RQ-PCR, with loss of amplifiable templates. The low RIN characteristic of FFPE samples did not seem to have an effect on the efficiency of NanoString results, however, when quantification values obtained using RNA isolated from fresh-frozen vs. FFPE tissues were compared.

Although quantitative PCR-based assays have been used for gene expression analysis in FFPE samples [63-65], these assays do carry some disadvantages, such as the need for optimization strategies aiming at reducing amplification bias and increasing the number of detectable amplicons when using RNA extracted from FFPE samples. To date, some of the recommended strategies include optimization of the RNA extraction method and designing primers able to detect short amplicons [66]. In the present study, primers for RQ-PCR experiments yielded amplicon lengths between 72-170 bp (as detailed in Table 12). Only 2/19 primer pairs yield amplicons >110 bp in size. Such short amplicons are well-suited for PCR amplification using FFPE samples. The results showed that, gene expression data using RQ-PCR can be obtained in FFPE samples, both the overall and the sample-by-sample correlation between fresh-frozen and FFPE samples was notably lower for RQ-PCR data than data obtained using NanoString. This suggests that this newly developed technology, NanoString nCounter™, offers advantages over RQ-PCR for gene expression analysis in archival FFPE samples.

### CONCLUSIONS

A multiplexed, color-coded probe-based method (NanoString nCounter™) achieved superior gene expression quantification results when compared to RQ-PCR, when using total RNA extracted from clinical, archival, FFPE samples. Such technology could thus be very useful for applications requiring the use of clinical archival material, such as large scale validation of gene expression data generated by microarrays for generation of tissue specific gene expression signatures.

### LIST OF ABBREVIATIONS

Ct: cycle threshold; FFPE: formalin fixed, paraffin embedded; H&E: hematoxylin and eosin; M-MLV RT enzyme: Moloney Murine Leukemia Virus reverse transcriptase enzyme; PCR: polymerase chain reaction; RIN: RNA integrity number; RQ-PCR: Quantitative real-time PCR; SAS: Statistical analysis system; SDS: Sequence Detection System

TABLE 11
Probe sets for genes of interest used for Nanostring analysis

| Gene | Accession | Target | |
|---|---|---|---|
| Symbol | Number | Region | Target Sequence |
| COL3A1 | NM_000090.3 | 180-280 | TTGGCACAACAGGAAGCTGTTGAAGGAGGATGTTCCCAT |
| | | | CTTGGTCAGTCCTATGCGGATAGAGATGTCTGGAAGCCA |
| | | | GAACCATGCCAAATATGTGTCT (SEQ ID NO: 28) |
| COL4A1 | NM_001845.4 | 780-880 | TGGGCTTAAGTTTTCAAGGACCAAAAGGTGACAAGGGTG |
| | | | ACCAAGGGGTCAGTGGGCCTCCAGGAGTACCAGGACAA |
| | | | GCTCAAGTTCAAGAAAAAGGAGA (SEQ ID NO: 29) |
| COL5A1 | NM_000093.3 | 6345-6445 | GTAAAGGTCATCCCACCATCACCAAAGCCTCCGTTTTTAA |
| | | | CAACCTCCAACACGATCCATTTAGAGGCCAAATGTCATTC |
| | | | TGCAGGTGCCTTCCCGATGG (SEQ ID NO: 30) |
| COL5A2 | NM_000393.3 | 4075-4175 | GGTTCATGCTACCCTGAAGTCACTCAGTAGTCAGATTGAA |
| | | | ACCATGCGCAGCCCCGATGGCTCGAAAAAGCACCCAGC |
| | | | CCGCACGTGTGATGACCTAAAG (SEQ ID NO: 31) |
| CTHRC1 | NM_138455.2 | 685-785 | CTGTGGAAGGACTTTGTGAAGGAATTGGTGCTGGATTAG |
| | | | TGGATGTTGCTATCTGGGTTGGCACTTGTTCAGATTACCC |
| | | | AAAAGGAGATGCTTCTACTGG (SEQ ID NO: 32) |
| CXCL1 | NM_001511.1 | 445-545 | AGGCCCTGCCCTTATAGGAACAGAAGAGGAAAGAGAGAC |
| | | | ACAGCTGCAGAGGCCACCTGGATTGTGCCTAATGTGTTT |
| | | | GAGCATCGCTTAGGAGAAGTCT (SEQ ID NO: 33) |
| CXCL13 | NM_006419.2 | 0-100 | GAGAAGATGTTTGAAAAAACTGACTCTGCTAATGAGCCTG |
| | | | GACTCAGAGCTCAAGTCTGAACTCTACCTCCAGACAGAA |
| | | | TGAAGTTCATCTCGACATCTC (SEQ ID NO: 34) |
| MMP1 | NM_002421.3 | 1117-1217 | AAATGGGCTTGAAGCTGCTTACGAATTTGCCGACAGAGA |
| | | | TGAAGTCCGGTTTTTCAAAGGGAATAAGTACTGGGCTGTT |
| | | | CAGGGACAGAATGTGCTACAC (SEQ ID NO: 35) |
| P4HA2 | NM_001017974.1 | 1600-1700 | TGTGCTTGTGGGCTGCAAGTGGGTCTCCAATAAGTGGTT |
| | | | CCATGAACGAGGACAGGAGTTCTTGAGACCTTGTGGATC |
| | | | AACAGAAGTTGACTGACATCCT (SEQ ID NO: 36) |
| PDPN | NM_006474.4 | 431-531 | CTCCAGGAACCAGCGAAGACCGCTATAAGTCTGGCTTGA |
| | | | CAACTCTGGTGGCAACAAGTGTCAACAGTGTAACAGGCA |
| | | | TTCGCATCGAGGATCTGCCAAC (SEQ ID NO: 37) |
| PLOD2 | NM_182943.2 | 2590-2690 | AAACATTGCACTTAATAACGTGGGAGAAGACTTTCAGGG |
| | | | AGGTGGTTGCAAATTTCTAAGGTACAATTGCTCTATTGAG |
| | | | TCACCACGAAAAGGCTGGAGC (SEQ ID NO: 38) |
| POSTN | NM_001135935.1 | 910-1010 | AGAGACGGTCACTTCACACTCTTTGCTCCCACCAATGAG |
| | | | GCTTTTGAGAAACTTCCACGAGGTGTCCTAGAAAGGATC |
| | | | ATGGGAGACAAAGTGGCTTCCG (SEQ ID NO: 39) |
| SDHA | NM_004168.1 | 230-330 | TGGAGGGGCAGGCTTGCGAGCTGCATTTGGCCTTTCTGA |
| | | | GGCAGGGTTTAATACAGCATGTGTTACCAAGCTGTTTCCT |
| | | | ACCAGGTCACACACTGTTGCA (SEQ ID NO: 40) |
| SERPIN | NM_000602.2 | 2470-2570 | TGTGTTCAATAGATTTAGGAGCAGAAATGCAAGGGGCTG |
| E1 | | | CATGACCTACCAGGACAGAACTTTCCCCAATTACAGGGT |
| | | | GACTCACAGCCGCATTGGTGAC (SEQ ID NO: 41) |
| SERPIN | NM_006216.2 | 240-340 | CGCTGCCTTCCATCTGCTCCCACTTCAATCCTCTGTCTCT |
| E2 | | | CGAGGAACTAGGCTCCAACACGGGGATCCAGGTTTTCAA |
| | | | TCAGATTGTGAAGTCGAGGCC (SEQ ID NO: 42) |
| SERPIN | NM_001235.2 | 880-980 | ATGGTGGACAACCGTGGCTTCATGGTGACTCGGTCCTAT |
| H1 | | | ACCGTGGGTGTCATGATGATGCACCGGACAGGCCTCTAC |
| | | | AACTACTACGACGACGAGAAGG (SEQ ID NO: 43) |
| THBS2 | NM_003247.2 | 4460-4560 | AAACATCCTTGCAAATGGGTGTGACGCGGTTCCAGATGT |
| | | | GGATTTGGCAAAACCTCATTTAAGTAAAAGGTTAGCAGAG |
| | | | CAAAGTGCGGTGCTTTAGCTG (SEQ ID NO: 44) |
| TNC | NM_002160.1 | 6885-6985 | CAGAAATCTTGAAGGCAGGCGCAAACGGGCATAAATTGG |
| | | | AGGGACCACTGGGTGAGAGAGGAATAAGGCGGCCCAGA |
| | | | GCGAGGAAAGGATTTTACCAAAG (SEQ ID NO: 45) |
| GAPDH | NM_002046.3 | 35-135 | TCCTCCTGTTCGACAGTCAGCCGCATCTTCTTTTGCGTCG |
| | | | CCAGCCGAGCCACATCGCTCAGACACCATGGGGAAGGT |
| | | | GAAGGTCGGAGTCAACGGATTT (SEQ ID NO: 46) |
| RPS18 | NM_022551.2 | 110-210 | GCGGCGGAAAATAGCCTTTGCCATCACTGCCATTAAGGG |
| | | | TGTGGGCCGAAGATATGCTCATGTGGTGTTGAGGAAAGC |
| | | | AGACATTGACCTCACCAAGAGG (SEQ ID NO: 47) |

GAPDH and RPS18 were used as internal controls for normalization of Nanostring data.

TABLE 12
Primer sequences used in the RQ-PCR experiments

| Gene | | Amplicon |
|---|---|---|
| symbol | Primer sequence | length |
| GAPDH | Forward 5′-CCTGTTCGACAGTCAGCCGCAT-3′ (SEQ ID NO: 48) | 87 bp |
| | Reverse 5′-GACTCCGACCTTCACCTTCCCC-3′ (SEQ ID NO: 49) | |
| RPS18 | Forward 5′-GCGGCGGAAAATAGCCTTTGCC-3′ (SEQ ID NO: 50) | 100 bp |
| | Reverse 5′-CCTCTTGGTGAGGTCAATGTCTGC-3′ (SEQ ID NO: 51) | |
| MMP1 | Forward 5′-CAAATGGGCTTGAAGCTGCTTACG-3′ (SEQ ID NO: 52) | 101 bp |
| | Reverse 5′-GTGTAGCACATTCTGTCCCTGAACA-3′ (SEQ ID NO: 53) | |
| COL4A1 | Forward 5′-AAGGACCAAAAGGTGACAAGGGTGA-3′ (SEQ ID NO: 54) | 72 bp |
| | Reverse 5′-GAACTTGAGCTTGTCCTGGTACTCC-3′ (SEQ ID NO: 55) | |
| COL5A1 | Forward 5′-GTCATCCCACCATCACCAAAGCC-3′ (SEQ ID NO: 56) | 92 bp |
| | Reverse 5′-ATCGGGAAGGCACCTGCAGAATG-3′ (SEQ ID NO: 57) | |
| THBS2 | Forward 5′-TTGCAAATGGGTGTGACGCGGT-3′ (SEQ ID NO: 58) | 86 bp |
| | Reverse 5′-AAGCACCGCACTTTGCTCTGCT-3′ (SEQ ID NO: 59) | |
| TNC | Forward 5′-ACGAACACTCAATCCAGTTTGCTGA-3′ (SEQ ID NO: 60) | 89 bp |
| | Reverse 5′-TGGAATTTATGCCCGTTTGCGCC-3′ (SEQ ID NO: 61) | |
| COL3A1 | Forward 5′-TGGCACAACAGGAAGCTGTTGAAGG-3′ (SEQ ID NO: 62) | 97 bp |
| | Reverse 5′-ACACATATTTGGCATGGTTCTGGCT-3′ (SEQ ID NO: 63) | |
| COL5A2 | Forward 5′-TCATGCTACCCTGAAGTCACTCAGT-3′ (SEQ ID NO: 64) | 93 bp |
| | Reverse 5′-AGGTCATCACACGTGCGGGC-3′ (SEQ ID NO: 65) | |
| PDPN | Forward 5′-CAGGAACCAGCGAAGACCGCT-3′ (SEQ ID NO: 66) | 95 bp |
| | Reverse 5′-TGGCAGATCCTCGATGCGAATGC-3′ (SEQ ID NO: 67) | |
| POSTN | Forward 5′-CGGTCACTTCACACTCTTTGCTCCC-3′ (SEQ ID NO: 68) | 95 bp |
| | Reverse 5′-CGGAAGCCACTTTGTCTCCCATGA-3′ (SEQ ID NO: 69) | |
| SERPINE2 | Forward 5′-ACCATGAACTGGCATCTCCCCCT-3′ (SEQ ID NO: 70) | 100 bp |
| | Reverse 5′-TGGAGCCTAGTTCCTCGAGAGACA-3′ (SEQ ID NO: 71) | |
| SERPINH1 | Forward 5′-CCGTGGCTTCATGGTGACTCGG-3′ (SEQ ID NO: 72) | 74 bp |
| | Reverse 5′-AGTAGTTGTAGAGGCCTGTCCGGT-3′ (SEQ ID NO: 73) | |
| SDHA | Forward 5′-CTCCAAGCCCATCCAGGGGCAA-3′ (SEQ ID NO: 74) | 100 bp |
| | Reverse 5′-CAGAGTGACCTTCCCAGTGCCAA-3′ (SEQ ID NO: 75) | |
| PLOD2 | Forward 5′-TGGCTCTTTGCCGAAATGCTAGAG-3′ (SEQ ID NO: 76) | 87 bp |
| | Reverse 5′-GGGGGCTGAGCATTTGGAATGTTT-3′ (SEQ ID NO: 77) | |
| P4HA2 | Forward: 5′-AGGAGCTGCCAAAGCCCTGA-3′ (SEQ ID NO: 78) | 170 bp |
| | Reverse: 5′-ACCTGCTCCATCCACAACACCG-3′ (SEQ ID NO: 79) | |
| CTHRC1 | Forward: 5′-TTGTTCAGTGGCTCACTTCG-3′ (SEQ ID NO: 80) | 102 bp |
| | Reverse: 5′-TTCAATGGGAAGAGGTCCTG-3′ (SEQ ID NO: 81) | |
| CXCL1 | Forward: 5′-ATTTCTGAGGAGCCTGCAAC-3′ (SEQ ID NO: 82) | 100 bp |
| | Reverse: 5′-CACATACATTCCCCTGCCTT-3′ (SEQ ID NO: 83) | |
| CXCL13 | Forward: 5′-GAGCCTGTCAAGAGGCAAAG-3′ (SEQ ID NO: 84) | 142 bp |
| | Reverse: 5′-CTGGGGATCTTCGAATGCTA-3′ (SEQ ID NO: 85) | |

SERPINE1 was excluded from RQ-PCR analysis since no primer pairs tested showed good efficiency for amplification in FFPE samples.
Primer sequences used yielded short amplicon lengths, as indicated.

### Example 7

### Relationship Between Hazard of Recurrence and Over-Expression of the Four-Gene Signature in Histologically Normal Margins:

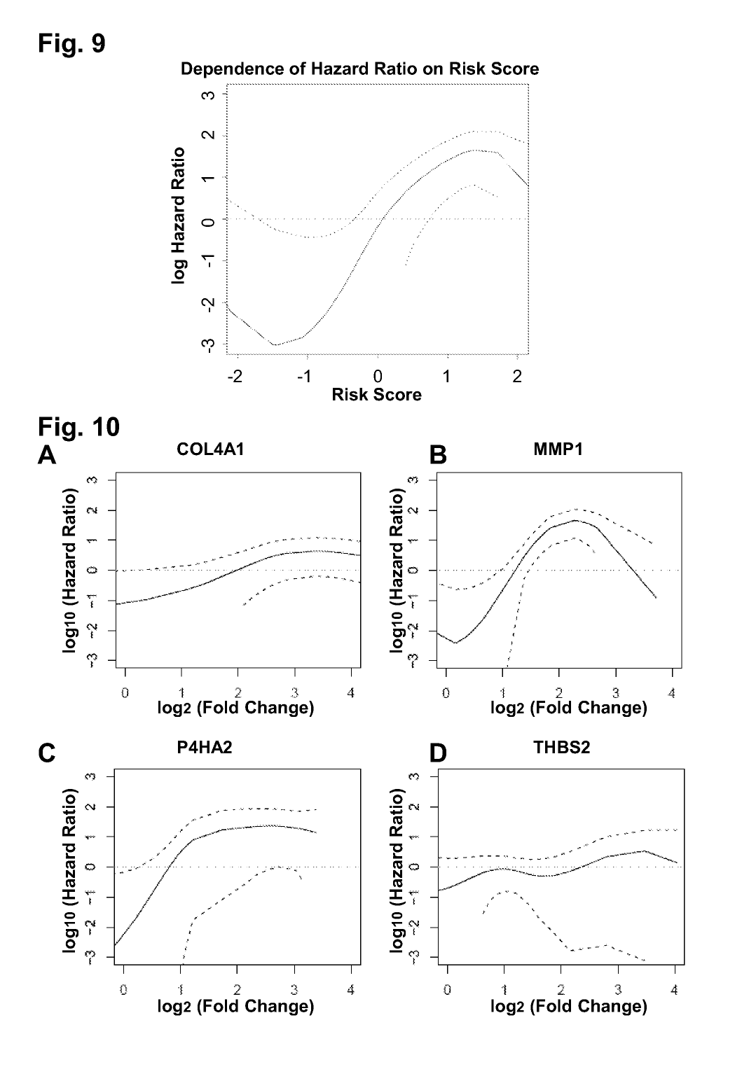

A sensitivity analysis using the quantitative PCR data is given in FIGS. 9 and q0. This analysis shows the relationship between hazard of recurrence and over-expression of each gene. The dashed lines give an 80% confidence interval, which is wide because of the small sample size. The strength of association is different for each gene, being strongest for P4HA2 and MMP1. For P4HA2 and MMP1, a 50% increase in expression could confer a substantial increased risk of recurrence (˜5-fold), and for COL4A1 and THBS2 a 2-fold increase produces a comparable increase in risk.

### Sample Testing:

The sample being tested would typically be compared to a standard normal sample, for example tongue tissue from healthy individuals, or a value corresponding thereto. For optimal reproducibility, a universal RNA pool would be used as the reference RNA sample for PCR. In this case the margin sample would be compared to a predetermined range established for example from a larger clinical trial. The kit would contain reference RNA, PCR primers for the four-gene signature plus housekeeping genes, and the pre-determined recurrence of risk associated with different values of the risk score.

### Risk Score Calculation:

The relative expression of each gene in the four-gene signature will be calculated from quantitative PCR−Ct (Cycle threshold) values. Ct values are used in an algorithm—the delta delta Ct method (69) to determine relative gene expression. These values will be used to calculate the combined risk score by a weighted average, with weights given in table (n) (e.g calculatable from a large clinical trial). These values of the risk score will be used in conjunction with a pre-established table to look up risk of recurrence based on the patents' score. In the current analysis, patients are considered “high risk” if their risk score is above the median risk score determined from the training set (score=0.2), and “low risk” if their score is below this threshold. In this example, “high risk” patients in the validation set are 7 times more likely to experience recurrence (95% Cl=0.8−58, Wald Test) than “low risk” patients. A more detailed risk table will be determined by a clinical trial with larger sample size than the current validation set (which has n=30 patients).

### Protein Expression Analysis as a Predictor of Recurrence:

As mRNA levels and protein levels correlate in the majority of cases, antibody-based methods for detection of proteins would also work for predicting the risk of recurrence. In this method, immunohistochemical analysis using specific antibodies would be used to detect the presence of gene products of the four genes in the signature. In this case, qualitative (or semi-quantitative) scoring rules for one or more of the genes in the four-gene signature could be developed based on the larger validation set where these methods would be applied to surgical resection margins of oral carcinoma.

Recent studies in the literature have shown antibody-based work for protein detection of MMP1, P4HA2, as described below.

A recent study showed higher protein expression (as detected by Immunohistochemistry) of several matrix metalloproteinases (including MMP1) in oral tongue and lip tissue (67).

In another recent publication, higher levels of P4HA2 protein were detected by Immunohistochemistry and associated with metastasis of oral carcinoma (68).

Therefore, antibodies for proteins encoded by genes in the prognostic signature may be available and optimized for use in surgical resection margins.

Thus far, a publicly available database (The Human Protein Atlas; http://www.proteinatlas.orq/) contains validation data by Immunohistochemistry on the following antibodies (included in the four-gene prognostic signature):
THBS2 Protein: Antibody ID CAB017716—antibody intensity of staining varies from weak to moderate in different tissue samples. This antibody shows weak intensity of staining in oral mucosa tissue samples (information and illustrations of data available at the Human Protein Atlas website at http://www.proteinatlas.orq/ENSG00000186340/normal/oral+mucosa).
COL4A1 Protein: Antibody ID CAB001695—antibody intensity of staining varies from weak to moderate in different tissue samples. This antibody shows negative expression of COL4A1 in oral mucosa tissue (information and illustrations of data available at the Human Protein Atlas website at http://vvww.proteinatlas.org/ENSG00000187498/normal/oral+mucosa).

While the present disclosure has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. All sequences (e.g., nucleotide, including RNA and cDNA, and polypeptide sequences) of genes listed in Table 3, Table 4, Table 5, Table 7, Table 9, Table 10 and Table 12 for example referred to by accession number are herein incorporated specifically by reference.

### REFERENCES

1. Parkin D M, Pisani P, Ferlay J. Global cancer statistics. CA Cancer J. Clin. 1999 January-February; 49(1):33-64, 1.
2. Sawair F A, Irwin C R, Gordon D J, Leonard A G, Stephenson M, Napier S S. Invasive front grading: reliability and usefulness in the management of oral squamous cell carcinoma. J Oral Pathol Med. 2003 January; 32(1):1-9.
3. Jones A S, Bin Hanafi Z, Nadapalan V, Roland N J, Kinsella A, Helliwell T R. Do positive resection margins after ablative surgery for head and neck cancer adversely affect prognosis? A study of 352 patients with recurrent carcinoma following radiotherapy treated by salvage surgery. Br J. Cancer. 1996 July; 74(1):128-32.
4. Leemans C R, Tiwari R, Nauta J J, van der Waal I, Snow G B. Recurrence at the primary site in head and neck cancer and the significance of neck lymph node metastases as a prognostic factor. Cancer. 1994 Jan. 1; 73(1):187-90.
5. Brandwein-Gensler M, Teixeira M S, Lewis C M, Lee B, RoInitzky L, Hille J J, et al. Oral squamous cell carcinoma: histologic risk assessment, but not margin status, is strongly predictive of local disease-free and overall survival. Am J Surg Pathol. 2005 February; 29(2):167-78.
6. Nathan C A, Amirghahri N, Rice C, Abreo F W, Shi R, Stucker F J. Molecular analysis of surgical margins in head and neck squamous cell carcinoma patients. Laryngoscope. 2002 December; 112(12):2129-40.
7. Bilde A, von Buchwald C, Dabelsteen E, Therkildsen M H, Dabelsteen S. Molecular markers in the surgical margin of oral carcinomas. J Oral Pathol Med. 2009 January; 38(1):72-8.
8. Nathan C A, Liu L, Li B D, Abreo F W, Nandy I, De Benedetti A. Detection of the proto-oncogene elF4E in surgical margins may predict recurrence in head and neck cancer. Oncogene. 1997 Jul. 31; 15(5):579-84.
9. Nathan C A, Franklin S, Abreo F W, Nassar R, De Benedetti A, Glass J. Analysis of surgical margins with the molecular marker elF4E: a prognostic factor in patients with head and neck cancer. J Clin Oncol. 1999 September; 17(9):2909-14.
10. Tan H K, Saulnier P, Auperin A, Lacroix L, Casiraghi O, Janot F, et al. Quantitative methylation analyses of resection margins predict local recurrences and disease-specific deaths in patients with head and neck squamous cell carcinomas. Br J. Cancer. 2008 Jul. 22; 99(2):357-63.
11. van der Toorn P P, Veltman J A, Bot F J, de Jong J M, Manni J J, Ramaekers F C, et al. Mapping of resection margins of oral cancer for p53 overexpression and chromosome instability to detect residual (pre)malignant cells. J. Pathol. 2001 January; 193(1):66-72.
12. van Houten V M, Leemans C R, Kummer J A, Dijkstra J, Kuik D J, van den Brekel M W, et al. Molecular diagnosis of surgical margins and local recurrence in head and neck cancer patients: a prospective study. Clin Cancer Res. 2004 Jun. 1; 10(11):3614-20.
13. Goldenberg D, Harden S, Masayesva B G, Ha P, Benoit N, Westra W H, et al. Intraoperative molecular margin analysis in head and neck cancer. Arch Otolaryngol Head Neck Surg. 2004 January; 130(1):39-44.
14. Franklin S, Pho T, Abreo F W, Nassar R, De Benedetti A, Stucker F J, et al. Detection of the proto-oncogene elF4E in larynx and hypopharynx cancers. Arch Otolaryngol Head Neck Surg. 1999 February; 125(2): 177-82.
15. Taioli E, Ragin C, Wang X H, Chen J, Langevin S M, Brown A R, et al. Recurrence in oral and pharyngeal cancer is associated with quantitative MGMT promoter methylation. BMC Cancer. 2009; 9:354.
16. van Houten V M, Tabor M P, van den Brekel M W, Kummer J A, Denkers F, Dijkstra J, et al. Mutated p53 as a molecular marker for the diagnosis of head and neck cancer. J. Pathol. 2002 December; 198(4):476-86.
17. R development core team. R: A Language and Environment for Statistical Computing Vienna, Austria; 2009.
18. Gentleman R. Bioinformatics and computational biology solutions using R and Bioconductor 2005 [cited; 1st ed.: [Available from: http://www.worldcat.orq/isbn/0387251464]
19. McCarthy D J, Smyth G K. Testing significance relative to a fold-change threshold is a TREAT. Bioinformatics. 2009 Mar. 15; 25(6):765-71.
20. Goeman J J. Penalized estimation in the Cox proportional hazards model. Biometrical journal Biometrische Zeitschrift. 2010 February; 52(1):70-84.
21. Tabor M P, Brakenhoff R H, van Houten V M, Kummer J A, Snel M H, Snijders P J, et al. Persistence of genetically altered fields in head and neck cancer patients: biological and clinical implications. Clin Cancer Res. 2001 June; 7(6):1523-32.
22. Ha P K, Califano J A. The molecular biology of mucosal field cancerization of the head and neck. Crit. Rev Oral Biol Med. 2003; 14(5):363-9.
23. Jarzab B, Wiench M, Fujarewicz. K, Simek K, Jarzab M, Oczko-Wojciechowska M, et al. Gene expression profile of papillary thyroid cancer: sources of variability and diagnostic implications. Cancer Res. 2005 Feb. 15; 65(4):1587-97.
24. Bornstein P, Kyriakides T R, Yang Z, Armstrong L C, Birk D E. Thrombospondin 2 modulates collagen fibrillogenesis and angiogenesis. J Investig Dermatol Symp Proc. 2000 December; 5(1):61-6.
25. Sado Y, Kagawa M, Naito I, Ueki Y, Seki T, Momota R, et al. Organization and expression of basement membrane collagen IV genes and their roles in human disorders. J. Biochem. 1998 May; 123(5):767-76.
26. Hoffmann R, Valencia A. A gene network for navigating the literature. Nat. Genet. 2004 July; 36(7):664.
27. Tanzer M L. Current concepts of extracellular matrix. J Orthop Sci. 2006 May; 11(3):326-31.
28. Chen C, Mendez E, Houck J, Fan W, Lohavanichbutr P, Doody D, et al. Gene expression profiling identifies genes predictive of oral squamous cell carcinoma. Cancer Epidemiol Biomarkers Prey. 2008 August; 17(8):2152-62.
29. Egeblad M, Werb Z. New functions for the matrix metalloproteinases in cancer progression. Nat Rev Cancer. 2002 March; 2(3):161-74.
30. Ginos M A, Page G P, Michalowicz B S, Patel K J, Volker S E, Pambuccian S E, et al. Identification of a gene expression signature associated with recurrent disease in squamous cell carcinoma of the head and neck. Cancer Res. 2004 Jan. 1; 64(1):55-63.
31. Reis P P, Rogatto S R, Kowalski L P, Nishimoto I N, Montovani J C, Corpus G, et al. Quantitative real-time PCR identifies a critical region of deletion on 22q13 related to prognosis in oral cancer. Oncogene. 2002 Sep. 19; 21(42):6480-7.
32. Reis P P B, R R.; Machado, J.; MacMillan, C.; Pintilie, M.; Sukhai, M A.; Perez-Ordonez, B.; Gullane, P.; Irish, J.; Kamel-Reid, S. Claudin 1 over-expression increases invasion and is associated with aggressive histological features in oral squamous cell carcinoma. Cancer. 2008.
33. Livak K J, Schmittgen T D. Analysis of relative gene expression data using real-time quantitative PCR and the 2(-Delta Delta C(T)) Method. Methods. 2001 December; 25(4):402-8.
34. Toruner G A, Ulger C, Alkan M, Galante A T, Rinaggio J, Wilk R, et al. Association between gene expression profile and tumor invasion in oral squamous cell carcinoma. Cancer Genet Cytogenet. 2004 Oct. 1; 154(1):27-35.
35. Ye H, Yu T, Temam S, Ziober B L, Wang J, Schwartz J L, et al. Transcriptomic dissection of tongue squamous cell carcinoma. BMC Genomics. 2008; 9:69.
36. Kuriakose M A, Chen W T, He Z M, Sikora A G, Zhang P, Zhang Z Y, et al. Selection and validation of differentially expressed genes in head and neck cancer. Cell Mol Life Sci. 2004 June; 61(11):1372-83.
37. Sticht C, Freier K, Knopfle K, Flechtenmacher C, Pungs S, Hofele C, et al. Activation of MAP kinase signaling through ERK5 but not ERK1 expression is associated with lymph node metastases in oral squamous cell carcinoma (OSCC). Neoplasia. 2008 May; 10(5)1462-70.
38. Pyeon D, Newton M A, Lambert P F, den Boon J A, Sengupta S, Marsit C J, et al. Fundamental differences in cell cycle deregulation in human papillomavirus-positive and human papillomavirus-negative head/neck and cervical cancers. Cancer Res. 2007 May 15; 67(10):4605-19.
39. Wu Z, Irizarry, R. A., Gentleman, R., Martinez-Murillo, F., Spencer, F. A Model-Based Background Adjustment for Oligonucleotide Expression Arrays. Journal of the American Statistical Association. 2004; 99(468):909-17.
40. Dai M, Wang P, Boyd A D, Kostov G, Athey B, Jones E G, et al. Evolving gene/transcript definitions significantly alter the interpretation of GeneChip data. Nucleic Acids Res. 2005; 33(20):e175.
41. Gautier L, Cope L, Bolstad B M, Irizarry R A. affy—analysis of Affymetrix GeneChip data at the probe level. Bioinformatics. 2004 Feb. 12; 20(3):307-15.
42. Hong F, Breitling R, McEntee C W, Wittner B S, Nemhauser J L, Chory J. RankProd: a bioconductor package for detecting differentially expressed genes in meta-analysis. Bioinformatics. 2006 Nov. 15; 22(22):2825-7.
43. Falcon S, Gentleman R. Using GOstats to test gene lists for GO term association. Bioinformatics. 2007 Jan. 15; 23(2):257-8.
44. Zheng Q, Wang X J. GOEAST: a web-based software toolkit for Gene Ontology enrichment analysis. Nucleic Acids Res. 2008 Jul. 1; 36(Web Server issue):W358-63.
45. Brown K R, Jurisica I. Online predicted human interaction database. Bioinformatics. 2005 May 1; 21(9):2076-82.
46. Brown K R, Otasek D, Ali M, McGuffin M J, Xie W, Devani B, et al. NAViGaTOR: Network Analysis, Visualization and Graphing Toronto. Bioinformatics. 2009 Dec. 15; 25(24):3327-9.
47. McGuffin M J, Jurisica I. Interaction techniques for selecting and manipulating subgraphs in network visualizations. IEEE Trans Vis Comput Graph. 2009 November-December; 15(6):937-44.
48. Carmona-Saez P, Chagoyen M, Tirado F, Carazo J M, Pascual-Montano A. GENECODIS: a web-based tool for finding significant concurrent annotations in gene lists. Genome Biol. 2007; 8(1):R3.
49. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 2005 Oct. 25; 102(43):15545-50.
50. Cheong S C et al. Gene expression in human oral squamous cell carcinoma is influenced by risk factor exposure J Oral Oncology. 2009; 45: 712-719.
51. von Ahlfen S, Missel A, Bendrat K, Schlumpberger M: Determinants of RNA quality from FFPE samples. PLoS One 2007, 2(12):e1261.
52. Masuda N, Ohnishi T, Kawamoto S, Monden M, Okubo K: Analysis of chemical modification of RNA from formalin-fixed samples and optimization of molecular biology applications for such samples. Nucleic Acids Res 1999, 27(22):4436-4443.
53. Bresters D, Schipper M E, Reesink H W, Boeser-Nunnink B D, Cuypers H T: The duration of fixation influences the yield of HCV cDNA-PCR products from formalin-fixed, paraffin-embedded liver tissue. J Virol Methods 1994, 48(2-3):267-272.
54. Macabeo-Ong M, Ginzinger D G, Dekker N, McMillan A, Regezi J A, Wong D T, Jordan R C: Effect of duration of fixation on quantitative reverse transcription polymerase chain reaction analyses. Mod Pathol 2002, 15(9):979-987.
55. Geiss G K, Bumgarner R E, Birditt B, Dahl T, Dowidar N, Dunaway D L, Fell H P, Ferree S, George R D, Grogan T et al: Direct multiplexed measurement of gene expression with color-coded probe pairs. Nat Biotechnol 2008, 26(3):317-325.
56. Reis P P, Bharadwaj R R, Machado J, Macmillan C, Pintilie M, Sukhai M A, Perez-Ordonez B, Gullane P, Irish J, Kamel-Reid S: Claudin 1 overexpression increases invasion and is associated with aggressive histological features in oral squamous cell carcinoma. Cancer 2008, 113(11):3169-3180.
57. Cervigne N K, Reis P P, Machado J, Sadikovic B, Bradley G, Galloni N N, Pintilie M, Jurisica I, Perez-Ordonez B, Gilbert R et al: Identification of a microRNA signature associated with progression of leukoplakia to oral carcinoma. Hum Mol Genet. 2009, 18(24):4818-4829.
58. Dos Reis P P, Bharadwaj R R, Machado J, Macmillan C, Pintilie M, Sukhai M A, Perez-Ordonez B, Gullane P, Irish J, Kamel-Reid S: Claudin 1 overexpression increases invasion and is associated with aggressive histological features in oral squamous cell carcinoma. Cancer 2008, 113(11):3169-3180.
59. Reis P P, Tomenson M, Cervigne N K, Machado J, Jurisica I, Pintilie M, Sukhai M A, Perez-Ordonez B, Grenman R, Gilbert R W et al: Programmed cell death 4 loss increases tumor cell invasion and is regulated by miR-21 in oral squamous cell carcinoma. Mol Cancer 2010, 9:238.
60. Livak K J, Schmittgen T D: Analysis of relative gene expression data using real-time quantitative PCR and the 2(-Delta Delta C(T)) Method. Methods 2001, 25(4):402-408.
61. Rodgers J L, Nicewander, W. A.: Thirteen ways to look at the correlation coefficient. The American Statistician 1988, 42(1):59-66.
62. R Development Core Team: R: A Language and Environment for Statistical Computing. Vienna, Austria; 2008.
63. Sanchez-Navarro I, Gamez-Pozo A, Gonzalez-Baron M, Pinto-Marin A, Hardisson D, Lopez R, Madero R, Cejas P, Mendiola M, Espinosa E et al: Comparison of gene expression profiling by reverse transcription quantitative PCR between fresh frozen and formalin-fixed, paraffin-embedded breast cancer tissues. Biotechniques 2010, 48(5):389-397.
64. Cronin M, Pho M, Dutta D, Stephans J C, Shak S, Kiefer M C, Esteban J M, Baker J B: Measurement of gene expression in archival paraffin-embedded tissues: development and performance of a 92-gene reverse transcriptase-polymerase chain reaction assay. Am J Pathol 2004, 164(1):35-42.
65. Paik S, Shak S, Tang G, Kim C, Baker J, Cronin M, Baehner F L, Walker M G, Watson D, Park T et al: A multigene assay to predict recurrence of tamoxifen-treated, node-negative breast cancer. N Engl J Med 2004, 351(27):2817-2826.
66. Antonov J, Goldstein D R, Oberli A, Baltzer A, Pirotta M, Fleischmann A, Alternatt H J, Jaggi R: Reliable gene expression measurements from degraded RNA by quantitative real-time PCR depend on short amplicons and a proper normalization. Lab Invest 2005, 85(8):1040-1050.
67. Barros S S, Henriques K C, Pereira K M, de Medeiros A M, Galva® H C, Freitas R A. Immunohistochemical expression of matrix metalloproteinases in squamous cell carcinoma of the tongue and lower lip. Arch Oral Biol 2011 August; 56(8):752-60.
68. Chang K P, Yu J S, Chien K Y, Lee C W, Liang Y, Liao C T, Yen T C, Lee L Y, Huang L L, Liu S C, Chang Y S, Chi L M. Identification of PRDX4 and P4HA2 as metastasis-associated proteins in oral cavity squamous cell carcinoma by comparative tissue proteomics of microdissected specimens using iTRAQ technology. J Proteome Res. 2011. Nov 4; 10(11):4935-47.
69. Pfaffl M W. A new mathematical model for relative quantification in real-time RT-PCR. Nucleic Acids Res. 2001 May 1; 29(9):e45.
70. Chen C, Mendez E, Houck J, Fan W, Lohavanichbutr P, Doody D, Yueh B, Futran N D, Upton M, Farwell D G et al: Gene expression profiling identifies genes predictive of oral squamous cell carcinoma. Cancer Epidemiol Biomarkers Prev 2008, 17(8):2152-2162.
71. Yen C Y, Chen C H, Chang C H, Tseng H F, Liu S Y, Chuang L Y, Wen C H, Chang H W: Matrix metalloproteinases (MMP) 1 and MMP10 but not MMP12 are potential oral cancer markers. Biomarkers 2009, 14(4):244-249.
72. Geiss G K, Bumgarner R E, Birditt B, Dahl T, Dowidar N, Dunaway D L, Fell H P, Ferree S, George R D, Grogan T et al: Direct multiplexed measurement of gene expression with color-coded probe pairs. Nat Biotechnol 2008, 26(3):317-325.

1. (canceled)

2. A method of diagnosing or predicting a likelihood of OSCC recurrence in a subject comprising:
a) determining an expression level of one or more biomarkers selected from MMP1, COL4A1, THBS2 and P4HA2 in a test sample from the subject, the one or more biomarkers comprising at least one of THBS2 and P4HA2, and
b) comparing the expression level of the one or more biomarkers with a control,
diagnosing or predicting the likelihood of OSCC recurrence in the subject, based on a difference or a similarity in the expression level of the one or more biomarkers between the test sample and the control; wherein the one or more biomarkers does not consist of THBS2 and COL4A1.

3. The method of claim 2, wherein the one or more biomarkers comprise MMP1, COL4A1, THBS2 and P4HA2.

4. The method of claim 2, wherein the biomarkers further include at least one or both of PXDN or PMEPA1.

5. The method claim 2, wherein an increase in the expression of level of at least 1, at least 2, at least 3, at least 4 or more of the biomarkers compared to the control is indicative of an increased likelihood of recurrence of OSCC in the subject.

6. The method of claim 2, wherein the expression level of the one or more biomarkers is used to calculate a risk score for the subject, wherein the risk score calculation comprises summing a weighted expression level for each of the one or more biomarkers determined in the test sample.

7. The method of claim 6, wherein the weighted expression level comprises the relative expression level multiplied by a coefficient specific for the biomarker, optionally a coefficient in Table 6.

8. The method of claim 2, wherein the comparing the expression level of the one or more biomarkers in the test sample with a control comprises determining the relative expression of each biomarker, calculating a risk score for the subject, and using the risk score to classify the subject as having a high-risk of recurrence of OSCC or a low-risk of recurrence of OSCC by comparing the risk score to a control wherein the control is a threshold score associated with a population of subjects known to have OSCC without recurrence.

9. The method of claim 6, wherein the subject is predicted to have a high risk of recurrence when the risk score is greater than the control.

10. The method of claim 2, wherein the sample comprises a histologically normal surgical resection margin.

11. The method of claim 2, wherein the expression level determined is a nucleic acid expression level.

12. The method of claim 11, wherein determining the biomarker expression level comprises use of quantitative PCR, such as quantitative RT-PCR, serial analysis of gene expression (SAGE), microarray, digital molecular barcoding technology, such as Nanostring analysis or Northern Blot or other probe based or amplification based assay.

13. The method of claim 11, wherein determining the biomarker expression level comprises amplification of the nucleic acid expression level using a primer or primer set.

14. The method of claim 13, wherein the primer or primer set comprises a nucleic acid sequence selected from any one of SEQ ID NO:_{—}1 to 8, SEQ ID NO: 52 to 55, SEQ ID NO: 58 to 59 or SEQ ID NO: 78 to 79.

15. The method of claim 12, wherein determining the biomarker expression level comprises using an array and/or digital molecular barcoding technology.

16. The method of claim 15, wherein the probe comprises one or more of SEQ ID NO: 24 to 27, SEQ ID NO: 35, SEQ ID NO: 29, SEQ ID NO: 44 or SEQ ID NO: 36.

17. The method of claim 2, wherein the expression level determined is a polypeptide level.

18. The method of claim 17, wherein the biomarker expression level is determined using an antibody that specifically binds to the polypeptide and assaying the polypeptide level by optionally immunohistochemistry.

19. The method of claim 2, wherein the test sample comprises an oral tissue sample comprising histologically normal tumor resection margin tissue.

20. The method of claim 19, wherein the oral tissue sample comprises buccal mucosa, floor of the mouth (FOM), tongue, alveolar, palate, gingival or retromolar tissue.

21. A method of treating a subject in need thereof comprising:
a) obtaining a test sample from the subject;
b) predicting the likelihood of recurrence of OSCC in the subject according to the method of claim 2; and
c) administering to the subject predicted to have an increased likelihood of OSCC recurrence a treatment suitable for OSCC or a pre-OSCC condition.

22. The method of claim 21, wherein the treatment is adjuvant post-operative radiation treatment.

23.-38. (canceled)


SEQUENCE LISTING (85 sequences)

SEQ ID NO: 1 - DNA, 20 bp, Homo Sapien
tgctcatgcttttcaaccag

SEQ ID NO: 2 - DNA, 20 bp, Homo Sapien
ccgcaacacgatgtaagttg

SEQ ID NO: 3 - DNA, 20 bp, Homo Sapien
agcagaaggactgccggggt

SEQ ID NO: 4 - DNA, 20 bp, Homo Sapien
caatgcctggctggcccaca

SEQ ID NO: 5 - DNA, 20 bp, Homo Sapien
ggtcggcctgcactgtcacc

SEQ ID NO: 6 - DNA, 20 bp, Homo Sapien
ggggaagctgctgcactggg

SEQ ID NO: 7 - DNA, 20 bp, Homo Sapien
aggagctgccaaagccctga

SEQ ID NO: 8 - DNA, 22 bp, Homo Sapien
acctgctccatccacaacaccg

SEQ ID NO: 9 - DNA, 20 bp, Homo Sapien
ggcctccaaggagtaagacc

SEQ ID NO: 10 - DNA, 20 bp, Homo Sapien
aggggtctacatggcaactg

SEQ ID NO: 11 - DNA, 2081 bp, Homo Sapien
agcatgagtcagacagcctctggctttctggaagggcaaggactctatatatacagagggagcttcctagctgggatattggagcagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcagctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgttttatacactttcaaaaaaaaaaaaaaaaaaaaaaaaaa

SEQ ID NO: 12 - protein, 403 aa, Homo Sapien
MQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 13 - DNA, 6549 bp, Homo Sapien
gcttggagccgccgcacccgggacggtgcgtagcgctggaagtccggccttccgagagctagctgtccgccgcggcccccgcacgccgggcagccgtccctcgccgcctcgggcgcgccaccatggggccccggctcagcgtctggctgctgctgctgcccgccgcccttctgctccacgaggagcacagccgggccgctgcgaagggtggctgtgctggctctggctgtggcaaatgtgactgccatggagtgaagggacaaaagggtgaaagaggcctcccggggttacaaggtgtcattgggtttcctggaatgcaaggacctgaggggccacagggaccaccaggacaaaagggtgatactggagaaccaggactacctggaacaaaagggacaagaggacctccgggagcatctggctaccctggaaacccaggacttcccggaattcctggccaagacggcccgccaggccccccaggtattccaggatgcaatggcacaaagggggagagagggccgctcgggcctcctggcttgcctggtttcgctggaaatcccggaccaccaggcttaccagggatgaagggtgatccaggtgagatacttggccatgtgcccgggatgctgttgaaaggtgaaagaggatttcccggaatcccagggactccaggcccaccaggactgccagggcttcaaggtcctgttgggcctccaggatttaccggaccaccaggtcccccaggccctcccggccctccaggtgaaaagggacaaatgggcttaagttttcaaggaccaaaaggtgacaagggtgaccaaggggtcagtgggcctccaggagtaccaggacaagctcaagttcaagaaaaaggagacttcgccaccaagggagaaaagggccaaaaaggtgaacctggatttcaggggatgccaggggtcggagagaaaggtgaacccggaaaaccaggacccagaggcaaacccggaaaagatggtgacaaaggggaaaaagggagtcccggttttcctggtgaacccgggtacccaggactcataggccgccagggcccgcagggagaaaagggtgaagcaggtcctcctggcccacctggaattgttataggcacaggacctttgggagaaaaaggagagaggggctaccctggaactccggggccaagaggagagccaggcccaaaaggtttcccaggactaccaggccaacccggacctccaggcctccctgtacctgggcaggctggtgcccctggcttccctggtgaaagaggagaaaaaggtgaccgaggatttcctggtacatctctgccaggaccaagtggaagagatgggctcccgggtcctcctggttcccctgggccccctgggcagcctggctacacaaatggaattgtggaatgtcagcccggacctccaggtgaccagggtcctcctggaattccagggcagccaggatttataggcgaaattggagagaaaggtcaaaaaggagagagttgcctcatctgtgatatagacggatatcgggggcctcccgggccacagggacccccgggagaaataggtttcccagggcagccaggggccaagggcgacagaggtttgcctggcagagatggtgttgcaggagtgccaggccctcaaggtacaccagggctgataggccagccaggagccaagggggagcctggtgagttttatttcgacttgcggctcaaaggtgacaaaggagacccaggctttccaggacagcccggcatgacagggagagcgggttctcctggaagagatggccatccgggtcttcctggccccaagggctcgccgggttctgtaggattgaaaggagagcgtggcccccctggaggagttggattcccaggcagtcgtggtgacaccggcccccctgggcctccaggatatggtcctgctggtcccattggtgacaaaggacaagcaggctttcctggaggccctggatccccaggcctgccaggtccaaagggtgaaccaggaaaaattgttcctttaccaggcccccctggagcagaaggactgccggggtccccaggcttcccaggtccccaaggagaccgaggctttcccggaaccccaggaaggccaggcctgccaggagagaagggcgctgtgggccagccaggcattggatttccagggccccccggccccaaaggtgttgacggcttacctggagacatggggccaccggggactccaggtcgcccgggatttaatggcttacctgggaacccaggtgtgcagggccagaagggagagcctggagttggtctaccgggactcaaaggtttgccaggtcttcccggcattcctggcacacccggggagaaggggagcattggggtaccaggcgttcctggagaacatggagcgatcggaccccctgggcttcaggggatcagaggtgaaccgggacctcctggattgccaggctccgtggggtctccaggagttccaggaataggcccccctggagctaggggtccccctggaggacagggaccaccggggttgtcaggccctcctggaataaaaggagagaagggtttccccggattccctggactggacatgccgggccctaaaggagataaaggggctcaaggactccctggcataacgggacagtcggggctccctggccttcctggacagcagggggctcctgggattcctgggtttccaggttccaagggagaaatgggcgtcatggggacccccgggcagccgggctcaccaggaccagtgggtgctcctggattaccgggtgaaaaaggggaccatggctttccgggctcctcaggacccaggggagaccctggcttgaaaggtgataagggggatgtcggtctccctggcaagcctggctccatggataaggtggacatgggcagcatgaagggccagaaaggagaccaaggagagaaaggacaaattggaccaattggtgagaagggatcccgaggagaccctgggaccccaggagtgcctggaaaggacgggcaggcaggacagcctgggcagccaggacctaaaggtgatccaggtataagtggaaccccaggtgctccaggacttccgggaccaaaaggatctgttggtggaatgggcttgccaggaacacctggagagaaaggtgtgcctggcatccctggcccacaaggttcacctggcttacctggagacaaaggtgcaaaaggagagaaagggcaggcaggcccacctggcataggcatcccagggctgcgaggtgaaaagggagatcaagggatagcgggtttcccaggaagccctggagagaagggagaaaaaggaagcattgggatcccaggaatgccagggtccccaggccttaaagggtctcccgggagtgttggctatccaggaagtcctgggctacctggagaaaaaggtgacaaaggcctcccaggattggatggcatccctggtgtcaaaggagaagcaggtcttcctgggactcctggccccacaggcccagctggccagaaaggggagccaggcagtgatggaatcccggggtcagcaggagagaagggtgaaccaggtctaccaggaagaggattcccagggtttccaggggccaaaggagacaaaggttcaaagggtgaggtgggtttcccaggattagccgggagcccaggaattcctggatccaaaggagagcaaggattcatgggtcctccggggccccagggacagccggggttaccgggatccccaggccatgccacggaggggcccaaaggagaccgcggacctcagggccagcctggcctgccaggacttccgggacccatggggcctccagggcttcctgggattgatggagttaaaggtgacaaaggaaatccaggctggccaggagcacccggtgtcccagggcccaagggagaccctggattccagggcatgcctggtattggtggctctccaggaatcacaggctctaagggtgatatggggcctccaggagttccaggatttcaaggtccaaaaggtcttcctggcctccagggaattaaaggtgatcaaggcgatcaaggcgtcccgggagctaaaggtctcccgggtcctcctggccccccaggtccttacgacatcatcaaaggggagcccgggctccctggtcctgagggccccccagggctgaaagggcttcagggactgccaggcccgaaaggccagcaaggtgttacaggattggtgggtatacctggacctccaggtattcctgggtttgacggtgcccctggccagaaaggagagatgggacctgccgggcctactggtccaagaggatttccaggtccaccaggccccgatgggttgccaggatccatggggcccccaggcaccccatctgttgatcacggcttccttgtgaccaggcatagtcaaacaatagatgacccacagtgtccttctgggaccaaaattctttaccacgggtactctttgctctacgtgcaaggcaatgaacgggcccatggccaggacttgggcacggccggcagctgcctgcgcaagttcagcacaatgcccttcctgttctgcaatattaacaacgtgtgcaactttgcatcacgaaatgactactcgtactggctgtccacccctgagcccatgcccatgtcaatggcacccatcacgggggaaaacataagaccatttattagtaggtgtgctgtgtgtgaggcgcctgccatggtgatggccgtgcacagccagaccattcagatcccaccgtgccccagcgggtggtcctcgctgtggatcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataatgaagcctgactcagctaatgtcacaacatggtgctacttcttcttctttttgttaacagcaacgaaccctagaaatatatcctgtgtacctcactgtccaatatgaaaaccgtaaagtgccttataggaatttgcgtaactaacacaccctgcttcattgacctctacttgctgaaggagaaaaagacagcgataagctttcaatagtggcataccaaatggcacttttgatgaaataaaatatcaatattttctgcaatccaatgcactgatgtgtgaagtgagaactccatcagaaaaccaaagggtgctaggaggtgtgggtgccttccatactgtttgcccattttcattcttgtattataattaattttctacccccagagataaatgtttgtttatatcactgtctagctgtttcaaaatttaggtcccttggtctgtacaaataatagcaatgtaaaaatggttttttgaacctccaaatggaattacagactcagtagccatatcttccaaccccccagtataaatttctgtctttctgctatgtgtggtactttgcagctgcttttgcagaaatcacaattttcctgtggaataaagatggtccaaaaatagtcaaaaattaaatatatatatatattagtaatttatatagatgtcagcaattaggcagatcaaggtttagtttaacttccactgttaaaataaagcttacatagttttcttcctttgaaagactgtgctgtcctttaacataggtttttaaagactaggatattgaatgtgaaacatccgttttcattgttcacttctaaaccaaaaattatgtgttgccaaaaccaaacccaggttcatgaatatggtgtctattatagtgaaacatgtactttgagcttattgtttttattctgtattaaatattttcagggttttaaacactaatcacaaactgaatgacttgacttcaaaagcaacaaccttaaaggccgtcatttcattagtattcctcattctgcatcctggcttgaaaaacagctctgttgaatcacagtatcagtattttcacacgtaagcacattcgggccatttccgtggtttctcatgagctgtgttcacagacctcagcagggcatcgcatggaccgcaggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgcataccttaaaaaaaaaaaaaaaaaa

SEQ ID NO: 14 - protein, 1669 aa, Homo Sapien
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMTGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 15 - DNA, 2582 bp, Homo Sapien
agcgttgtttttccttggcagctgcggagacccgtgataattcgttaactaattcaacaaacgggacccttctgtgtgccagaaaccgcaagcagttgctaacccagtgggacaggcggattggaagagcgggaaggtcctggcccagagcagtgtggtgagcgctgtgctggaagggaatgcgggcagtgggtacttggtagagcactgactgcctccggccagaggacttcccggaggaggtgacccatgagctggagtggtcagaggaaggctggcaaaagggcatcgtggacagaggaacagcctatgtgagtgggagcagagaccttggccaatgccattccttatggccttgtagtggaagcaaggtgatggggaaggaacactgtaggggatagctgtccacggacgctgtctacaagaccctggagtgagataacgtgcctggtactgtgccctgcatgtgtaagatgcccagttgaccttcgcagcaggagcctggatcagggcacttcctgcctcaggtattgctggacagcccagacacttccctctgtgaccatgaaactctgggtgtctgcattgctgatggcctggtttggtgtcctgagctgtgtgcaggccgaattcttcacctctattgggcacatgactgacctgatttatgcagagaaagagctggtgcagtctctgaaagagtacatccttgtggaggaagccaagctttccaagattaagagctgggccaacaaaatggaagccttgactagcaagtcagctgctgatgctgagggctacctggctcaccctgtgaatgcctacaaactggtgaagcggctaaacacagactggcctgcgctggaggaccttgtcctgcaggactcagctgcaggttttatcgccaacctctctgtgcagcggcagttcttccccactgatgaggacgagataggagctgccaaagccctgatgagacttcaggacacatacaggctggacccaggcacaatttccagaggggaacttccaggaaccaagtaccaggcaatgctgagtgtggatgactgctttgggatgggccgctcggcctacaatgaaggggactattatcatacggtgttgtggatggagcaggtgctaaagcagcttgatgccggggaggaggccaccacaaccaagtcacaggtgctggactacctcagctatgctgtcttccagttgggtgatctgcaccgtgccctggagctcacccgccgcctgctctcccttgacccaagccacgaacgagctggagggaatctgcggtactttgagcagttattggaggaagagagagaaaaaacgttaacaaatcagacagaagctgagctagcaaccccagaaggcatctatgagaggcctgtggactacctgcctgagagggatgtttacgagagcctctgtcgtggggagggtgtcaaactgacaccccgtagacagaagaggcttttctgtaggtaccaccatggcaacagggccccacagctgctcattgcccccttcaaagaggaggacgagtgggacagcccgcacatcgtcaggtactacgatgtcatgtctgatgaggaaatcgagaggatcaaggagatcgcaaaacctaaacttgcacgagccaccgttcgtgatcccaagacaggagtcctcactgtcgccagctaccgggtttccaaaagctcctggctagaggaagatgatgaccctgttgtggcccgagtaaatcgtcggatgcagcatatcacagggttaacagtaaagactgcagaattgttacaggttgcaaattatggagtgggaggacagtatgaaccgcacttcgacttctctaggcgaccttttgacagcggcctcaaaacagaggggaataggttagcgacgtttcttaactacatgagtgatgtagaagctggtggtgccaccgtcttccctgatctgggggctgcaatttggcctaagaagggtacagctgtgttctggtacaacctcttgcggagcggggaaggtgactaccgaacaagacatgctgcctgccctgtgcttgtgggctgcaagtgggtctccaataagtggttccatgaacgaggacaggagttcttgagaccttgtggatcaacagaagttgactgacatccttttctgtccttccccttcctggtccttcagcccatgtcaacgtgacagacacctttgtatgttcctttgtatgttcctatcaggctgatttttggagaaatgaatgtttgtctggagcagagggagaccatactagggcgactcctgtgtgactgaagtcccagcccttccattcagcctgtgccatccctggccccaaggctaggatcaaagtggctgcagcagagttagctgtctagcgcctagcaaggtgcctttgtacctcaggtgttttaggtgtgagatgtttcagtgaaccaaagttctgataccttgtttacatgtttgtttttatggcatttctatctattgtggctttaccaaaaaataaaatgtccctaccagaagccttaaaaaaaaaaaaaaaaaaa

SEQ ID NO: 16 - protein, 535 aa, Homo Sapien
MKLWVSALLMAWFGVLSCVQAEFFTSIGHMTDLIYAEKELVQSLKEYILVEEAKLSKIKSWANKMEALTSKSAADAEGYLAHPVNAYKLVKRLNTDWPALEDLVLQDSAAGFIANLSVQRQFFPTDEDEIGAAKALMRLQDTYRLDPGTISRGELPGTKYQAMLSVDDCFGMGRSAYNEGDYYHTVLWMEQVLKQLDAGEEATTTKSQVLDYLSYAVFQLGDLHRALELTRRLLSLDPSHERAGGNLRYFEQLLEEEREKTLTNQTEAELATPEGIYERPVDYLPERDVYESLCRGEGVKLTPRRQKRLFCRYHHGNRAPQLLIAPFKEEDEWDSPHIVRYYDVMSDEEIERIKEIAKPKLARATVRDPKTGVLTVASYRVSKSSWLEEDDDPVVARVNRRMQHITGLTVKTAELLQVANYGVGGQYEPHFDFSRNDERDTFKHLGTGNRVATFLNYMSDVEAGGATVFPDLGAAIWPKKGTAVFWYNLLRSGEGDYRTRHAACPVLVGCKWVSNKWFHERGQEFLRPCGSTEVD

SEQ ID NO: 17 - protein, 533 aa, Homo Sapien
MKLWVSALLMAWFGVLSCVQAEFFTSIGHMTDLIYAEKELVQSLKEYILVEEAKLSKIKSWANKMEALTSKSAADAEGYLAHPVNAYKLVKRLNTDWPALEDLVLQDSAAGFIANLSVQRQFFPTDEDEIGAAKALMRLQDTYRLDPGTISRGELPGTKYQAMLSVDDCFGMGRSAYNEGDYYHTVLWMEQVLKQLDAGEEATTTKSQVLDYLSYAVFQLGDLHRALELTRRLLSLDPSHERAGGNLRYFEQLLEEEREKTLTNQTEAELATPEGIYERPVDYLPERDVYESLCRGEGVKLTPRRQKRLFCRYHHGNRAPQLLIAPFKEEDEWDSPHIVRYYDVMSDEEIERIKEIAKPKLARATVRDPKTGVLTVASYRVSKSSWLEEDDDPVVARVNRRMQHITGLTVKTAELLQVANYGVGGQYEPHFDFSRRPFDSGLKTEGNRLATFLNYMSDVEAGGATVFPDLGAAIWPKKGTAVFWYNLLRSGEGDYRTRHAACPVLVGCKWVSNKWFHERGQEFLRPCGSTEVD

SEQ ID NO: 18 - DNA, 5826 bp, Homo Sapien
gaggaggagacggcatccagtacagaggggctggacttggacccctgcagcagccctgcacaggagaagcggcatataaagccgcgctgcccgggagccgctcggccacgtccaccggagcatcctgcactgcagggccggtctctcgctccagcagagcctgcgcctttctgactcggtccggaacactgaaaccagtcatcactgcatctttttggcaaaccaggagctcagctgcaggaggcaggatggtctggaggctggtcctgctggctctgtgggtgtggcccagcacgcaagctggtcaccaggacaaagacacgaccttcgaccttttcagtatcagcaacatcaaccgcaagaccattggcgccaagcagttccgcgggcccgaccccggcgtgccggcttaccgcttcgtgcgctttgactacatcccaccggtgaacgcagatgacctcagcaagatcaccaagatcatgcggcagaaggagggcttcttcctcacggcccagctcaagcaggacggcaagtccaggggcacgctgttggctctggagggccccggtctctcccagaggcagttcgagatcgtctccaacggccccgcggacacgctggatctcacctactggattgacggcacccggcatgtggtctccctggaggacgtcggcctggctgactcgcagtggaagaacgtcaccgtgcaggtggctggcgagacctacagcttgcacgtgggctgcgacctcatagacagcttcgctctggacgagcccttctacgagcacctgcaggcggaaaagagccggatgtacgtggccaaaggctctgccagagagagtcacttcaggggtttgcttcagaacgtccacctagtgtttgaaaactctgtggaagatattctaagcaagaagggttgccagcaaggccagggagctgagatcaacgccatcagtgagaacacagagacgctgcgcctgggtccgcatgtcaccaccgagtacgtgggccccagctcggagaggaggcccgaggtgtgcgaacgctcgtgcgaggagctgggaaacatggtccaggagctctcggggctccacgtcctcgtgaaccagctcagcgagaacctcaagagagtgtcgaatgataaccagtttctctgggagctcattggtggccctcctaagacaaggaacatgtcagcttgctggcaggatggccggttctttgcggaaaatgaaacgtgggtggtggacagctgcaccacgtgtacctgcaagaaatttaaaaccatttgccaccaaatcacctgcccgcctgcaacctgcgccagtccatcctttgtggaaggcgaatgctgcccttcctgcctccactcggtggacggtgaggagggctggtctccgtgggcagagtggacccagtgctccgtgacgtgtggctctgggacccagcagagaggccggtcctgtgacgtcaccagcaacacctgcttggggccctccatccagacacgggcttgcagtctgagcaagtgtgacacccgcatccggcaggacggcggctggagccactggtcaccttggtcttcatgctctgtgacctgtggagttggcaatatcacacgcatccgtctctgcaactccccagtgccccagatggggggcaagaattgcaaagggagtggccgggagaccaaagcctgccagggcgccccatgcccaatcgatggccgctggagcccctggtccccgtggtcggcctgcactgtcacctgtgccggtgggatccgggagcgcacccgggtctgcaacagccctgagcctcagtacggagggaaggcctgcgtgggggatgtgcaggagcgtcagatgtgcaacaagaggagctgccccgtggatggctgtttatccaacccctgcttcccgggagcccagtgcagcagcttccccgatgggtcctggtcatgcggctcctgccctgtgggcttcttgggcaatggcacccactgtgaggacctggacgagtgtgccctggtccccgacatctgcttctccaccagcaaggtgcctcgctgtgtcaacactcagcctggcttccactgcctgccctgcccgccccgatacagagggaaccagcccgtcggggtcggcctggaagcagccaagacggaaaagcaagtgtgtgagcccgaaaacccatgcaaggacaagacacacaactgccacaagcacgcggagtgcatctacctgggccacttcagcgaccccatgtacaagtgcgagtgccagacaggctacgcgggcgacgggctcatctgcggggaggactcggacctggacggctggcccaacctcaatctggtctgcgccaccaacgccacctaccactgcatcaaggataactgcccccatctgccaaattctgggcaggaagactttgacaaggacgggattggcgatgcctgtgatgatgacgatgacaatgacggtgtgaccgatgagaaggacaactgccagctcctcttcaatccccgccaggctgactatgacaaggatgaggttggggaccgctgtgacaactgcccttacgtgcacaaccctgcccagatcgacacagacaacaatggagagggtgacgcctgctccgtggacattgatggggacgatgtcttcaatgaacgagacaattgtccctacgtctacaacactgaccagagggacacggatggtgacggtgtgggggatcactgtgacaactgccccctggtgcacaaccctgaccagaccgacgtggacaatgaccttgttggggaccagtgtgacaacaacgaggacatagatgacgacggccaccagaacaaccaggacaactgcccctacatctccaacgccaaccaggctgaccatgacagagacggccagggcgacgcctgtgaccctgatgatgacaacgatggcgtccccgatgacagggacaactgccggcttgtgttcaacccagaccaggaggacttggacggtgatggacggggtgatatttgtaaagatgattttgacaatgacaacatcccagatattgatgatgtgtgtcctgaaaacaatgccatcagtgagacagacttcaggaacttccagatggtccccttggatcccaaagggaccacccaaattgatcccaactgggtcattcgccatcaaggcaaggagctggttcagacagccaactcggaccccggcatcgctgtaggttttgacgagtttgggtctgtggacttcagtggcacattctacgtaaacactgaccgggacgacgactatgccggcttcgtctttggttaccagtcaagcagccgcttctatgtggtgatgtggaagcaggtgacgcagacctactgggaggaccagcccacgcgggcctatggctactccggcgtgtccctcaaggtggtgaactccaccacggggacgggcgagcacctgaggaacgcgctgtggcacacggggaacacgccggggcaggtgcgaaccttatggcacgaccccaggaacattggctggaaggactacacggcctataggtggcacctgactcacaggcccaagactggctacatcagagtcttagtgcatgaaggaaaacaggtcatggcagactcaggacctatctatgaccaaacctacgctggcgggcggctgggtctatttgtcttctctcaagaaatggtctatttctcagacctcaagtacgaatgcagagatatttaaacaagatttgctgcatttccggcaatgccctgtgcatgccatggtccctagacacctcagttcattgtggtccttgtggcttctctctctagcagcacctcctgtcccttgaccttaactctgatggttcttcacctcctgccagcaaccccaaacccaagtgccttcagaggataaatatcaatggaactcagagatgaacatctaacccactagaggaaaccagtttggtgatatatgagactttatgtggagtgaaaattgggcatgccattacattgctttttcttgtttgtttaaaaagaatgacgtttacatataaaatgtaattacttattgtatttatgtgtatatggagttgaagggaatactgtgcataagccattatgataaattaagcatgaaaaatattgctgaactacttttggtgcttaaagttgtcactattcttgaattagagttgctctacaatgacacacaaatcccattaaataaattataaacaagggtcaattcaaatttgaagtaatgttttagtaaggagagattagaagacaacaggcatagcaaatgacataagctaccgattaactaatcggaacatgtaaaacagttacaaaaataaacgaactctcctcttgtcctacaatgaaagccctcatgtgcagtagagatgcagtttcatcaaagaacaaacatccttgcaaatgggtgtgacgcggttccagatgtggatttggcaaaacctcatttaagtaaaaggttagcagagcaaagtgcggtgctttagctgctgcttgtgccgctgtggcgtcggggaggctcctgcctgagcttccttccccagctttgctgcctgagaggaaccagagcagacgcacaggccggaaaaggcgcatctaacgcgtatctaggctttggtaactgcggacaagttgcttttacctgatttgatgatacatttcattaaggttccagttataaatattttgttaatatttattaagtgactatagaatgcaactccatttaccagtaacttattttaaatatgcctagtaacacatatgtagtataatttctagaaacaaacatctaataagtatataatcctgtgaaaatatgaggcttgataatattaggttgtcacgatgaagcatgctagaagctgtaacagaatacatagagaataatgaggagtttatgatggaaccttaaatatataatgttgccagcgattttagttcaatatttgttactgttatctatctgctgtatatggaattcttttaattcaaacgctgaaaagaatcagcatttagtcttgccaggcacacccaataatcagtcatgtgtaatatgcacaagtttgtttttgtttttgttttttttgttggttggtttgtttttttgctttaagttgcatgatctttctgcaggaaatagtcactcatcccactccacataaggggtttagtaagagaagtctgtctgtctgatgatggatagggggcaaatctttttcccctttctgttaatagtcatcacatttctatgccaaacaggaacaatccataactttagtcttaatgtacacattgcattttgataaaattaattttgttgtttcctttgaggttgatcgttgtgttgttgttttgctgcactttttacttttttgcgtgtggagctgtattcccgagaccaacgaagcgttgggatacttcattaaatgtagcgactgtcaacagcgtgcaggttttctgtttctgtgttgtggggtcaaccgtacaatggtgtgggagtgacgatgatgtgaatatttagaatgtaccatattttttgtaaattatttatgtttttctaaacaaatttatcgtataggttgatgaaacgtcatgtgttttgccaaagactgtaaatatttatttatgtgttcacatggtcaaaatttcaccactgaaaccctgcacttagctagaacctcatttttaaagattaacaacaggaaataaattgtaaaaaaggttttctatacatgaaaaaaaaaaaaaaaaaa

SEQ ID NO: 19 - protein, 1172 aa, Homo Sapien
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 20 - DNA, 4531 bp, Homo Sapien
tggtcgtcctccttgggttcgggtgaaagcgcttgggggttcagtgggccatgatccccgagctgctggagaactgaaggcggacagtctcctgcgaaaccagcggagctggagtttgttcagatcatcatcatcgtggtggtgatgatggtgatggtggtggtgatcacgtgcctgctgagccactacaagctgtctgcacggtccttcatcagccggcacagccaggggcggaggagagaagatgccctgtcctcagaaggatgcctgtggccctcggagagcacagtgtcaggcaacggaatcccagagccgcaggtctacgccccgcctcggcccaccgaccgcctggccgtgccgcccttcgcccagcgggagcgcttccaccgcttccagcccacctatccgtacctgcagcacgagatcgacctgccacccaccatctcgctgtcagacggggaggagcccccaccctaccagggcccctgcaccctccagcttcgggaccccgagcagcagctggaactgaaccgggagtcggtgcgcgcacccccaaacagaaccatcttcgacagtgacctgatggatagtgccaggctgggcggcccctgcccccccagcagtaactcgggcatcagcgccacgtgctacggcagcggcgggcgcatggaggggccgccgcccacctacagcgaggtcatcggccactacccggggtcctccttccagcaccagcagagcagtgggccgccctccttgctggaggggacccggctccaccacacacacatcgcgcccctagagagcgcagccatctggagcaaagagaaggataaacagaaaggacaccctctctagggtccccaggggggccgggctggggctgcgtaggtgaaaaggcagaacactccgcgcttcttagaagaggagtgagaggaaggcggggggcgcagcaacgcatcgtgtggccctcccctcccacctccctgtgtataaatatttacatgtgatgtctggtctgaatgcacaagctaagagagcttgcaaaaaaaaaaagaaaaaagaaaaaaaaaaaccacgtttctttgttgagctgtgtcttgaaggcaaaagaaaaaaaatttctacagtagtctttcttgtttctagttgagctgcgtgcgtgaatgcttattttcttttgtttatgataatttcacttaactttaaagacatatttgcacaaaacctttgtttaaagatctgcaatattatatatataaatatatataagataagagaaactgtatgtgcgagggcaggagtatttttgtattagaagaggcctattaaaaaaaaaagttgttttctgaactagaagaggaaaaaaatggcaatttttgagtgccaagtcagaaagtgtgtattaccttgtaaagaaaaaaattacaaagcaggggtttagagttatttatataaatgttgagattttgcactattttttaatataaatatgtcagtgcttgcttgatggaaacttctcttgtgtctgttgagactttaagggagaaatgtcggaatttcagagtcgcctgacggcagagggtgagcccccgtggagtctgcagagaggccttggccaggagcggcgggctttcccgaggggccactgtccctgcagagtggatgcttctgcctagtgacaggttatcaccacgttatatattccctaccgaaggagacaccttttcccccctgacccagaacagcctttaaatcacaagcaaaataggaaagttaaccacggaggcaccgagttccaggtagtggttttgcctttcccaaaaatgaaaataaactgttaccgaaggaattagtttttcctcttcttttttccaactgtgaaggtccccgtggggtggagcatggtgcccctcacaagccgcagcggctggtgcccgggctaccagggacatgccagagggctcgatgacttgtctctgcagggcgctttggtggttgttcagctggctaaaggttcaccggtgaaggcaggtgcggtaactgccgcactggaccctaggaagccccaggtattcgcaatctgacctcctcctgtctgtttcccttcacggatcaattctcacttaagaggccaataaacaacccaacatgaaaaggtgacaagcctgggtttctcccaggataggtgaaagggttaaaatgagtaaagcagttgagcaaacaccaacccgagcttcgggcgcagaattcttcaccttctcttcccctttccatctcctttccccgcggaaacaacgcttcccttctggtgtgtctgttgatctgtgttttcatttacatctctcttagactccgctcttgttctccaggttttcaccagatagatttggggttggcgggacctgctggtgacgtgcaggtgaaggacaggaaggggcatgtgagcgtaaatagaggtgaccagaggagagcatgaggggtggggctttgggacccaccggggccagtggctggagcttgacgtctttcctccccatgggggtgggagggcccccagctggaagagcagactcccagctgctaccccctcccttcccatgggagtggctttccattttgggcagaatgctgactagtagactaacataaaagatataaaaggcaataactattgtttgtgagcaacttttttataacttccaaaacaaaaacctgagcacagttttgaagttctagccactcgagctcatgcatgtgaaacgtgtgctttacgaaggtggcagctgacagacgtgggctctgcatgccgccagcctagtagaaagttctcgttcattggcaacagcagaacctgcctctccgtgaagtcgtcagcctaaaatttgtttctctcttgaagaggattctttgaaaaggtcctgcagagaaatcagtacaggttatcccgaaaggtacaaggacgcacttgtaaagatgattaaaacgtatctttcctttatgtgacgcgtctctagtgccttactgaagaagcagtgacactcccgtcgctcggtgaggacgttcccggacagtgcctcactcacctgggactggtatcccctcccagggtccaccaagggctcctgcttttcagacaccccatcatcctcgcgcgtcctcaccctgtctctaccagggaggtgcctagcttggtgaggttactcctgctcctccaacctttttttgccaaggtttgtacacgactcccatctaggctgaaaacctagaagtggaccttgtgtgtgtgcatggtgtcagcccaaagccaggctgagacagtcctcatatcctcttgagccaaactgtttgggtctcgttgcttcatggtatggtctggatttgtgggaatggctttgcgtgagaaaggggaggagagtggttgctgccctcagccggcttgaggacagagcctgtccctctcatgacaactcagtgttgaagcccagtgtcctcagcttcatgtccagtggatggcagaagttcatggggtagtggcctctcaaaggctgggcgcatcccaagacagccagcaggttgtctctggaaacgaccagagttaagctctcggcttctctgctgagggtgcaccctttcctctagatggtagttgtcacgttatctttgaaaactcttggactgctcctgaggaggccctcttttccagtaggaagttagatgggggttctcagaagtggctgattggaaggggacaagcttcgtttcaggggtctgccgttccatcctggttcagagaaggccgagcgtggctttctctagccttgtcactgtctccctgcctgtcaatcaccacctttcctccagaggaggaaaattatctcccctgcaaagcccggttctacacagatttcacaaattgtgctaagaaccgtccgtgttctcagaaagcccagtgtttttgcaaagaatgaaaagggaccccatatgtagcaaaaatcagggctgggggagagccgggttcattccctgtcctcattggtcgtccctatgaattgtacgtttcagagaaattttttttcctatgtgcaacacgaagcttccagaaccataaaatatcccgtcgataaggaaagaaaatgtcgttgttgttgtttttctggaaactgcttgaaatcttgctgtactatagagctcagaaggacacagcccgtcctcccctgcctgcctgattccatggctgttgtgctgattccaatgctttcacgttggttcctggcgtgggaactgctctcctttgcagccccatttcccaagctctgttcaagttaaacttatgtaagctttccgtggcatgcggggcgcgcacccacgtccccgctgcgtaagactctgtatttggatgccaatccacaggcctgaagaaactgcttgttgtgtatcagtaatcattagtggcaatgatgacattctgaaaagctgcaatacttatacaataaattttacaattctttggaaaaaaaaaaaaaaaaaa

SEQ ID NO: 21 - protein, 237 aa, Homo Sapien
MMVMVVVITCLLSHYKLSARSFISRHSQGRRREDALSSEGCLWPSESTVSGNGIPEPQVYAPPRPTDRLAVPPFAQRERFHRFQPTYPYLQHEIDLPPTISLSDGEEPPPYQGPCTLQLRDPEQQLELNRESVRAPPNRTIFDSDLMDSARLGGPCPPSSNSGISATCYGSGGRMEGPPPTYSEVIGHYPGSSFQHQQSSGPPSLLEGTRLHHTHIAPLESAAIWSKEKDKQKGHPL

SEQ ID NO: 22 - DNA, 6821 bp, Homo Sapien
gggcgcagtcgggagccggccgtggtggctccgtgcgtccgagcgtccgtccgcgccgtcggccatggccaagcgctccaggggccccgggcgccgctgcctgttggcgctcgtgctgttctgcgcctgggggacgctggccgtggtggcccagaagccgggcgcagggtgtccgagccgctgcctgtgcttccgcaccaccgtgcgctgcatgcatctgctgctggaggccgtgcccgccgtggcgccgcagacctccatcctagatcttcgctttaacagaatcagagagatccaacctggggcattcaggcggctgaggaacttgaacacattgcttctcaataataatcagatcaagaggatacctagtggagcatttgaagacttggaaaatttaaaatatctctatctgtacaagaatgagatccagtcaattgacaggcaagcatttaagggacttgcctctctagagcaactatacctgcactttaatcagatagaaactttggacccagattcgttccagcatctcccgaagctcgagaggctatttttgcataacaaccggattacacatttagttccagggacatttaatcacttggaatctatgaagagattgcgactggactcaaacacacttcactgcgactgtgaaatcctgtggttggcggatttgctgaaaacctacgcggagtcggggaacgcgcaggcagcggccatctgtgaatatcccagacgcatccagggacgctcagtggcaaccatcaccccggaagagctgaactgtgaaaggccccggatcacctccgagccccaggacgcagatgtgacctcggggaacaccgtgtacttcacctgcagagccgaaggcaaccccaagcctgagatcatctggctgcgaaacaataatgagctgagcatgaagacagattcccgcctaaacttgctggacgatgggaccctgatgatccagaacacacaggagacagaccagggtatctaccagtgcatggcaaagaacgtggccggagaggtgaagacgcaagaggtgaccctcaggtacttcgggtctccagctcgacccacttttgtaatccagccacagaatacagaggtgctggttggggagagcgtcacgctggagtgcagcgccacaggccaccccccgccgcggatctcctggacgagaggtgaccgcacacccttgccagttgacccgcgggtgaacatcacgccttctggcgggctttacatacagaacgtcgtacagggggacagcggagagtatgcgtgctctgcgaccaacaacattgacagcgtccatgccaccgctttcatcatcgtccaggctcttcctcagttcactgtgacgcctcaggacagagtcgttattgagggccagaccgtggatttccagtgtgaagccaagggcaacccgccgcccgtcatcgcctggaccaagggagggagccagctctccgtggaccggcggcacctggtcctgtcatcgggaacacttagaatctctggtgttgccctccacgaccagggccagtacgaatgccaggctgtcaacatcatcggctcccagaaggtcgtggcccacctgactgtgcagcccagagtcaccccagtgtttgccagcattcccagcgacacaacagtggaggtgggcgccaatgtgcagctcccgtgcagctcccagggcgagcccgagccagccatcacctggaacaaggatggggttcaggtgacagaaagtggaaaatttcacatcagccctgaaggattcttgaccatcaatgacgttggccctgcagacgcaggtcgctatgagtgtgtggcccggaacaccattgggtcggcctcggtgagcatggtgctcagtgtgaatgttcctgacgtcagtcgaaatggagatccgtttgtagctacctccatcgtggaagcgattgcgactgttgacagagctataaactcaacccgaacacatttgtttgacagccgtcctcgttctccaaatgatttgctggccttgttccggtatccgagggatccttacacagttgaacaggcacgggcgggagaaatctttgaacggacattgcagctcattcaggagcatgtacagcatggcttgatggtcgacctcaacggaacaagttaccactacaacgacctggtgtctccacagtacctgaacctcatcgcaaacctgtcgggctgtaccgcccaccggcgcgtgaacaactgctcggacatgtgcttccaccagaagtaccggacgcacgacggcacctgtaacaacctgcagcaccccatgtggggcgcctcgctgaccgccttcgagcgcctgctgaaatccgtgtacgagaatggcttcaacacccctcggggcatcaacccccaccgactgtacaacgggcacgcccttcccatgccgcgcctggtgtccaccaccctgatcgggacggagaccgtcacacccgacgagcagttcacccacatgctgatgcagtggggccagttcctggaccacgacctcgactccacggtggtggccctgagccaggcacgcttctccgacggacagcactgcagcaacgtgtgcagcaacgaccccccctgcttctctgtcatgatcccccccaatgactcccgggccaggagcggggcccgctgcatgttcttcgtgcgctccagccctgtgtgcggcagcggcatgacttcgctgctcatgaactccgtgtacccgcgggagcagatcaaccagctcacctcctacatagacgcatccaacgtgtacgggagcacggagcatgaggcccgcagcatccgcgacctggccagccaccgcggcctgctgcggcagggcatcgtgcagcggtccgggaagccgctgctccccttcgccaccgggccgcccacggagtgcatgcgggacgagaacgagagccccatcccctgcttcctggccggggaccaccgcgccaacgagcagctgggcctgaccagcatgcacacgctgtggttccgcgagcacaaccgcattgccacggagctgctcaagctgaacccgcactgggacggcgacaccatctactatgagaccaggaagatcgtgggtgcggagatccagcacatcacctaccagcactggctcccgaagatcctgggggaggtgggcatgaggacgctgggagagtaccacggctacgaccccggcatcaatgctggcatcttcaacgccttcgccaccgcggccttcaggtttggccacacgcttgtcaacccactgctttaccggctggacgagaacttccagcccattgcacaagatcacctcccccttcacaaagctttcttctctcccttccggattgtgaatgagggcggcatcgatccgcttctcagggggctgttcggggtggcggggaaaatgcgtgtgccctcgcagctgctgaacacggagctcacggagcggctgttctccatggcacacacggtggctctggacctggcggccatcaacatccagcggggccgggaccacgggatcccaccctaccacgactacagggtctactgcaatctatcggcggcacacacgttcgaggacctgaaaaatgagattaaaaaccctgagatccgggagaaactgaaaaggttgtatggctcgacactcaacatcgacctgtttccggcgctcgtggtggaggacctggtgcctggcagccggctgggccccaccctgatgtgtcttctcagcacacagttcaagcgcctgcgagatggggacaggttgtggtatgagaaccctggggtgttctccccggcccagctgactcagatcaagcagacgtcgctggccaggatcctatgcgacaacgcggacaacatcacccgggtgcagagcgacgtgttcagggtggcggagttccctcacggctacggcagctgtgacgagatccccagggtagacctccgggtgtggcaggactgctgtgaagactgtaggaccagggggcagttcaatgccttttcctatcatttccgaggcagacggtctcttgagttcagctaccaggaggacaagccgaccaagaaaacaagaccacggaaaatacccagtgttgggagacagggggaacatctcagcaacagcacctcagccttcagcacacgctcagatgcatctgggacaaatgacttcagagagtttgttctggaaatgcagaagaccatcacagacctcagaacacagataaagaaacttgaatcacggctcagtaccacagagtgcgtggatgccgggggcgaatctcacgccaacaacaccaagtggaaaaaagatgcatgcaccatttgtgaatgcaaagacgggcaggtcacctgcttcgtggaagcttgcccccctgccacctgtgctgtccccgtgaacatcccaggggcctgctgtccagtctgcttacagaagagggcggaggaaaagccctaggctcctgggaggctcctcagagtttgtctgctgtgccatcgtgagatcgggtggccgatggcagggagctgcggactgcagaccaggaaacacccagaactcgtgacatttcatgacaacgtccagctggtgctgttacagaaggcagtgcaggaggcttccaaccagagcatctgcggagaaggaggcacagcaggtgcctgaagggaagcaggcaggagtcctagcttcacgttagacttctcaggtttttatttaattcttttaaaatgaaaaattggtgctactattaaattgcacagttgaatcatttaggcgcctaaattgattttgcctcccaacaccatttctttttaaataaagcaggatacctctatatgtcagccttgccttgttcagatgccaggagccggcagacctgtcacccgcaggtggggtgagtcttggagctgccagaggggctcaccgaaatcggggttccatcacaagctatgtttaaaaagaaaattggtgtttggcaaacggaacagaacctttgatgagagcgttcacagggacactgtctgggggtgcagtgcaagcccccggcctcttccctgggaacctctgaactcctccttcctctgggctctctgtaacatttcaccacacgtcagcatctaatcccaagacaaacattcccgctgctcgaagcagctgtatagcctgtgactctccgtgtgtcagctccttccacacctgattagaacattcataagccacatttagaaacaggtttgctttcagctgtcacttgcacacatactgcctagttgtgaaccaaatgtgaaaaaacctccttcatcccattgtgtatctgatacctgccgagggccaagggtgtgtgttgacaacgccgctcccagccggccctggttgcgtccacgtcctgaacaagagccgcttccggatggctcttcccaagggaggaggagctcaagtgtcgggaactgtctaacttcaggttgtgtgagtgcgttaaaaaaaaaaaaaaaaaagaatccctatacctcatttgtatttttaaaatgcgtgatgttttatgaaattgtgtccattttttaggtattagatatggcagaaaaaccatttccactatgcaaagttcttttagacgtcagtgaaaatcaactctcatacctcatggtctctctttaattgaccaaaaccttccatttttctctaaatacaaagcgatctgtgttctgagcaacctttccccgaacacacagcttcagtgcagcacgctgacctgagtatccaccatgtgccaggcacagtgctgggcacacgaggcaccaaggtccgggccacctgcccgcagcaaggcccagctgaggtggtggagggagcccctgaggtcaggggccgtttcggttcagggtggcaggtgtccagcactggggtatggcgtcgaggcttccatggggtgggggaggccagcttccttctgacaggatgggcgcatacagtgcctggtgtgatttgtgcacaacccgtgttccaggtgcacatcctcccaaggagacacccagacccttccagcacgggccggccaagttgctgcggcggaggcagcatttcagctgtgaggaaggtcattggattcatgtgttttatctgtaaaaatggttgtcttaacttcttaacctcatattggtaagtgattgataaaaattggttggtgtttcatgacatgtggacttcttttgaaatagcaagtcaaatgtagtgaccaaattgtggaagagatttctgtcaaataggaaatgtgtaagttcgtctaaaagctgatggttatgtaagttgctcaggcactcagatgacagcagattctgggttctgggagtgttctgtgcctcttacatgccctggaggcctcatggtctcagtgctgaggcggcacacctgtagcacacctgcgtaatgtgcggtctgggccagtcacaaggaattgtgttgtctaagccaaagggggaagctgactgtgatttaccaaaaaaaattctgtaattcaaaccaaaatgtctgcggaatcaccagtttgatactctctgtaatcagaacagtgggcagtgcctgggtgaacgtgtctagcagccactgtgcgggatcgctgtaacaggagtggaatgtacatatttatttacttttctaactgctccaacagccaaatgccttttttatgaccattgtattcagttcattaccaaagaaatgtttgcactttgtaatgatgcctttcagttcaaataaatgggtcacattttcaaatgga

SEQ ID NO: 23 - protein, 1479 aa, Homo Sapien
MAKRSRGPGRRCLLALVLFCAWGTLAVVAQKPGAGCPSRCLCFRTTVRCMHLLLEAVPAVAPQTSILDLRFNRIREIQPGAFRRLRNLNTLLLNNNQIKRIPSGAFEDLENLKYLYLYKNEIQSIDRQAFKGLASLEQLYLHFNQIETLDPDSFQHLPKLERLFLHNNRITHLVPGTFNHLESMKRLRLDSNTLHCDCEILWLADLLKTYAESGNAQAAAICEYPRRIQGRSVATITPEELNCERPRITSEPQDADVTSGNTVYFTCRAEGNPKPEIIWLRNNNELSMKTDSRLNLLDDGTLMIQNTQETDQGIYQCMAKNVAGEVKTQEVTLRYFGSPARPTFVIQPQNTEVLVGESVTLECSATGHPPPRISWTRGDRTPLPVDPRVNITPSGGLYIQNVVQGDSGEYACSATNNIDSVHATAFIIVQALPQFTVTPQDRVVIEGQTVDFQCEAKGNPPPVIAWTKGGSQLSVDRRHLVLSSGTLRISGVALHDQGQYECQAVNIIGSQKVVAHLTVQPRVTPVFASIPSDTTVEVGANVQLPCSSQGEPEPAITWNKDGVQVTESGKFHISPEGFLTINDVGPADAGRYECVARNTIGSASVSMVLSVNVPDVSRNGDPFVATSIVEAIATVDRAINSTRTHLFDSRPRSPNDLLALFRYPRDPYTVEQARAGEIFERTLQLIQEHVQHGLMVDLNGTSYHYNDLVSPQYLNLIANLSGCTAHRRVNNCSDMCFHQKYRTHDGTCNNLQHPMWGASLTAFERLLKSVYENGFNTPRGINPHRLYNGHALPMPRLVSTTLIGTETVTPDEQFTHMLMQWGQFLDHDLDSTVVALSQARFSDGQHCSNVCSNDPPCFSVMIPPNDSRARSGARCMFFVRSSPVCGSGMTSLLMNSVYPREQINQLTSYIDASNVYGSTEHEARSIRDLASHRGLLRQGIVQRSGKPLLPFATGPPTECMRDENESPIPCFLAGDHRANEQLGLTSMHTLWFREHNRIATELLKLNPHWDGDTIYYETRKIVGAEIQHITYQHWLPKILGEVGMRTLGEYHGYDPGINAGIFNAFATAAFRFGHTLVNPLLYRLDENFQPIAQDHLPLHKAFFSPFRIVNEGGIDPLLRGLFGVAGKMRVPSQLLNTELTERLFSMAHTVALDLAAINIQRGRDHGIPPYHDYRVYCNLSAAHTFEDLKNEIKNPEIREKLKRLYGSTLNIDLFPALVVEDLVPGSRLGPTLMCLLSTQFKRLRDGDRLWYENPGVFSPAQLTQIKQTSLARILCDNADNITRVQSDVFRVAEFPHGYGSCDEIPRVDLRVWQDCCEDCRTRGQFNAFSYHFRGRRSLEFSYQEDKPTKKTRPRKIPSVGRQGEHLSNSTSAFSTRSDASGTNDFREFVLEMQKTITDLRTQIKKLESRLSTTECVDAGGESHANNTKWKKDACTICECKDGQVTCFVEACPPATCAVPVNIPGACCPVCLQKRAEEKP

SEQ ID NO: 24 - DNA, 100 bp, Homo Sapien
aaatgggcttgaagctgcttacgaatttgccgacagagatgaagtccggtttttcaaagggaataagtactgggctgttcagggacagaatgtgctacac

SEQ ID NO: 25 - DNA, 100 bp, Homo Sapien
tgggcttaagttttcaaggaccaaaaggtgacaagggtgaccaaggggtcagtgggcctccaggagtaccaggacaagctcaagttcaagaaaaaggaga

SEQ ID NO: 26 - DNA, 100 bp, Homo Sapien
tgtgcttgtgggctgcaagtgggtctccaataagtggttccatgaacgaggacaggagttcttgagaccttgtggatcaacagaagttgactgacatcct

SEQ ID NO: 27 - DNA, 100 bp, Homo Sapien
aaacatccttgcaaatgggtgtgacgcggttccagatgtggatttggcaaaacctcatttaagtaaaaggttagcagagcaaagtgcggtgctttagctg

SEQ ID NO: 28 - DNA, 100 bp, Homo Sapien
ttggcacaacaggaagctgttgaaggaggatgttcccatcttggtcagtcctatgcggatagagatgtctggaagccagaaccatgccaaatatgtgtct

SEQ ID NO: 29 - DNA, 100 bp, Homo Sapien; identical to SEQ ID NO: 25

SEQ ID NO: 30 - DNA, 100 bp, Homo Sapien
gtaaaggtcatcccaccatcaccaaagcctccgtttttaacaacctccaacacgatccatttagaggccaaatgtcattctgcaggtgccttcccgatgg

SEQ ID NO: 31 - DNA, 100 bp, Homo Sapien
ggttcatgctaccctgaagtcactcagtagtcagattgaaaccatgcgcagccccgatggctcgaaaaagcacccagcccgcacgtgtgatgacctaaag

SEQ ID NO: 32 - DNA, 100 bp, Homo Sapien
ctgtggaaggactttgtgaaggaattggtgctggattagtggatgttgctatctgggttggcacttgttcagattacccaaaaggagatgcttctactgg

SEQ ID NO: 33 - DNA, 100 bp, Homo Sapien
aggccctgcccttataggaacagaagaggaaagagagacacagctgcagaggccacctggattgtgcctaatgtgtttgagcatcgcttaggagaagtct

SEQ ID NO: 34 - DNA, 100 bp, Homo Sapien
gagaagatgtttgaaaaaactgactctgctaatgagcctggactcagagctcaagtctgaactctacctccagacagaatgaagttcatctcgacatctc

SEQ ID NO: 35 - DNA, 100 bp, Homo Sapien; identical to SEQ ID NO: 24

SEQ ID NO: 36 - DNA, 100 bp, Homo Sapien; identical to SEQ ID NO: 26

SEQ ID NO: 37 - DNA, 100 bp, Homo Sapien
ctccaggaaccagcgaagaccgctataagtctggcttgacaactctggtggcaacaagtgtcaacagtgtaacaggcattcgcatcgaggatctgccaac

SEQ ID NO: 38 - DNA, 100 bp, Homo Sapien
aaacattgcacttaataacgtgggagaagactttcagggaggtggttgcaaatttctaaggtacaattgctctattgagtcaccacgaaaaggctggagc

SEQ ID NO: 39 - DNA, 100 bp, Homo Sapien
agagacggtcacttcacactctttgctcccaccaatgaggcttttgagaaacttccacgaggtgtcctagaaaggatcatgggagacaaagtggcttccg

SEQ ID NO: 40 - DNA, 100 bp, Homo Sapien
tggaggggcaggcttgcgagctgcatttggcctttctgaggcagggtttaatacagcatgtgttaccaagctgtttcctaccaggtcacacactgttgca

SEQ ID NO: 41 - DNA, 100 bp, Homo Sapien
tgtgttcaatagatttaggagcagaaatgcaaggggctgcatgacctaccaggacagaactttccccaattacagggtgactcacagccgcattggtgac

SEQ ID NO: 42 - DNA, 100 bp, Homo Sapien
cgctgccttccatctgctcccacttcaatcctctgtctctcgaggaactaggctccaacacggggatccaggttttcaatcagattgtgaagtcgaggcc

SEQ ID NO: 43 - DNA, 100 bp, Homo Sapien
atggtggacaaccgtggcttcatggtgactcggtcctataccgtgggtgtcatgatgatgcaccggacaggcctctacaactactacgacgacgagaagg

SEQ ID NO: 44 - DNA, 100 bp, Homo Sapien; identical to SEQ ID NO: 27

SEQ ID NO: 45 - DNA, 100 bp, Homo Sapien
cagaaatcttgaaggcaggcgcaaacgggcataaattggagggaccactgggtgagagaggaataaggcggcccagagcgaggaaaggattttaccaaag

SEQ ID NO: 46 - DNA, 100 bp, Homo Sapien
tcctcctgttcgacagtcagccgcatcttcttttgcgtcgccagccgagccacatcgctcagacaccatggggaaggtgaaggtcggagtcaacggattt

SEQ ID NO: 47 - DNA, 100 bp, Homo Sapien
gcggcggaaaatagcctttgccatcactgccattaagggtgtgggccgaagatatgctcatgtggtgttgaggaaagcagacattgacctcaccaagagg

SEQ ID NO: 48 - DNA, 22 bp, Homo Sapien
cctgttcgacagtcagccgcat

SEQ ID NO: 49 - DNA, 22 bp, Homo Sapien
gactccgaccttcaccttcccc

SEQ ID NO: 50 - DNA, 22 bp, Homo Sapien
gcggcggaaaatagcctttgcc

SEQ ID NO: 51 - DNA, 24 bp, Homo Sapien
cctcttggtgaggtcaatgtctgc

SEQ ID NO: 52 - DNA, 24 bp, Homo Sapien
caaatgggcttgaagctgcttacg

SEQ ID NO: 53 - DNA, 25 bp, Homo Sapien
gtgtagcacattctgtccctgaaca

SEQ ID NO: 54 - DNA, 25 bp, Homo Sapien
aaggaccaaaaggtgacaagggtga

SEQ ID NO: 55 - DNA, 25 bp, Homo Sapien
gaacttgagcttgtcctggtactcc

SEQ ID NO: 56 - DNA, 23 bp, Homo Sapien
gtcatcccaccatcaccaaagcc

SEQ ID NO: 57 - DNA, 23 bp, Homo Sapien
atcgggaaggcacctgcagaatg

SEQ ID NO: 58 - DNA, 22 bp, Homo Sapien
ttgcaaatgggtgtgacgcggt

SEQ ID NO: 59 - DNA, 22 bp, Homo Sapien
aagcaccgcactttgctctgct

SEQ ID NO: 60 - DNA, 25 bp, Homo Sapien
acgaacactcaatccagtttgctga

SEQ ID NO: 61 - DNA, 23 bp, Homo Sapien
tggaatttatgcccgtttgcgcc

SEQ ID NO: 62 - DNA, 25 bp, Homo Sapien
tggcacaacaggaagctgttgaagg

SEQ ID NO: 63 - DNA, 25 bp, Homo Sapien
acacatatttggcatggttctggct

SEQ ID NO: 64 - DNA, 25 bp, Homo Sapien
tcatgctaccctgaagtcactcagt

SEQ ID NO: 65 - DNA, 20 bp, Homo Sapien
aggtcatcacacgtgcgggc

SEQ ID NO: 66 - DNA, 21 bp, Homo Sapien
caggaaccagcgaagaccgct

SEQ ID NO: 67 - DNA, 23 bp, Homo Sapien
tggcagatcctcgatgcgaatgc

SEQ ID NO: 68 - DNA, 25 bp, Homo Sapien
cggtcacttcacactctttgctccc

SEQ ID NO: 69 - DNA, 24 bp, Homo Sapien
cggaagccactttgtctcccatga

SEQ ID NO: 70 - DNA, 23 bp, Homo Sapien
accatgaactggcatctccccct

SEQ ID NO: 71 - DNA, 24 bp, Homo Sapien
tggagcctagttcctcgagagaca

SEQ ID NO: 72 - DNA, 22 bp, Homo Sapien
ccgtggcttcatggtgactcgg

SEQ ID NO: 73 - DNA, 24 bp, Homo Sapien
agtagttgtagaggcctgtccggt

SEQ ID NO: 74 - DNA, 22 bp, Homo Sapien
ctccaagcccatccaggggcaa

SEQ ID NO: 75 - DNA, 23 bp, Homo Sapien
cagagtgaccttcccagtgccaa

SEQ ID NO: 76 - DNA, 24 bp, Homo Sapien
tggctctttgccgaaatgctagag

SEQ ID NO: 77 - DNA, 24 bp, Homo Sapien
gggggctgagcatttggaatgttt

SEQ ID NO: 78 - DNA, 20 bp, Homo Sapien
aggagctgccaaagccctga

SEQ ID NO: 79 - DNA, 22 bp, Homo Sapien
acctgctccatccacaacaccg

SEQ ID NO: 80 - DNA, 20 bp, Homo Sapien
ttgttcagtggctcacttcg

SEQ ID NO: 81 - DNA, 20 bp, Homo Sapien
ttcaatgggaagaggtcctg

SEQ ID NO: 82 - DNA, 20 bp, Homo Sapien
atttctgaggagcctgcaac

SEQ ID NO: 83 - DNA, 20 bp, Homo Sapien
cacatacattcccctgcctt

SEQ ID NO: 84 - DNA, 20 bp, Homo Sapien
gagcctgtcaagaggcaaag

SEQ ID NO: 85 - DNA, 20 bp, Homo Sapien
ctggggatcttcgaatgcta